SEQUENCE LISTING (15 sequences)

SEQ ID NO: 1 - protein, 1273 aa, SARS-CoV-2
  misc_feature (1)..(1273): S protein (amino acid)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3819 nt, SARS-CoV-2
  misc_feature (1)..(3819): S protein (CDS)
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauaaaguggaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 3 - protein, 218 aa, SARS-CoV-2
  misc_feature (1)..(218): S protein RBD (amino acid)
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 4 - RNA, 654 nt, SARS-CoV-2
  misc_feature (1)..(654): S protein RBD (CDS)
auguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaa

SEQ ID NO: 5 - protein, 266 aa, SARS-CoV-2
  misc_feature (1)..(266): S protein RBD / Fibritin (amino acid)
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 6 - RNA, 798 nt, SARS-CoV-2
  misc_feature (1)..(798): S protein RBD / Fibritin (CDS)
auguuuguguuucuugugcugcugccucuugugucuucucaguguguggugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaggcucccccggcuccggcuccggaucugguuauauuccugaagcuccaagagaugggcaagcuuacguucguaaagauggcgaauggguauuacuuucuaccuuuuuaggccggucccuggaggugcuguuccagggccccggc

SEQ ID NO: 7 - protein, 1273 aa, SARS-CoV-2
  misc_feature (1)..(1273): S protein PP (amino acid)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - RNA, 3819 nt, SARS-CoV-2
  misc_feature (1)..(3819): S protein PP (CDS) (V08)
auguuuguguuucuugugcugcugccucuugugucuucucagugugugaauuugacaacaagaacacagcugccaccagcuuauacaaauucuuuuaccagaggaguguauuauccugauaaaguguuuagaucuucugugcugcacagcacacaggaccuguuucugccauuuuuuagcaaugugacaugguuucaugcaauucaugugucuggaacaaauggaacaaaaagauuugauaauccugugcugccuuuuaaugauggaguguauuuugcuucaacagaaaagucaaauauuauuagaggauggauuuuuggaacaacacuggauucuaaaacacagucucugcugauugugaauaaugcaacaaauguggugauuaaagugugugaauuucaguuuuguaaugauccuuuucugggaguguauuaucacaaaaauaauaaaucuuggauggaaucugaauuuagaguguauuccucugcaaauaauuguacauuugaauaugugucucagccuuuucugauggaucuggaaggaaaacagggcaauuuuaaaaaucugagagaauuuguguuuaaaaauauugauggauauuuuaaaauuuauucuaaacacacaccaauuaauuuagugagagaucugccucagggauuuucugcucuggaaccucugguggaucugccaauuggcauuaauauuacaagauuucagacacugcuggcucugcacagaucuuaucugacaccuggagauucuucuucuggauggacagccggagcugcagcuuauuaugugggcuaucugcagccaagaacauuucugcugaaauauaaugaaaauggaacaauuacagaugcuguggauugugcucuggauccucugucugaaacaaaauguacauuaaaaucuuuuacaguggaaaaaggcauuuaucagacaucuaauuuuagagugcagccaacagaaucuauugugagauuuccaaauauuacaaaucuguguccauuuggagaaguguuuaaugcaacaagauuugcaucuguguaugcauggaauagaaaaagaauuucuaauuguguggcugauuauucugugcuguauaauagugcuucuuuuuccacauuuaaauguuauggagugucuccaacaaaauuaaaugauuuauguuuuacaaauguguaugcugauucuuuugugaucagaggugaugaagugagacagauugcccccggacagacaggaaaaauugcugauuacaauuacaaacugccugaugauuuuacaggaugugugauugcuuggaauucuaauaauuuagauucuaaagugggaggaaauuacaauuaucuguacagacuguuuagaaaaucaaaucugaaaccuuuugaaagagauauuucaacagaaauuuaucaggcuggaucaacaccuuguaauggaguggaaggauuuaauuguuauuuuccauuacagagcuauggauuucagccaaccaauggugugggauaucagccauauagagugguggugcugucuuuugaacugcugcaugcaccugcaacaguguguggaccuaaaaaaucuacaaauuuagugaaaaauaaaugugugaauuuuaauuuuaauggauuaacaggaacaggagugcugacagaaucuaauaaaaaauuucugccuuuucagcaguuuggcagagauauugcagauaccacagaugcagugagagauccucagacauuagaaauucuggauauuacaccuuguucuuuugggggugugucugugauuacaccuggaacaaauacaucuaaucagguggcugugcuguaucaggaugugaauuguacagaagugccaguggcaauucaugcagaucagcugacaccaacauggagaguguauucuacaggaucuaauguguuucagacaagagcaggaugucugauuggagcagaacaugugaauaauucuuaugaaugugauauuccaauuggagcaggcauuugugcaucuuaucagacacagacaaauuccccaaggagagcaagaucuguggcaucucagucuauuauugcauacaccaugucucugggagcagaaaauucuguggcauauucuaauaauucuauugcuauuccaacaaauuuuaccauuucugugacaacagaaauuuuaccugugucuaugacaaaaacaucuguggauuguaccauguacauuuguggagauucuacagaauguucuaaucugcugcugcaguauggaucuuuuuguacacagcugaauagagcuuuaacaggaauugcuguggaacaggauaaaaauacacaggaaguguuugcucaggugaaacagauuuacaaaacaccaccaauuaaagauuuuggaggauuuaauuuuagccagauucugccugauccuucuaaaccuucuaaaagaucuuuuauugaagaucugcuguuuaauaaagugacacuggcagaugcaggauuuauuaaacaguauggagauugccugggugauauugcugcaagagaucugauuugugcucagaaauuuaauggacugacagugcugccuccucugcugacagaugaaaugauugcucaguacacaucugcuuuacuggcuggaacaauuacaagcggauggacauuuggagcuggagcugcucugcagauuccuuuugcaaugcagauggcuuacagauuuaauggaauuggagugacacagaauguguuauaugaaaaucagaaacugauugcaaaucaguuuaauucugcaauuggcaaaauucaggauucucugucuucuacagcuucugcucugggaaaacugcaggauguggugaaucagaaugcacaggcacugaauacucuggugaaacagcugucuagcaauuuuggggcaauuucuucugugcugaaugauauucugucuagacuggauccuccugaagcugaagugcagauugauagacugaucacaggaagacugcagucucugcagacuuaugugacacagcagcugauuagagcugcugaaauuagagcuucugcuaaucuggcugcuacaaaaaugucugaaugugugcugggacagucaaaaagaguggauuuuuguggaaaaggauaucaucugaugucuuuuccacagucugcuccacauggagugguguuuuuacaugugacauaugugccagcacaggaaaagaauuuuaccacagcaccagcaauuugucaugauggaaaagcacauuuuccaagagaaggaguguuugugucuaauggaacacauugguuugugacacagagaaauuuuuaugaaccucagauuauuacaacagauaauacauuugugucaggaaauugugauguggugauuggaauugugaauaauacaguguaugauccacugcagccagaacuggauucuuuuaaagaagaacuggauaaauauuuuaaaaaucacacaucuccugauguggauuuaggagauauuucuggaaucaaugcaucuguggugaauauucagaaagaaauugauagacugaaugaaguggccaaaaaucugaaugaaucucugauugaucugcaggaacuuggaaaauaugaacaguacauuaaauggccuugguacauuuggcuuggauuuauugcaggauuaauugcaauugugauggugacaauuauguuauguuguaugacaucauguuguucuuguuuaaaaggauguuguucuuguggaagcuguuguaaauuugaugaagaugauucugaaccuguguuaaaaggagugaaauugcauuacaca

SEQ ID NO: 9 - RNA, 3819 nt, SARS-CoV-2
  misc_feature (1)..(3819): S protein PP (CDS) (V09)
auguucguguuccuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguuuaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcucuggauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcaccggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggaaucugcgccagcuaccagacacagacaaacagcccucggagagccagaagcguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcaggcgccgcucugcagauccccuuugcuaugcagauggccuaccgguucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauaugugcccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacaca

SEQ ID NO: 10 - protein, 41 aa, SARS-CoV-2
  misc_feature (1)..(41): Foldon (amino acid)
GSGYIPEAPRDGQAYVRKDGEWVLLSTFLGRSLEVLFQGPG

SEQ ID NO: 11 - RNA, 123 nt, SARS-CoV-2
  misc_feature (1)..(123): Foldon (CDS)
ggaucugguuauauuccugaagcuccaagagaugggcaagcuuacguucguaaagauggcgaauggguauuacuuucuaccuuuuuaggccggucccuggaggugcuguuccagggccccggc

SEQ ID NO: 12 - RNA, 47 nt, Artificial Sequence
  Synthetic 5′-UTR (hAg-Kozak)
aacuaguauucuucugguccccacagacucagagagaacccgccacc

SEQ ID NO: 13 - RNA, 278 nt, Artificial Sequence
  Synthetic 3′-UTR (FI element)
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 14 - RNA, 110 nt, Artificial Sequence
  Synthetic A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 15 - protein, 1273 aa, SARS-CoV-2; identical to SEQ ID NO: 1
  misc_feature (1)..(1273): Spike protein from the Wuhan strain of SARS-CoV-2

(19) United States
(12) Patent Application Publication
(10) Pub. No.: US 2024/0339174 A1
(43) Pub. Date: Oct. 10, 2024

(54) TECHNOLOGIES FOR EARLY DETECTION OF VARIANTS OF INTEREST

(71) Applicants: BioNTech SE, Mainz (DE); InstaDeep Ltd., London (GB)

(72) Inventors: Alexander Muik, Seeheim-Jugenheim (DE); Asaf Poran, Stoneham, MA (US); Yunpeng Liu, Cambridge, MA (US); Ugur Sahin, Mainz (DE); Karim Beguir, London (GB); Marcin Skwark, London (GB); Thomas Pierrot, Paris (FR); Yunguan Fu, London (GB)

(21) Appl. No.: 18/289,425

(22) PCT Filed: May 4, 2022
(86) PCT No.: PCT/US22/27730

§ 371 Date: Nov. 3, 2023

Related U.S. Application Data
(60) Provisional application No. 63/293,611, filed on Dec. 23, 2021.
(60) Provisional application No. 63/293,649, filed on Dec. 23, 2021.
(60) Provisional application No. 63/283,430, filed on Nov. 27, 2021.
(60) Provisional application No. 63/283,206, filed on Nov. 24, 2021.

(30) Foreign Application Priority Data
May 4, 2021 (GB) 2106376.3
May 7, 2021 (GB) 2106580.0

(51) Int. Cl. G16B 20/20; C07K 14/005; G16B 15/30; G16B 40/20

(52) U.S. Cl. CPC G16B 20/20; C07K 14/005; G16B 15/30; G16B 40/20; C12N 2770/20022

(57) ABSTRACT

The present disclosure provides technologies for identifying, characterizing, and/or monitoring sequences of a variant of a reference infectious agent (e.g., but not limited to viral variants, for example in some embodiments SARS-CoV-2 variants) for transmissibility factors and/or immune escape potential, and/or for detecting and/or monitoring variants in environmental or biological samples, and/or for designing, preparing, and/or administering vaccines for such variants.

### CROSS REFERENCE TO RELATED APPLICATIONS

This application claims the benefit of United Kingdom Patent Application No. GB 2106376.3 filed May 4, 2021, United Kingdom Patent Application No. GB 2106580.0 filed May 7, 2021, U.S. Provisional Application No. 63/283,206 filed Nov. 24, 2021, U.S. Provisional Application No. 63/283,430 filed Nov. 27, 2021, and U.S. Provisional Application No. 63/293,611 filed Dec. 23, 2021, and U.S. Provisional Application No. 63/293,649 filed Dec. 23, 2021, the contents of each of which are hereby incorporated herein in their entirety.

### BACKGROUND

The ongoing COVID-19 pandemic is leading to the discovery of hundreds of novel SARS-CoV-2 variants on a near daily basis. While most variants do not impact the course of the pandemic, some variants pose significantly increased risk when the acquired mutations allow better evasion of antibody neutralization in previously infected or vaccinated subjects, or increased transmissibility.

### SUMMARY

Viral mutations that allow an infection to escape from recognition by neutralizing antibodies are a concern in the development of effective therapies for infections, for example, SARS-CoV-2 infections. As new sequences continue to naturally emerge, the potential for generation of variants that are both highly transmissible and highly immune resistant creates a significant challenge for prevention and/or treatment of such infections. Experimental techniques that perform causal escape profiling of all single-residues in a viral protein generally require substantial effort to profile even a single viral strain, and testing the escape potential of many combinatorial mutations in many viral strains remains infeasible. While transmissibility and immune escape potential of a given variant can be assessed experimentally, such methods are typically resource intensive and time consuming and cannot be scaled to properly address the multitude of emergent variants.

The present disclosure, among other things, provides technologies for identifying, characterizing, and/or monitoring sequences of a variant of a reference infectious agent (e.g., but not limited to viral variants, for example in some embodiments SARS-CoV-2 variants) for transmissibility factors and/or immune escape potential, and/or for detecting and/or monitoring variants in environmental or biological samples, and/or for designing, preparing, and/or administering vaccines for such variants.

Variants differ from reference agents (e.g., reference infectious agents or reference vaccine agents) by amino acid sequence alteration(s) (e.g., one or more substitutions, additions, deletions, and/or inversions of a single amino acid or of a set of adjacent amino acids).

In some embodiments, provided technologies are relevant to variants that arise and/or spread in a particular geographic location or within a particular community of contacts. In some embodiments, provided technologies are relevant to variants with greater infectivity and/or morbidity than a relevant reference variant. In some embodiments, provided technologies are relevant to so-called “escape” variants, able to evade an immune response to a reference agent. In some such embodiments, such immune response occurs or has occurred as a result of infection with a reference agent; in some such embodiments, such immune response occurs or has occurred as a result of immunization with a reference agent. For example, in some embodiments, such a variant can be an escape variant that is able to evade immunity that subjects acquire through vaccines and/or prior infections.

In some embodiments, technologies described herein are useful for identifying variants (e.g., at a given time point or over a given period of time) that are considered as “High Risk Variants” (HRVs). HRV refers to variants are known or predicted to be potentially dangerous, for example, because they are of higher fitness (e.g., higher infectivity), higher immune evasion, or both.

In some embodiments, technologies described herein are useful for identifying variants (e.g., at a given time point or over a given period of time) that are considered as “Variants under Monitoring” (VUM). In some embodiments, a VUM relates to the WHO designation, for example, which refers to a variant with genetic changes that are suspected to affect virus characteristics with some indication that it may pose a future risk, but evidence of phenotypic or epidemiological impact is currently unclear, requiring enhanced monitoring and repeat assessment pending new evidence.

In some embodiments, technologies described herein are useful for identifying variants (e.g., at a given time point or over a given period of time) that are considered as “Variants of Interest” (VOI). In some embodiments, a VOI relates to the WHO designation, for example which refers to a variant: (1) with genetic changes that are predicted or known to affect virus characteristics such as transmissibility, disease severity, immune escape, diagnostic or therapeutic escape; and (2) identified to cause significant community transmission or multiple infectious disease (e.g., COVID-19) clusters, in multiple countries with increasing relative prevalence alongside increasing number of cases over time, or other apparent epidemiological impacts to suggest an emerging risk to global public health.

In some embodiments, technologies described herein are useful for identifying variants (e.g., at a given time point or over a given period of time) that are considered as “Variants of Concern” (VOC). In some embodiments, a VOC relates to the WHO designation, for example, which refers to a variant that meets the definition of a VOI (as described herein) and, through a comparative assessment, has been demonstrated to be associated with one or more of the following changes at a degree of global public health significance: (1) Increase in transmissibility or detrimental change in infectious disease (e.g., COVID-19) epidemiology; or (2) Increase in virulence or change in clinical disease presentation; or (3) Decrease in effectiveness of public health and social measures or available diagnostics, vaccines, therapeutics.

In particular embodiments, the present disclosure provides results of an in silico approach combining (1) modeling of one or more structural feature(s) of a viral protein that may be involved in a process of virus invasion of a host, and (ii) one or more protein transformer language models on such viral protein sequences to reliably rank variants (e.g., in some embodiments currently circulating variants and/or previously circulating variants) for transmissibility factors and/or immune escape potential. In some embodiments, modeling of one or more structural feature(s) of a viral protein comprises (i) determining impact of amino acid sequence alteration(s) on viral fitness (e.g., efficacy of viral cell entry, and/or its structure and/or function), which is indicative of infectivity or transmissibility potential; and (ii) determining likelihood of a mutated epitope to evade neutralization by an immune system, which is indicative of immune escape potential.

The present disclosure, among other things, recognizes the source of problems that are associated with the “grammaticality” approach (e.g., as described in Hie et al., Science 371 (2021)284-288) and provides a different approach that provide certain particular advantages, including for example by using a “log-likelihood” approach. The higher the log-likelihood of a variant, the more probable is the variant to occur from a language model perspective. The present disclosure, among other things, appreciates that the log-likelihood metric supports substitutions, insertions and deletions without requiring a reference. In some embodiments, modeling with one or more protein transformer languages comprises, based on machine learning, determination of a semantic change score, which indicates predicted variation in one or more biological functions between a variant and a reference viral polypeptide; and/or determination of log-likelihood, which is a measure to characterize a variant polypeptide.

The present disclosure, among other things, provides an insight that growth of certain variants can change over time and/or geographical locations and thus in some embodiments it is desirable to include such metric to determine infectivity potential of a given variant. The present disclosure, among other things, also appreciates that because there are changes over time, a single variant as determined by methods described herein does not necessarily have a single immune escape or infectivity score.

Among other things, in some embodiments, the present disclosure provides an insight that transmissibility and immune escape metrics can be combined for an automated Early Warning System (EWS) that is capable of evaluating new variants in such short period of time that enables risk monitoring of variant lineages in near real time. In some embodiments, such an EWS can be trained on large datasets of sequence data (e.g., comprising genomic sequences and/or protein sequences) of known infectious agents (e.g., viral agents of interest, for example in some embodiments SARS-CoV-2, as well as known variants thereof) in an unsupervised manner and can predict variants that may arise, or may be prevalent or rapidly spreading in a certain region. In some particular embodiments, the present disclosure provides EWS technologies for detection and/or characterization of viral variants, and specifically SARS-CoV-2 variants. In some embodiments, such technologies can be useful for predicting which SARS-CoV-2 variants are likely to be variants of interest.

In some embodiments, provided technologies may be or include one or more immunogenic compositions (e.g., vaccines) that deliver a variant sequence comprising one or more amino acid substitutions identified using technologies described herein and/or methods (e.g., of making, using, assessing, etc.) such immunogenic compositions. In some embodiments, variants of interest may be potential escape variants (e.g., variants with an increased likelihood of being able to evade a subject's immune response). In some embodiments, provided technologies can be useful for designing and/or manufacturing immunogenic compositions (e.g., vaccines) directed to a variant of a reference infectious agent (e.g., but not limited to viral variants, for example, in some embodiments, SARS-CoV-2 variants). In some embodiments, provided technologies may be useful for prevention and/or treatment of an infection associated with a viral protein of interest.

In one aspect, provided herein is a method for assessing risk for a variant polypeptide. Such a method comprises: (A) providing an amino acid sequence of the variant polypeptide, which comprises one or more amino acid modifications relative to one or more reference viral polypeptides; (B) modeling one or more structural features of the variant polypeptide that are involved in viral invasion of a host; (C) determining, based on sequence data associated with the viral polypeptide, distance of each of the one or more amino acid modifications relative to the corresponding amino acids in the one or more the reference viral polypeptide; and (D) designating the variant polypeptide as a variant with elevated risk when the variant polypeptide is characterized in that: (a) it has an immune escape score that (i) satisfies a pre-determined immune escape threshold indicating likelihood of the variant polypeptide to be detected and neutralized by antibodies; and/or (ii) is ranked higher than at least one or more other variant polypeptides and/or reference viral polypeptides; and (b) it has an infectivity score that (i) satisfies a pre-determined infectivity threshold indicating level of viral fitness; and/or (ii) is ranked higher than at least one or more other variant polypeptides and/or reference viral polypeptides.

Another aspect provided herein is a method for assessing risk for a plurality of variant polypeptides. In some embodiments, such a plurality of variant polypeptides may comprise one or more currently circulating variants and/or one or more previously circulating variants. In some embodiments, such a method comprises: (A) providing a plurality of amino acid sequences of the variant polypeptides, wherein each of the variant polypeptides comprises one or more amino acid modifications relative to one or more reference viral polypeptides; (B) ascertaining, for each of the variant polypeptides, an immune escape score (indicative of likelihood of its detection and neutralization by antibodies) and an infectivity score (indicative of likelihood of its viral fitness) by performing the following processes: (a) modeling one or more structural features of each variant polypeptide that are involved in viral invasion of a host; and (b) determining, based on sequence data associated with the viral polypeptide, distance of each of the one or more amino acid modifications relative to the corresponding amino acids in the one or more reference viral polypeptides; (C) ranking risk of the variant polypeptides in the plurality by referencing respective combined scores of the immune escape score and the infectivity score; and (D) designating a variant polypeptide as a variant polypeptide with elevated risk when its combined score is ranked higher than that of at least one other variant polypeptide in the plurality.

In some embodiments, ranked variant polypeptides can be characterized in that (a) they each have an immune escape score that satisfies a pre-determined immune escape threshold indicating likelihood of the variant polypeptide to be detected and neutralized by antibodies; and (b) they each have an infectivity score that satisfies a pre-determined infectivity threshold indicating level of viral fitness (e.g., efficacy of viral cell entry, and/or its structure and/or function).

In some embodiments, all variant polypeptides of a plurality to be assessed in methods described herein share an overall amino acid sequence identity of at least 80% (including, e.g., at least 85%, at least 90%, at least 95%, at least 96%, at least 97%, at least 98%, or higher) with each other. In some embodiments, all variant polypeptides of a plurality to be assessed in methods described herein share an overall amino acid sequence identity of at least 80% (including, e.g., at least 85%, at least 90%, at least 95%, at least 96%, at least 97%, at least 98%, or higher) with one or more reference viral polypeptides. In some embodiments, one or more reference viral polypeptides may comprise a wild-type parental strain. In some embodiments, one or more reference viral polypeptides may comprise a known variant (e.g., in some embodiments a dominant variant spreading in certain geographical locations and/or spreading among global populations.).

In some embodiments, technologies provided herein are particularly amenable to SARS-CoV-2 (e.g., SARS-CoV-2 Spike polypeptide) variants. In some embodiments, a SARS-CoV-2 variant may be a naturally occurring variant. In some embodiments, a SARS-CoV-2 variant may be a designed or engineered SARS-CoV-2 variant. In some embodiments, technologies provided herein are particularly useful for assessing risk of variants having one or more amino acid modifications present in Receptor Binding Domain (RBD) and/or N-terminal domain of the Spike polypeptide.

In some embodiments, methods for assessing risk of one or more variants comprises calculation of an immune escape score. In some embodiments, calculation of an immune escape score comprises calculation of an epitope alteration score, which in some embodiments may be determined by identifying one or more sequence alterations in a viral polypeptide (e.g., SARS-CoV-2 Spike polypeptide), and comparing the location and/or nature of the one or more sequence alterations to amino acid loci associated with disrupting binding interactions between neutralizing antibodies and a viral polypeptide (e.g., SARS-CoV-2 Spike polypeptide).

In some embodiments, an immune escape score is calculated using a machine learning language model. For example, in some embodiments, calculation of an immune escape score comprises determining a semantic change score for a variant polypeptide relative to one or more reference viral polypeptides (e.g., as described herein). In some embodiments where an immune escape score is computed for a SARS-CoV-2 variant, one or more reference viral polypeptides is or comprises a Wuhan SARS-CoV-2 spike polypeptide or portion thereof, and/or a natural or engineered SARS-CoV-2 Spike polypeptide or potion thereof (e.g., a D614G SARS-CoV-2 spike polypeptide or portion thereof).

In some embodiments, a machine learning language model utilized in methods described herein has been trained on a database comprising relevant viral sequences (e.g., SARS-CoV-2 polypeptide sequences). In some embodiments, such a database may comprise genomic sequences and/or polypeptide sequences of relevant viral sequences (e.g., SARS-CoV-2 polypeptide sequences). In some embodiments, such a database is or comprises a GISAID database.

In some embodiments, immune escape score is calculated using a combination of a semantic change score and an epitope alteration score. In some embodiments, an average of a semantic change score and an epitope alteration score is used to calculate an immune escape score.

In some embodiments, methods described herein may comprise characterizing computational assessment of a variant by referencing in vitro pseudovirus neutralization test results. For example, in some embodiments, an immune escape score, a semantic change score, and/or an epitope alteration score correlates with an in vitro pseudovirus neutralization test result. In some embodiments, such a correlation may be based on a least squares regression line. In some embodiments, a variant polypeptide designated as a variant with elevated risk is characterized in that when assessed with a pseudovirus neutralization assay, the variant polypeptide exhibits a reduction in observed 50% pseudovirus neutralization titer (pVNT50) by at least 10% (including, e.g., at least 20%, at least 30%, at least 40%, at least 50%, at least 60%, at least 70%, at least 80%, at least 90% or higher) as compared to one or more reference viral polypeptides (e.g., ones described herein). In some embodiments, such one or more reference viral polypeptides is or comprises a wild-type SARS-CoV-2 (Wuhan strain) pseudotyped VSV.

In some embodiments, methods for assessing risk of one or more variants comprises calculation of an infectivity score. In some embodiments, calculation of an infectivity score comprises calculation of a viral polypeptide receptor (e.g., ACE2 receptor in the context of a SARS-CoV-2) binding score, which is a measure of binding affinity between a viral polypeptide receptor (e.g., ACE2 receptor) and a viral polypeptide (e.g., a Spike polypeptide). In some embodiments, a binding affinity between a viral polypeptide receptor (e.g., ACE2 receptor) and a viral polypeptide (e.g., Spike polypeptide) is an in silico binding affinity. For example, in some embodiments, such an in silico binding affinity is a predetermined value that was determined in silico using structural modeling. In some embodiments, interaction between a variant polypeptide and a viral polypeptide (e.g., ACE2 polypeptide) is modeled and/or calculated through in silico docking experiments. In some embodiments, an in silico binding affinity is a predetermined value that was determined in silico by calculating the median difference in solvent accessible surface between bound and unbound states of a viral polypeptide (e.g., Spike polypeptide such as in some embodiments, receptor binding domain (RBD) of a Spike polypeptide).

In some embodiments, calculation of an infectivity score comprises determination of similarity of the variant polypeptide to other known variants (e.g., variants that have been known to grow rapidly), for example, by determination of a log-likelihood score, which indicates the likelihood of occurrence of a given input sequence. In some embodiments, a log-likelihood is computed from probabilities over amino acid residues returned by a language model. In some embodiments, a log-likelihood is calculated as the sum of log-probabilities over all the positions of the spike protein amino-acids.

In some embodiments, calculation of an infectivity score further comprises determining growth rate of a variant polypeptide and/or referencing growth rate of a viral polypeptide having an amino acid sequence that is at least 80% (e.g., at least 85%, at least 90%, at least 95%, at least 96%, at least 97%, at least 98%, at least 99%, or higher) identity to the sequence of the variant polypeptide. In some embodiments, growth rate of a variant polypeptide can change (e.g., declining or increasing) over time.

Technologies described herein are useful for identifying and/or monitoring emergence of a variant of concern. For example, variants of concern can be identified using methods described herein. In some embodiments, when a variant of concern is identified, methods for tracking and/or containment of the variant of concern can be implemented. For example, in some embodiments, environmental monitoring of an identified variant of concern may be implemented, for example, in designated spaces, such as, e.g., in public spaces (e.g., schools, child care setting, mass transportation, hospitals, etc.), and/or in wastewater or sewages. In some embodiments, contact tracing of an identified variant of concern may be implemented. In some embodiments, when a variant of concern is identified using methods described herein, a vaccine against the variant of concern can be manufactured.

Methods of producing a vaccine against a viral variant (e.g., a SARS-CoV-2 variant) are also provided herein. For example, such a method comprises identifying at least one variant polypeptide of interest using any one of methods described herein, and producing, within a period of time from the identification of at least one variant polypeptide of interest, a vaccine comprising a polypeptide or comprising a nucleic acid encoding the polypeptide, wherein the polypeptide comprises at least one variant polypeptide of interest or immunogenic fragment thereof. In some embodiments, a vaccine (e.g., as described herein) is produced with a period of time that is no more than 12 weeks (including, e.g., no more than 11 weeks, no more than 10 weeks, no more than 9 weeks, no more than 8 weeks, no more than 7 weeks, no more than 6 weeks, no more than 5 weeks, no more than 4 weeks, no more than 3 weeks, no more than 2 weeks, or shorter) from the identification of at least one variant polypeptide of interest.

In some embodiments, a method of producing a vaccine against a viral variant (e.g., a SARS-CoV-2) can comprise identifying a plurality of variant polypeptides of interest using any one of methods described herein. In some embodiments, a vaccine may comprise one or more polypeptides or one or more nucleic acids encoding the one or more polypeptides, wherein the polypeptide(s) comprise(s) one or more variant polypeptides of interest or immunogenic fragments thereof. In some embodiments, a vaccine comprises a polyepitopic polypeptide comprising the identified plurality of variant polypeptides of interest or immunogenic fragment(s) thereof or a nucleic acid encoding the polyepitopic polypeptide. In some embodiments, a vaccine comprises two or more polypeptides, or two or more variants of the same polypeptide (e.g., two or more variants of a SARS-CoV-2 Spike polypeptide), or an immunogenic fragment of any of the foregoing, or a nucleic acid comprising a sequence encoding any of the same, wherein at least one of such polypeptides (or fragments thereof) has been identified as a variant of interest as described herein.

A further aspect of the present disclosure provides a viral polypeptide (e.g., SARS-CoV-2 Spike polypeptide) or an immunogenic fragment or variant thereof, or a nucleic acid comprising a sequence encoding the same, wherein the viral polypeptide (e.g., Spike polypeptide) or the immunogenic fragment or variant is determined as a variant of concern by performing or utilizing technologies described herein. Provided herein is also a viral polypeptide (e.g., SARS-CoV-2 Spike polyepitopic polypeptide), or a nucleic acid comprising a sequence encoding the same, wherein the viral polyepitopic polypeptide (e.g., Spike polyepitopic polypeptide) comprises at least two variant polypeptides as determined to be variants of interest by performing or utilizing technologies described herein. In some embodiments, a nucleic acid is or comprises RNA (e.g., in some embodiments mRNA). In some embodiments, a nucleic acid is or comprises DNA. Such polypeptides and/or nucleic acids can be useful for producing vaccine compositions. Accordingly, in certain aspects, a vaccine composition comprising such a polypeptide or nucleic acid is also described herein.

In one aspect, technologies described herein can be useful for vaccination. For example, in some embodiments, a method of vaccination comprises administering to a subject or a population of subjects a vaccine that is manufactured to fight against a variant as determined to be a high risk variant using methods described herein. In some embodiments, such a subject or a population of subjects has previously been exposed to a reference viral polypeptide (e.g., SARS-CoV-2 polypeptide). For example, in some embodiments, such a subject or a population of subjects has previously been vaccinated against a reference viral polypeptide (e.g., SARS-CoV-2 polypeptide), while in some embodiments, such a subject or a population of subjects has previously been infected with a reference viral polypeptide (e.g., SARS-CoV-2 polypeptide). In some embodiments, such a subject or a population of subjects has not been previously infected with a reference viral polypeptide (e.g., a SARS-CoV-2 polypeptide). In some embodiments, such a subject or a population of subjects has no known exposure to an identified variant polypeptide(s) in the vaccine.

As described herein, provided technologies permit assessment of whether a particular polypeptide (e.g., a particular variant of a SARS-CoV-2 spike protein, or fragment thereof) is likely to escape an immune response to a related polypeptide (e.g., a reference SARS-CoV-2 spike protein, or fragment thereof). In some embodiments, a provided method of vaccination with a vaccine that is manufactured to fight against a high risk variant using methods described herein comprises vaccination with a vaccine determined to induce an immune response that the relevant high risk variant is unlikely to escape. In some embodiments, the present disclosure provides technologies for vaccinating a subject or population of subjects exposed (or at risk of exposure) to a high risk variant (e.g., determined as described herein) with a viral polypeptide (e.g., SARS-CoV-2 Spike polypeptide) or an immunogenic fragment or variant thereof, or a nucleic acid comprising a sequence encoding the same, that induces or enhances an immune response that the relevant high risk variant has been determined (e.g., as described herein) to be unlikely to escape.

In some embodiments, provided technologies include vaccination of a subject or population with a viral polypeptide (e.g., SARS-CoV-2 Spike polypeptide) or an immunogenic fragment or variant thereof, or a nucleic acid comprising a sequence encoding the same, that induces or enhances an immune response that a plurality of variants of the polypeptide (e.g., two or more such variants) are unlikely to escape. In some such embodiments, different variants may have been detected in a common geographic location; in some embodiments, different variants may have been detected in different geographic locations. Alternatively or additionally, in either case, in some embodiments, different variants may have been detected within a common time window; in some embodiments, different variants may have been detected at different points in time.

In some embodiments, provided technologies comprise vaccinating subjects in different locations and/or at different times, with the same vaccine composition. In some embodiments, provided technologies comprise vaccinating subjects in different locations and/or at different times with different vaccine compositions (e.g., as may reflect circulating strains at the relevant location(s) and/or times, e.g., where vaccines are selected for administration when locally and/or temporally relevant strains are determined to have a low probability of escaping immune responses induced or enhanced by the vaccine(s) selected for administration.

An Early Warning System (EWS) for detecting one or more variants of interest is also provided herein. Such a system comprises technologies for identifying a viral variant of interest (e.g., a SARS-CoV-2 variant of interest) using technologies described herein. In some embodiments, a viral variant of interest (e.g., a SARS-CoV-2 variant of interest) can be identified within a period of time that is less than 6 weeks (including, e.g., less than 5 weeks, less than 4 weeks, less than 3 weeks, less than 2 weeks, less than 1 week, less than 6 days, less than 5 days, less than 4 days, less than 3 days, less than 2 days, less than 1 day, or shorter).

In some embodiments, an EWS further comprises technologies (e.g., automated technologies) for notifying relevant health agencies (e.g., local, regional and/or other health agencies), monitoring agencies, and/or communities (e.g., in some embodiments those related by employment) of an identified variant of interest. In some embodiments, such a notification can be performed within 8 weeks (including, e.g., within 7 weeks, within 6 weeks, within 5 weeks, within 4 weeks, within 3 weeks, within 2 weeks, within 1 week, or shorter) from the identification of a variant of interest using technologies described herein.

In some embodiments, an EWS further comprises technologies (e.g., automated technologies) for contact tracing of an identified variant of interest. In some embodiments, an EWS further comprises technologies (e.g., automated technologies) for periodic sampling and/or environmental monitoring of an identified variant of interest. In some embodiments, an EWS further comprises technologies (e.g., automated technologies) for reporting the identified variant of interest.

### BRIEF DESCRIPTION OF THE DRAWINGS

FIG. 1. A schematic of the Early Warning System (EWS): structural modeling methods and natural language processing techniques to enable risk level estimation of SARS-CoV-2 variants in real time. (A) Structural modeling is used to predict the binding affinity of SARS-CoV-2 spike protein to host ACE2, and to score the mutated epitope regarding its impact on immune escape. (B) Language modeling (e.g., performed via machine learning modeling) is used to extract implicit information from unlabeled data for the hundreds of thousands of registered variants in the GISAID database. (C) EWS relies on the information from A and B to compute an immune escape score and an infectivity score (also known as “a fitness prior score”), which, taken together, present a more comprehensive view of the SARS-CoV-2 variant landscape. Both scores can be combined to obtain a single score, based on the notion of Pareto optimality and dubbed Pareto score, that represents a variant's risk. The higher the Pareto score, the fewer variants with higher immune escape and fitness prior scores. (D) Schematic of AI model structure for assessing semantic change and log-likelihood. Once trained (FIG. 7A), the model receives as input a variant Spike protein sequence, and returns an embedding vector of the spike protein sequence as well as probabilities over amino-acids for each residue. The embedding vector is used to calculate semantic change from the Wuhan and D614G variants while the probabilities are used to compute the log-likelihood.

FIG. 2. Surface of a SARS-CoV-2 spike protein in ‘one RBD up’ conformation (PDB id: 7kdl) (Gobeil et al., 2021), colored by the frequency of contact of surface residues with neutralizing antibodies (brighter, warmer color corresponds to more antibody binding), out of 768 unique epitopes combinations of 800 antibody orientations, present in 310 PDB files. (A) Comparison of antibody propensity between a wild-type variant and a Gamma (P.1) variant. Left column: side view. Right column: top view. Top row: antibody binding propensity of a wild type variant. Bottom row: antibody binding propensity of Gamma (P.1) variant. (B) Comparison of antibody propensity of a wild-type variant with a Beta (B.1351) variant or an Omicron (B.1.1.529) variant. Middle and bottom row depict the number of evaded epitopes in a Beta (B.1.351) and Omicron (B.1.1.529). Left column: side view. Right column: top view

FIG. 3. In silico predicted scores for immune escape and infectivity correlate with in vitro data. (A) Validation of the immune escape metric with pseudovirus neutralization test (pVNT) results. Relationships of the epitope score, semantic change score, and combined immune escape score with the observed 50% pseudovirus neutralization titer (pVNT₅₀) reduction are shown across n=17 selected SARS-CoV-2 variants of interest, along with a linear regression dash line (e.g., in some embodiments linear regression can be performed by the least squares regression method). Cross-neutralization of n=12-40 BNT162b2-immune sera was assessed against vesicular stomatitis virus (VSV)-SARS-CoV-2-S pseudoviruses. pVNT₅₀ reduction compared to wild-type SARS-CoV-2 (Wuhan strain) spike pseudotyped VSV is given in percent. Variants for which experimentally measured geometric mean pVTN50 increased compared to the Wuhan strain have been assigned a pVTN50 reduction of 0 (equal to wild type). Epitope score (based on structural modeling) indicates the number of known neutralizing antibodies (max. n=310) whose binding epitope is affected by the SARS-CoV-2 variants' mutations. Semantic change score (based on machine learning) indicates the predicted variation in the biological function between a variant and wild-type SARS-CoV-2. For the semantic change score the distance in embedding space between the sequence in question and a reference (WT+D614G spike protein) is compared. Sequences have then been ranked with respect to this distance and the resultant rank has been scaled in the range of [0, 100]. The immune escape score is calculated as the average of the scaled epitope score and the scaled semantic change score. (B) Validation of a first component of the infectivity (fitness prior) metric, capturing the ACE2 binding propensity. The ACE2 binding score is ranked and scaled analogously to infectivity (fitness prior) components, such that variants with largest interface size are assigned a score of 100, smallest—0. (C) Validation of a second component of the infectivity (fitness prior) metric, capturing the log-likelihood. Log-likelihood of all variants reporting the same submission count is averaged and closely recapitulates the number of submissions it is compared against.

FIG. 4. Combining immune escape and infectivity for continuous monitoring. (A) Snapshot of lineages in terms of Infectivity and Immune escape score on Jan. 17, 2021. Marker size indicates the number of submissions of each lineage. (B) Progression of the infectivity and immune escape scores of main lineages flagged by WHO through time from the early snapshot (January 2021) to the later snapshot (end of August 2021). Each dot represents the position of the center of mass of a given lineage on each month. (C) Snapshot on Sep. 1, 2021. (D-F) Progression of the infectivity or growth score and immune escape scores of main lineages designated by WHO through time from the early snapshot (January 2021) to the later snapshot (September 2021). Each dot represents the position of the center of mass of a given lineage on each month. (D) and (E) demonstrate the progression using infectivity score with and without growth respectively. (F) shows the progression using only growth. (G) Kernel density estimate (KDE) of non-designated and designated variants distributions on Jan. 17, 2021. (H) Kernel density estimate plot on Sep. 1, 2021.

FIG. 5. EWS flags High Risk Variants ahead of their WHO designation. (A) Cumulative sum of all cases of a given variant lineage (in log scale) over time. Vertical lines indicate the date of WHO designation of a given variant (green dot-dashed)) vs. date of flagging by the EWS (red dashed, using a weekly watch-list size of 20 variants). (B). Lead time of EWS detection ahead of WHO designation vs. minimum weekly watch-list size required. (C). Detection results (measured in days of lead time vs. WHO designation) from selecting 20 variants per week at random (repeated 100 times) compared with selecting top 20 variants by growth score (light-green cross) and immune escape score (green circle). Boxplots borders indicate 25th and 75th percentiles, horizontal lines indicate median, and whiskers indicate minimal and maximal values. If a variant cannot be detected with growth or immune escape score, the marker is not displayed.

FIG. 6. Maximum lead time of EWS detection ahead of WHO designation vs. required weekly watch-list size.

FIG. 7. Machine learning modeling. (A) A transformer language model is pre-trained on all the protein sequences registered in the Uniref dataset. Every week, the model is fine-tuned over all the spike protein sequences registered so far by the GISAID initiative. Both the pre-training and fine-tuning use the same protocol. Amino-acids of a protein sequence are randomly masked. The model predicts probabilities over amino-acids at each residue position, both for residues that were masked and not masked. A loss function evaluates the sum over the masked residues of the log-probability of the correct predictions. A gradient of this loss is computed and used to update the model's parameters so as to increase the loss function. (B) Once fine-tuned, the model is used to compute the semantic change and the log-likelihood to characterize a spike protein sequence. The output of the last transformer layer is averaged over the residues to obtain an embedding z of the protein sequence. The embedding of the Wuhan strain z_{wuhan} and the embedding of the D614G variant z_{D614G} are computed once for all. The semantic change is computed as the sum of the euclidean distance between the z and z_{wuhan} the euclidean distance between z and z_{D614G}. The log-likelihood is computed from the probabilities over the residues returned by the model. It is calculated as the sum of the log-probabilities over all the positions of the spike protein amino-acids.

FIG. 8. Semantic change vs epitope alteration score (EAS). The number of known nAbs whose binding epitope is affected by a distinct SARS-CoV-2 variants' mutations was defined as the epitope alteration score (EAS).

FIG. 9. Cross-neutralization of BNT162b2-immune sera against VSV-SARS-CoV-2-S pseudoviruses bearing the Spike protein of selected SARS-CoV-2 variants. Serum samples were obtained from participants in the BNT162b2 vaccine phase-III trial on day 28 or day 43 (7 or 21 days after Dose 2). A recombinant vesicular stomatitis virus (VSV)-based SARS-CoV-2 pseudovirus neutralization assay was used to measure neutralization. The pseudoviruses tested incorporated the ancestral SARS-CoV-2 Wuhan Hu-1 Spike or Spikes with substitutions present in B.1.1.7+E484K (Alpha), B.1.351 (Beta), P.1 (Gamma), B.1.617.2 (Delta), AY.1 (Delta), B.1.427/B.1.429 (Epsilon), B.1.526 (Iota), B.1.617 (Kappa), C.37 (Lambda), C.37*(Lambda), A.VOI.V2, B.1.517, B.1.258, B.1.160, and B.1.1.529 (Omicron) (Table 11). (A) Pseudovirus 50% neutralization titers (pVNT₅₀) are shown. Dots represent results from individual serum samples. Lines connect paired neutralization analyses performed within one experiment. In total 8 experiments were performed covering the listed SARS-CoV-2 variants always referencing variant-specific neutralization to the Wuhan reference. (B) pVNT₅₀ against B.1.1.529 (Omicron) are shown. Dots represent results from individual serum samples. Lines connect paired neutralisation analyses performed within one experiment. (C) Ratio of pVNT₅₀ between SARS-CoV-2 variant and Wuhan reference strain Spike-pseudotyped VSV. Dots represent results from individual serum samples. Horizontal bars represent geometric mean ratios, error bars represent 95% confidence intervals.

FIG. 10. Results of molecular simulations of RBD binding. The efficiency of Spike protein RBD binding to the ACE2 receptor was dictated by the combination of binding energy (A; the lower the better) and size of the interface (B). Both boxen plots depict distribution of these values across performed RBD binding simulations for circulating spike protein variants. Note, that while larger interfaces may be difficult to form, they are also more difficult to break. Strikingly, Omicron, despite its heavily mutated RBD has a relatively large interface and a binding affinity around the 25th percentile of the background distribution (‘Other’).

FIG. 11. Log-likelihood score corrects for large mutation count. (Top) Snapshot of lineages in terms of log-likelihood ranked without correction for large mutation count and immune escape score on Nov. 23, 2021. Marker size indicates the number of submissions of each lineage. (Bottom) Same plot where the log-likelihood ranked without correction has been replaced by its corrected version. Note, that both plots are nearly identical, as highly mutated sequences comprise less than 1% of the entire data set. Nearly no change is observed between the plots, with concerning lineages residing on the second Pareto front, except for the emergence of B.1.1.529 (Omicron) as a clear outlier, practically alone in the first Pareto front, due to its high immune escape and extraordinary log-likelihood, given its high number of mutations. Additionally, the conditional log-likelihood score is nearly co-linear with expected prevalence of sequence in population (see FIG. 20)

FIG. 12. (A) Validation of a component of the fitness prior metric, capturing the ACE2 binding propensity. The ACE2 binding score is ranked and scaled analogously to fitness prior components, such that variants with the lowest energy are assigned a score of 100, highest—0. (B) Validation of a second component of the fitness prior metric, capturing the log-likelihood. Sequences are grouped into bins based on their submission count and the log-likelihood scores and number of submissions were averaged per bin. The first ten bins correspond to count 1 to 10. The next 10 bins are equally split between counts 11 and 1000 such that each bin has a similar number of sequences. The last two bin contains all sequences having a submission count from 1000 to 10,000 and sequences having more than 10,000 submissions.

FIG. 13. Validation of the immune escape metric with pseudovirus neutralization test (pVNT) results. Relationships of the epitope alteration score, semantic change score, and combined immune escape score with the observed 50% pseudovirus neutralization titer (pVNT50) reduction are shown across n=19 selected SARS-CoV-2 variants of interest, including Omicron (B.1.1.529). Cross-neutralization of n=12-40 BNT162b2-immune sera was assessed against vesicular stomatitis virus (VSV)-SARS-CoV-2-S pseudoviruses. pVNT50 reduction compared to wild-type SARS-CoV-2 (Wuhan strain) Spike pseudotyped VSV is given in percent. Variants for which experimentally measured geometric mean pVNT50 increased compared to the Wuhan strain have been assigned a pVNT50 reduction of 0 (equal to wild type). Epitope alteration score (based on structural modeling) indicates the number of known neutralizing antibodies (max. n=310) whose binding epitope is affected by the SARS-CoV-2 variants' mutations. Semantic change score (based on machine learning) indicates the predicted variation in the biological function between a variant and wild-type SARS-CoV-2. For the semantic change score, the distance in embedding space between the sequence in question and a reference (WT+D614G Spike protein) is compared. Sequences have then been ranked with respect to this distance and the resultant rank has been scaled in the range of [0, 100]. The immune escape score is calculated as the average of the scaled epitope alteration score and the scaled semantic change score. Dashed lines represent the linear regression

FIG. 14. Combining immune escape and fitness prior for continuous monitoring. (A) Snapshot of lineages in terms of fitness prior and immune escape score on respectively from left to right Jan. 17, 2021, Sep. 1, 2021 and Nov. 23, 2021. Marker size indicates the number of submissions of each lineage. (B) Given a large number of lineages, densities were used instead of points clouds for visualization. Densities of non-designated and designated variants on Jan. 17, 2021, Sep. 1, 2021 and Nov. 23, 2021 are represented. The density contour plot is computed by grouping points specified by their coordinates into bins and calculating contours using counts. (C) Progression of the fitness prior and immune escape scores of main lineages designated by WHO through time from the early snapshot (January 2021) to the later snapshot (September 2021). Each dot represents the position of the center of mass of a given lineage on each month. The left and center plot demonstrates the progression using fitness prior score with and without growth respectively. The right plot shows the progression using only growth.

FIG. 15. Combining immune escape and fitness prior for continuous monitoring. (Left) Density contour plot of sequences on Jan. 17, 2021. Sequences are split into two groups: WHO designated ones and other non-designated ones. (Center) Density contour plot on Sep. 1, 2021. (Right) Density contour plot on Nov. 23, 2021.

FIG. 16. EWS flags High Risk Variants ahead of their WHO designation. (A) Cumulative sum of all cases of a given variant lineage (in log scale) over time. Vertical lines indicate the date of WHO designation of a given variant (green dot-dashed) vs. date of flagging by the EWS (red dashed, using a weekly watch-list size of 20 variants). (B). Lead time of EWS detection ahead of WHO designation vs. minimum weekly watch-list size required (in log scale). (C). Detection results (measured in days of lead time vs. WHO designation) from selecting 20 variants per week at random (repeated 100 times) compared with selecting top 20 variants by growth score (light-green cross) and immune escape score (green circle). Boxplots borders indicate 25th and 75th percentiles, horizontal lines indicate median, and whiskers indicate minimal and maximal values. If a variant could not be detected with growth or immune escape score, the marker was not displayed. (D) Variants detected when using Epitope Alteration Score, Semantic Score and Immune Escape Score components of the EWS. The left bar chart displays the number of variants detected by EWS using different scores. The right part visualizes whether a WHO designated variant was detected in advance using different scores, where green dots indicate early detections and grey dots mean the variants are not detected in advance.

FIG. 17. The maximum lead time of EWS detection ahead of WHO designation vs. required weekly watch-list size. With a weekly watch list of 200 sequences, all WHO designated variants are detected, including Delta.

FIG. 18. Metrics of anticipated reduction of the immune response. Semantic change and Epitope alteration score accurately segment the variant landscape, allowing to discriminate between variants that do not have immune escape propensity (B.1.429, WT), highly mutated, but neutralizable variants (P.1, B.1.160), and those with high potential for evading immune response (B.1.1.7, AY.1, B.1.351).

FIG. 19. Validation of a component of a fitness prior metric, capturing ACE2 binding propensity. Relationship of an ACE2 binding score with the experimentally determined ACE2 binding affinity (K_{D}, dissociation constant) are shown across n=19 RBD variants, along with a linear regression dash line. The ACE2 binding score is ranked and scaled analogously to fitness prior components, such that variants with the lowest energy are assigned a score of 100, highest—0.

FIG. 20. Validation of conditional log-likelihood scores. Sequences are grouped into bins based on their submission count and the conditional log-likelihood scores and number of submissions were averaged per bin. The first ten bins correspond to count 1 to 10. The next 10 bins are equally split between counts 11 and 1000 such that each bin has a similar number of sequences. The last two bin contains all sequences having a submission count from 1000 to 10,000 and sequences having more than 10,000 submissions. The data demonstrates that the mean conditional log-likelihood of sequences that are observed frequently in circulation is much higher than that of outlier, infrequent sequences.

### CERTAIN DEFINITIONS

About or Approximately: The term “about” or “approximately”, when used herein in reference to a value, refers to a value that is similar to the referenced value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Adult: As used herein, the term “adult” refers to a human eighteen years of age or older. In some embodiments, a human adult has a weight within the range of about 90 pounds to about 250 pounds.

Agent: In general, the term “agent”, as used herein, is used to refer to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amelioration: as used herein, refers to the prevention, reduction or palliation of a state, or improvement of the state of a subject. Amelioration includes, but does not require complete recovery or complete prevention of a disease, disorder or condition (e.g., radiation injury).

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies. etc. In some embodiments, relevant formats may be or include: Adnectins®; Affibodies®; Affilins®; Anticalins®; Avimers®; BiTE® s; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR® s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.])

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Antigen: The term “antigen”, as used herein, refers to an agent that elicits an immune response; and/or an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer), etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present disclosure are provided in a crude form. In some embodiments, an antigen is a recombinant antigen.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a N7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a DNA template, wherein, in addition to the GTP, an IVT system also contains a cap analog, e.g., as known in the art. Non-limiting examples of a cap analog include a m7GpppG cap analog or an N7-methyl-, 2′-O-methyl-GpppG ARCA cap analog or an N7-methyl-, 3′-O-methyl-GpppG ARCA cap analog, or any commercially available cap analogs, including, e.g., CleanCap (Trilink), EZ Cap, etc. In some embodiments, a cap analog is or comprises a trinucleotide cap analog.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Determine: In some embodiments, the methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Encapsulated: The term “encapsulated” is used herein to refer to substances that are completely surrounded by another material.

Epitope: as used herein, the term “epitope” refers to a moiety that is specifically recognized, or predicted to be recognized, by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Epitope Alteration Score: As used interchangeably herein, the terms “epitope alteration score” and “epitope score” both refer to a measure of alteration to a viral polypeptide at epitope positions. In some embodiments, such alteration can be characterized by the impact of mutation(s) in one or more epitopes of a viral variant on recognition by antibodies (e.g., neutralizing antibodies). For example, in some embodiments, such alteration can be characterized by determining the number of antibodies potentially escaped. In some embodiments, antibodies for characterization have been isolated from patients who have been vaccinated against a disease or who have previously been infected with a disease (e.g., SARS-CoV-2). In some embodiments, antibodies for characterization have previously been shown to bind a reference sequence. In some embodiments, an epitope alteration score can be determined by comparison of mutations in a variant candidate to one or more regions of a reference sequence that have previously been shown to bind antibodies (e.g., through structural data). In some embodiments, an epitope alteration score can be determined by enumerating the number of unique epitopes involving altered positions, as measured across one or more known antibody-viral polypeptide complex structures (e.g., all known antibody-viral polypeptide complex structures).

In some embodiments, an epitope alteration score is a measure of how many distinct epitopes are evaded by a variant candidate as compared to a reference sequence (e.g., as compared to a wild type sequence). In some embodiments, an epitope alteration score is computed based on known binding sites of antibodies, e.g., as reported in Protein Data Bank. In some embodiments, an epitope alteration score can change over time with identification of new epitope positions and/or discoveries of epitope-binding antibodies. In some embodiments, an epitope alteration score can be used to characterize degree of alteration of a SARS-CoV-2 Spike polypeptide at epitope positions, for example, in some embodiments by counting the number or percentage of antibodies potentially escaped. In various embodiments described herein, an epitope alteration score can be normalized such that it ranks between 0 and 100%.

Excipient: as used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to the generation of a gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Fed-batch process: The term “fed-batch process” as used herein refers to a process in which one or more components are introduced into a vessel, e.g., an in vitro transcription reaction, at some time subsequent to the beginning of a reaction. In some embodiments, one or more components are introduced by a fed-batch process to maintain its concentration low during a reaction. In some embodiments, one or more components are introduced by a fed-batch process to replenish what is depleted during a reaction.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Growth score: As used interchangeably herein, the term “growth,” “growth metric,” or “growth score” refers to a measure of the rate at which a given variant is growing in a subject population (e.g., at a given time). In some embodiments, a growth score refers to lineage-level growth. For example, in some embodiments, a growth score of a given variant can be determined by referencing growth of a parent species or a known variant of substantially the same lineage, or a known variant having a similar sequence (e.g., a sequence that is at least 90% identical to the given variant). In some embodiments, growth of a given variant is a function of the change in the number of subjects within a subject population who are reported as being infected with the given variant over a given time period relative to a reference infection rate (e.g., a reference infection rate determined over a defined period of time). In some embodiments, growth of a given variant is a function of the change in the proportion of a subject population infected with the given variant over a given time period relative to a reference infection rate (e.g., a reference infection rate determined over a defined period of time). In some embodiments, a growth score of a given variant can be an empirically determined by considering sequences associated with a given variant (e.g., in some embodiments including sequences associated with a lineage) that have been observed within a defined period and computing its proportion among all observed sequences at a given time relative to a reference level (e.g., its proportion determined over a defined period of time). For example, in some embodiments, for each lineage, its proportion of sequences among all observed sequences is calculated for an extended period of time (e.g., an eight-week window) and for the most recent time window (e.g., for the last 24 hours, last 48 hours, last 72 hours, last 4 days, last 5 days, last 6 days, or last week), denoted by r_{extended} and rₗₐₛₜ, respectively. The growth of the lineage is defined by their ratio r_{extended}/rₗₐₛₜ, measuring the change of the proportion. In various embodiments described herein, a growth score can be normalized such that it ranks between 0 and 100%.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Human: In some embodiments, a human is an embryo, a fetus, an infant, a child, a teenager, an adult, or a senior citizen.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Immune Escape Score”: As used herein, the term “immune escape score” refers to a measure of a viral variant's ability to escape detection and/or neutralization by antibodies (e.g., neutralization antibodies generated by a patient that has previously been infected and/or vaccinated against a reference sequence). In some embodiments, determination of an immune escape score comprises calculating a semantic change score (e.g., a semantic change score determined using a method disclosed herein). In some embodiments, determination of an immune escape score comprises calculation of an epitope alteration score (e.g., using one of the methods described herein). In some embodiments, the immune escape score is determined using a combination of an epitope alteration score and a semantic change score. In some embodiments, the immune escape score is an average of the epitope alteration score and the semantic change score.

“Infectivity Score” or “Fitness Prior Score”: as used interchangeably herein, the term “infectivity score” or “fitness prior score” is a measure of a viral variant's evolutionary fitness, and is a function of the efficiency with which a virus replicates and/or the efficiency with which a virus infects host cells. In some embodiments, calculation of a fitness prior score comprises determining one or more of a log-likelihood score, a viral polypeptide receptor binding score, and/or a growth score. In some embodiments, a fitness prior score is determined by referencing each of a log-likelihood score, a viral polypeptide receptor binding score, and a growth score.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

In vitro: The term “in vitro” as used herein refers to events that occur in an artificial environment, e.g., in a test tube or reaction vessel, in cell culture, etc., rather than within a multi-cellular organism.

In vitro transcription: As used herein, the term “in vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but not are limited to mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Log-likelihood: As used herein, the term “log-likelihood” refers to a measure of the existence probability of a variant polypeptide sequence, which has been determined using natural language learning algorithms. In some embodiments, log-likelihood can be determined using a transformer model. In some embodiments, log-likelihood can be determined without a reference sequence. In some embodiments, log-likelihood is a transformer-derived log-likelihood without reference. The higher the log-likelihood of a variant, the more probable the variant is to occur from a language model perspective. In various embodiments described herein, log-likelihood can be normalized such that it ranks between 0 and 100%. In some embodiments, a log-likelihood measures how log-likelihood of a variant polypeptide sequence compares to the entire population of known variants. In some embodiments, a log-likelihood measures how log-likelihood of a variant polypeptide sequence compares to other variants with similar mutational loads (“conditional log-likelihood”). Such conditional log-likelihood is particularly useful for assessing variants with high mutation counts (e.g., at least 30 or more, including, e.g., at least 40, at least 50, at least 60, at least 70, or more mutation counts).

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, a nanoparticle has a diameter of less than 80 nm as defined by the National Institutes of Health. In some embodiments, a nanoparticle comprises one or more enclosed compartments, separated from the bulk solution by a membrane, which surrounds and encloses a space or compartment.

Nucleic acid: As used herein, the term “nucleic cad” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Pareto Score: As used herein, the term “Pareto score” refers to a measure of a variant's fitness and ability to escape an immune response. In some embodiments, a Pareto score comprises a combination of an immune escape score (e.g., as described herein) and a fitness prior score (e.g., as described herein). In some embodiments, a Pareto score captures the relative evolutionary advantage of a given strain. In some embodiments, such a Pareto score can be determined as described in the Examples. In some embodiments, a Pareto score is an optimality score, which, for example in some embodiments ranks a variant relative to other sequences, e.g., ones that are observed in a population. A high Pareto score at a given time for a specific lineage indicates that fewer variants have higher scores for fitness prior and immune escape at that time. As a Pareto score in some embodiments is a ranking system, and fitness prior and immune escape scores incorporated therein can change as new data are acquired, the Pareto score for a given variant can change over time. As used herein, in some embodiments, Pareto optimality is defined over a set of lineages. In some embodiments, lineages are Pareto optimal within a set if there are no lineages in the set with higher immune escape and higher fitness prior scores. In some embodiments, a Pareto score is a measure of the degree of Pareto optimality. For example, in some embodiments, lineages with the highest Pareto score are Pareto optimal; and lineages with the second-best Pareto score would be Pareto optimal, if the Pareto optimal lineages were removed from the set, and so on.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes a viral infection (e.g., a SARS-CoV-2 infection). In some embodiments, the patient is receiving or has received certain therapy to diagnose and/or to treat a disease, disorder, or condition.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to an active agent, formulated together with one or more pharmaceutically acceptable carriers. In some embodiments, active agent is present in a unit dose amount that is appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical grade: The term “pharmaceutical grade” as used herein refers to standards for chemical and biological drug substances, drug products, dosage forms, compounded preparations, excipients, medical devices, and dietary supplements, established by a recognized national or regional pharmacopeia (e.g., The United States Pharmacopeia and The Formulary (USP-NF)).

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Pure or Purified: As used herein, an agent or entity is “pure” or “purified” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure in a preparation.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is an mRNA. In some embodiments where an RNA is an mRNA, a RNA typically comprises at its 3′ end a poly(A) region. In some embodiments where an RNA is an mRNA, an RNA typically comprises at its 5′ end an art-recognized cap structure, e.g., for recognizing and attachment of an mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Recombinant: as used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Recovering: as used herein, refers to the process of rendering an agent or entity substantially free of other previously-associated components, for example by isolation, e.g., using purification techniques known in the art. In some embodiments, an agent or entity is recovered from a natural source and/or a source comprising cells.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Room temperature: As used herein, the term “room temperature” refers to an ambient temperature. In some embodiments, a room temperature is about 18° C.-30° C., e.g., about 18° C.-25° C., or about 20° C.-25° C., or about 20-30° C., or about 23-27° C. or about 25° C.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, spleen, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Semantic Change: As used herein, the term “semantic change” refers to a measure of a functional change of a viral polypeptide of a variant (e.g., in some embodiments a viral polypeptide that interacts with a host cell receptor and/or is otherwise involved in host cell entry) with respect to at least one or a plurality of (e.g., at least two, at least three, at least four, or more) reference viral polypeptide(s) (e.g., in some embodiments reference viral polypeptides of wild type species and/or known variants, e.g., of the same lineage) from the language model perspective. In some embodiments, a semantic change is a measure of a functional change of a viral polypeptide of a variant (e.g., in some embodiments a viral polypeptide that interacts with a host cell receptor and/or otherwise involved in host cell entry) with respect to a plurality of (e.g., at least two or more) reference viral polypeptide(s) (e.g., in some embodiments reference viral polypeptides of wild type species and/or known variants, e.g., of the same lineage) from the language model perspective. In some embodiments, a relevant language model can comprise Transformer-derived embedding differences (e.g., as described herein) with respect to at least one or a plurality of (e.g., at least two, at least three, at least four, or more) reference viral polypeptide(s) (e.g., in some embodiments reference viral polypeptides of wild type species or known variants, e.g., of the same lineage). In some embodiments, a semantic change score can be computed using L1 norm. In some embodiments, a sematic change score can be computed using L2 norm (also known as Euclidean norm). In some embodiments, semantic change describes how different a variant is with regard to an underlying statistical model (e.g., in some embodiments a large machine learning model fine-tuned on viral protein sequences observed until a given time point). In some embodiments, semantic change score depends on sequences observed, and thus the semantic change score may change over time, as an underlying model is trained on new variant sequences and/or reference sequences. In some embodiments, a semantic change score is determined for a variant Spike polypeptide from SARS-Co-V-2 as described herein. In various embodiments described herein, a semantic change score can be normalized such that it ranks between 0 and 100%.

Single Nucleotide Polymorphism (SNP): As used herein, the term “single nucleotide polymorphism” or “SNP” refers to a particular base position in the genome where alternative bases are known to distinguish one allele from another. In some embodiments, one or a few SNPs and/or CNPs is/are sufficient to distinguish complex genetic variants from one another so that, for analytical purposes, one or a set of SNPs and/or CNPs may be considered to be characteristic of a particular variant, trait, cell type, individual, species, etc., or set thereof. In some embodiments, one or a set of SNPs and/or CNPs may be considered to define a particular variant, trait, cell type, individual, species, etc., or set thereof.

Stable: The term “stable,” when applied to nucleic acids and/or compositions comprising nucleic acids, e.g., encapsulated in lipid nanoparticles, means that such nucleic acids and/or compositions maintain one or more aspects of their characteristics (e.g., physical and/or structural characteristics, function, and/or activity) over a period of time under a designated set of conditions (e.g., pH, temperature, light, relative humidity, etc.). In some embodiments, such stability is maintained over a period of time of at least about one hour; in some embodiments, such stability is maintained over a period of time of about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, such stability is maintained over a period of time within the range of about one (1) day to about twenty-four (24) months, about two (2) weeks to about twelve (12) months, about two (2) months to about five (5) months, etc. In some embodiments, such stability is maintained under an ambient condition (e.g., at room temperature and ambient pressure). In some embodiments, such stability is maintained under a physiological condition (e.g., in vivo or at about 37° C. for example in serum or in phosphate buffered saline). In some embodiments, such stability is maintained under cold storage (e.g., at or below about 4° C., including, e.g., −20° C., or −70° C.). In some embodiments, such stability is maintained when nucleic acids and/or compositions comprising the same are protected from light (e.g., maintaining in the dark).

As an example, in some embodiments, the term “stable” is used in reference to a nanoparticle composition (e.g., a lipid nanoparticle composition). In such embodiments, a stable nanoparticle composition (e.g., a stable nanoparticle composition) and/or component(s) thereof maintain one or more aspects of its characteristics (e.g., physical and/or structural characteristics, function(s), and/or activity) over a period of time under a designated set of conditions. For example, in some embodiments, a stable nanoparticle composition (e.g., a lipid nanoparticle composition) is characterized in that average particle size, particle size distribution, and/or polydispersity of nanoparticles is substantially maintained (e.g., within 10% or less, as compared to the initial characteristic(s)) over a period of time (e.g., as described herein) under a designated set of conditions (e.g., as described herein). In some embodiments, a stable nanoparticle composition (e.g., a lipid nanoparticle composition) is characterized in that no detectable amount of degradation products (e.g., associated with hydrolysis and/or enzymatic digestion) is present after it is maintained under a designated set of conditions (e.g., as described herein) over a period of time.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein, the term “substantial identify” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition displays one or more symptoms of a disease, disorder, and/or condition and/or has been diagnosed with the disease, disorder, or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present disclosure, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Three prime untranslated region (3′ UTR): As used herein, the terms “three prime untranslated region” or “3′ UTR” refer to the sequence of an mRNA molecule that begins following the stop codon of the coding region of an open reading frame sequence. In some embodiments, the 3′ UTR begins immediately after the stop codon of the coding region of an open reading frame sequence. In other embodiments, the 3′ UTR does not begin immediately after stop codon of the coding region of an open reading frame sequence.

Threshold level: As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition and/or for delaying onset or decreasing rate of development or worsening of one or more features of a disease, disorder and/or condition.

Vaccination: As used herein, the term “vaccination” refers to the administration of a composition intended to generate an immune response, for example to a disease (e.g., to a viral epitope). In some embodiments, vaccination can be administered before, during, and/or after development of a disease. In some embodiments, vaccination includes multiple administrations, appropriately spaced in time, of a vaccinating composition.

Viral polypeptide receptor binding score: As used herein, the term “viral polypeptide receptor binding score” refers to a measure of binding affinity between a viral polypeptide that plays a role in host recognition and/or host cell entry, and a corresponding host protein with which the viral polypeptide interacts to recognize and/or enter a host cell. In some embodiments, a viral polypeptide receptor binding score is determined in silico. In some embodiments, a viral polypeptide receptor binding score can be determined using a conformational sampling algorithm. In some embodiments, a viral polypeptide receptor binding score can be determined using structures that have been optimized using a probabilistic optimization algorithm (for example, in some embodiments a variant of simulated annealing, aiming to overcome local energy barriers and follow a kinetically accessible path toward an attainable deep energy minimum with respect to a knowledge-based, protein-oriented potential). In some embodiments, a viral polypeptide receptor binding score can be calculated using the change in solvent accessible surface area (SAAS) of a viral polypeptide in a complexed state (e.g., a bound state) and a non-complexed state (e.g., a non-bound state). In some embodiments, a viral polypeptide receptor binding score can be determined by calculating the change in energy of the complexed (e.g., bound) and non-complexed (e.g., non-bound) structures of a viral polypeptide and its cognate host receptor. In some embodiments, change in binding energy can be estimated by differences in Gibbs free energy between bound and unbound states. In various embodiments described herein, a viral polypeptide receptor binding score can be normalized such that it ranks between 0 and 100%. In some embodiments, a viral polypeptide receptor binding score can be calculated in silico, e.g., by calculating the change in Gibbs Free Energy, or the change in solvent accessible surface area in the bound and unbound states. In some embodiments, a viral polypeptide receptor binding score can be calculated using in vitro binding data (e.g., using a dissociation constant, K_{D}, or an association rate, kₒₙ). In some embodiments, such in vitro binding data can be determined methods known in the art, including, e.g., but not limited to biolayer interferometry (BLI) and/or surface plasmon resonance (SPR).

ACE2 Binding Score: As used herein, the term “ACE2 binding score” is a viral polypeptide receptor binding score (as described herein), wherein the viral polypeptide receptor is angiotensin-converting enzyme 2 (ACE2). An “ACE2 binding score” is a measure of binding affinity between an S protein of a coronavirus (e.g., SARS-CoV-2) or an immunogenic fragment of the S protein (e.g., the RBD domain) and the ACE2 protein. In some embodiments, an ACE2 binding score can be calculated in silico, e.g., by calculating the change in Gibbs Free Energy, or the change in solvent accessible surface area in the bound and unbound states. In some embodiments, an ACE2 binding score can be calculated using in vitro binding data (e.g., using a dissociation constant, K_{D}, or an association rate, kₒₙ). In some embodiments, such in vitro binding data can be determined methods known in the art, including, e.g., but not limited to biolayer interferometry (BLI) and/or surface plasmon resonance (SPR).

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles). In some embodiments, in the context of SARS-CoV-2, “wild-type” refers to the Wuhan variant.

### DETAILED DESCRIPTION OF CERTAIN EMBODIMENTS

The present disclosure, among other things, provides technologies for identifying, characterizing, and/or monitoring sequences of a variant of a reference infectious agent (e.g., but not limited to viral variants, for example in some embodiments SARS-CoV-2 variants) for transmissibility factors and/or immune escape potential, and/or for detecting and/or monitoring variants in environmental or biological samples, and/or for designing, preparing, and/or administering vaccines for such variants.

Variants differ from reference agents (e.g., reference infectious agents or reference vaccine agents) by amino acid sequence alteration(s) (e.g., one or more substitutions, additions, deletions, and/or inversions of a single amino acid or of a set of adjacent amino acids).

In some embodiments, provided technologies are relevant to variants that arise and/or spread in a particular geographic location or within a particular community of contacts. In some embodiments, provided technologies are relevant to variants with greater infectivity and/or morbidity than a relevant reference variant. In some embodiments, provided technologies are relevant to so-called “escape” variants, able to evade an immune response to a reference agent. In some such embodiments, such immune response occurs or has occurred as a result of infection with a reference agent; in some such embodiments, such immune response occurs or has occurred as a result of immunization with a reference agent. For example, in some embodiments, such a variant can be an escape variant that is able to evade immunity that subjects acquire through vaccines and/or prior infections.

In particular embodiments, the present disclosure provides results of an in silico approach combining (1) modeling of one or more structural feature(s) of a viral protein that may be involved in a process of virus invasion of a host, and (ii) one or more protein transformer language models on such viral protein sequences to reliably rank variants (e.g., in some embodiments currently circulating variants and/or previously circulating variants) for transmissibility factors and/or immune escape potential. In some embodiments, modeling of one or more structural feature(s) of a viral protein comprises (i) determining impact of amino acid sequence alteration(s) on viral fitness (e.g., efficacy of viral cell entry, and/or its structure and/or function), which is indicative of infectivity or transmissibility potential; and (ii) determining likelihood of a mutated epitope to evade neutralization by an immune system, which is indicative of immune escape potential.

The present disclosure, among other things, recognizes the source of problems that are associated with the “grammaticality” approach (e.g., as described in Hie et al., Science 371 (2021)284-288) and provides a different approach that provide certain particular advantages, including for example by using a “log-likelihood” approach. The higher the log-likelihood of a variant, the more probable is the variant to occur from a language model perspective. The present disclosure, among other things, appreciates that the log-likelihood metric supports substitutions, insertions and deletions without requiring a reference.

The present disclosure, among other things, also recognizes that values of log-likelihood tend to diminish with an increasing number of mutations, which can result in over-emphasis of variants with low mutation counts, and appreciates the importance of introducing a conditional log-likelihood score for variants with high mutational loads (e.g., at least 30 mutations, at least 40 mutations, at least 50 mutations, at least 60 mutations, at least 70 mutations, and more), which measures how the log-likelihood of a variant compares to other variants with similar mutational loads (e.g., as described herein), as opposed to the entire population of known variants. For example, a variant B.1.1.529 (Omicron) with a high mutational load might be perceived by raw log-likelihood (i.e., relative to the entire population of all other variants) as a low risk variant, but relative to a sub-population of variants with a similar number of mutations, Omicron clearly stands out as a high risk variant with a high conditional log-likelihood.

In some embodiments, modeling with one or more protein transformer languages comprises, based on machine learning, determination of a semantic change score, which indicates predicted variation in one or more biological functions between a variant and a reference viral polypeptide; and/or determination of log-likelihood or conditional log-likelihood, which is a measure to characterize a variant polypeptide.

The present disclosure, among other things, provides an insight that growth of certain variants can change over time and/or geographical locations and thus in some embodiments it is desirable to include such metric to determine infectivity potential of a given variant. The present disclosure, among other things, also appreciates that because there are changes over time, a single variant as determined by methods described herein does not necessarily have a single immune escape or infectivity score.

Among other things, in some embodiments, the present disclosure provides an insight that transmissibility and immune escape metrics can be combined for an automated Early Warning System (EWS) that is capable of evaluating new variants in such short period of time that enables risk monitoring of variant lineages in near real time. In some embodiments, such an EWS can be trained on large datasets of sequence data (e.g., comprising genomic sequences and/or protein sequences) of known infectious agents (e.g., viral agents of interest, for example in some embodiments SARS-CoV-2, as well as known variants thereof) in an unsupervised manner and can predict variants that may arise, or may be prevalent or rapidly spreading in a certain region. In some particular embodiments, the present disclosure provides EWS technologies for detection and/or characterization of viral variants, and specifically SARS-CoV-2 variants. In some embodiments, such technologies can be useful for predicting which SARS-CoV-2 variants are likely to be variants of interest.

In some embodiments, provided technologies may be or include one or more immunogenic compositions (e.g., vaccines) that deliver a variant sequence comprising one or more amino acid substitutions identified using technologies described herein and/or methods (e.g., of making, using, assessing, etc.) such immunogenic compositions. In some embodiments, variants of interest may be potential escape variants (e.g., variants with an increased likelihood of being able to evade a subject's immune response). In some embodiments, provided technologies can be useful for designing and/or manufacturing immunogenic compositions (e.g., vaccines) directed to a variant of a reference infectious agent (e.g., but not limited to viral variants, for example, in some embodiments, SARS-CoV-2 variants). In some embodiments, provided technologies may be useful for prevention and/or treatment of an infection associated with a viral protein of interest.

### Methods of Identifying Variants of Interest

In some embodiments, the present disclosure provides methods for assessing the risk for a variant of a reference viral polypeptide. In some embodiments, a variant that is found to have an elevated risk using the methods disclosed herein has an increased likelihood of spreading in a population. In some embodiments, a variant that is found to have an elevated risk using a method disclosed herein has an increased likelihood of spreading in a population, an increased likelihood of infecting more subjects in a population, and/or an increased likelihood of infecting a larger fraction of subjects in the population.

In some embodiments, the present disclosure provides a method for assessing risk for a variant of a reference viral polypeptide, the method comprising: providing an amino acid sequence of the variant polypeptide, which comprises one or more amino acid modifications relative to the reference viral polypeptide; modeling one or more structural features of the variant polypeptide that are involved in viral invasion of a host; determining, based on genomic data associated with the viral polypeptide, distance of each of the one or more amino acid modifications relative to the corresponding amino acids in the reference viral polypeptide to determine probability of observing each amino acid modification; and designating the variant polypeptide as a variant with elevated risk when the variant polypeptide is characterized in that: (a) it has an immune escape score that satisfies a pre-determined immune escape threshold indicating likelihood of the variant polypeptide to be detected and neutralized by antibodies; and (b) it has an infectivity score that satisfies a pre-determined infectivity threshold indicating likelihood of the variant polypeptide to a relevant host receptor.

In some embodiments, the present disclosure provides a method for assessing risk for a plurality of variants of a reference viral polypeptide, the method comprising: providing a plurality of amino acid sequences of variant polypeptides, wherein each of the variant polypeptides comprises one or more amino acid modifications relative to the reference viral polypeptide; ascertaining, for each of the variant polypeptides, an immune escape score (indicative of likelihood of its detection and neutralization by antibodies) and an infectivity score (indicative of likelihood of its binding to a relevant host receptor) by performing the following processes: modeling one or more structural features of each variant polypeptide that are involved in viral invasion of a host; determining, based on genomic data associated with the viral polypeptide, distance of each of the one or more amino acid modifications relative to the corresponding amino acids in the reference viral polypeptide to determine the probability of observing each amino acid modification; ranking risk of the variant polypeptides in the plurality by referencing respective combined scores of the immune escape score and the infectivity score; and designating a variant polypeptide as a variant polypeptide with elevated risk when its combined score is ranked higher than that of at least one other variant polypeptide in the plurality.

In some embodiments, a variant polypeptide is designated as elevated risk when (a) it has an immune escape score that satisfies a pre-determined immune escape threshold indicating likelihood of the variant polypeptide to be detected and neutralized by antibodies; and/or (b) it has an infectivity score that satisfies a pre-determined infectivity threshold indicating likelihood of the variant polypeptide to a relevant host receptor.

In some embodiments, a variant polypeptide is designated as elevated risk when (a) it has an immune escape score that satisfies a pre-determined immune escape threshold indicating likelihood of the variant polypeptide to be detected and neutralized by antibodies; and/or (b) it has an infectivity score that satisfies a pre-determined infectivity threshold indicating likelihood of the variant polypeptide to a relevant host receptor.

In some embodiments, a variant polypeptide is designated as elevated risk when (a) it has an immune escape score that is higher than the immune escape score of other variant polypeptides that are prevalent at the time of assessment (e.g., a score that is in the top 50% of sequences assessed, 40% of sequences assessed, 30% of sequences assessed, 20% of sequences assessed, 15% of sequences assessed, 10% of sequences assessed, or 5% of sequences assed), and/or (b) it has an infectivity score that is higher than the infectivity score of other variant polypeptides that are prevalent at the time of assessment (e.g., a score that is in the top 50% of sequences assessed, 40% of sequences assessed, 30% of sequences assessed, 20% of sequences assessed, 15% of sequences assessed, 10% of sequences assessed, or 5% of sequences assed), and/or a combination of immune escape score and infectivity score that is higher than those of other variant polypeptides that are prevalent at the time of assessment (e.g., a combined score that is in the top 50% of sequences assessed, 40% of sequences assessed, 30% of sequences assessed, 20% of sequences assessed, 15% of sequences assessed, 10% of sequences assessed, or 5% of sequences assed). In some embodiments, each of the variant polypeptides in a plurality of polypeptides share an overall amino acid sequence identity of at least 80% with each other (e.g., at least 85%, at least 90%, at least 95%, at least 96%, at least 97%, at least 98%, or at least 99% with each other). In some embodiments, each of the variant polypeptides in a plurality of polypeptides have an overall amino acid sequence identity of at least 80% with a reference polypeptide (e.g., at least 85%, at least 90%, at least 95%, at least 96%, at least 97%, at least 98%, or at least 99% with the reference polypeptide). In some embodiments, variant polypeptides that have been designated as having an elevated risk using technologies described herein are considered as “High Risk Variants” (HRV). In some embodiments, variant polypeptides that have been designated as having an elevated risk using technologies described herein are considered as “Variants of concern” (VOC). In some embodiments, variant polypeptides that have been designated as having an elevated risk using technologies described herein are considered as “Variants of Interest” (VOI). In some embodiments, variant polypeptides that have been designated as having an elevated risk using technologies described herein are considered as “Variants under Monitoring” (VUM).

In some embodiments, the viral polypeptide is a SARS-CoV-2 Spike polypeptide. In some embodiments, the SARS-CoV-2 variant is an engineered variant.

Without wishing to be bound by any theory, the likelihood of a variant spreading rapidly in a patient population is a function of its infectivity (for example, its ability to replicate rapidly and spread from subject to subject) and/or its ability to escape immune response in subjects. In some embodiments, the methods disclosed herein comprise determining the likelihood of a variant being able to evade immune responses in subjects who have been previously vaccinated against the reference virus or a variant thereof. In some embodiments, the methods disclosed herein comprise determining the likelihood of a variant being able to evade immune responses in subjects who have been previously infected with the reference virus or a variant thereof.

In some embodiments, the detection methods disclosed herein can be used to assess the risk of coronavirus variants (e.g., SARS-CoV-2 variants). Coronaviruses are enveloped, positive-sense, single-stranded RNA ((+) ssRNA) viruses. They have the largest genomes (26-32 kb) among known RNA viruses and are phylogenetically divided into four genera (α, β, γ, and δ), with betacoronaviruses further subdivided into four lineages (A, B, C, and D). Coronaviruses infect a wide range of avian and mammalian species, including humans. Some human coronaviruses generally cause mild respiratory diseases, although severity can be greater in infants, the elderly, and the immunocompromised. Middle East respiratory syndrome coronavirus (MERS-CoV) and severe acute respiratory syndrome coronavirus (SARS-CoV), belonging to betacoronavirus lineages C and B, respectively, are highly pathogenic. Both viruses emerged into the human population from animal reservoirs within the last 15 years and caused outbreaks with high case-fatality rates. The outbreak of severe acute respiratory syndrome coronavirus-2 (SARS-CoV-2) that causes atypical pneumonia (coronavirus disease 2019; COVID-19) has raged in China since mid-December 2019, and has developed to be a public health emergency of international concern. SARS-CoV-2 (MN908947.3) belongs to betacoronavirus lineage B. It has at least 70% sequence similarity to SARS-CoV.

In general, coronaviruses have four structural proteins, namely, envelope (E), membrane (M), nucleocapsid (N), and spike (S). The E and M proteins have important functions in the viral assembly, and the N protein is necessary for viral RNA synthesis. The critical glycoprotein S is responsible for virus binding and entry into target cells. The S protein is synthesized as a single-chain inactive precursor that is cleaved by furin-like host proteases in the producing cell into two noncovalently associated subunits, S1 and S2. The S1 subunit contains the receptor-binding domain (RBD), which recognizes the host-cell receptor. The S2 subunit contains the fusion peptide, two heptad repeats, and a transmembrane domain, all of which are required to mediate fusion of the viral and host-cell membranes by undergoing a large conformational rearrangement. The S1 and S2 subunits trimerize to form a large prefusion spike.

The S precursor protein of SARS-CoV-2 can be proteolytically cleaved into S1 (685 aa) and S2 (588 aa) subunits. The S1 subunit consists of the receptor-binding domain (RBD), which mediates virus entry into sensitive cells through the host angiotensin-converting enzyme 2 (ACE2) receptor.

In some embodiments, the methods disclosed herein comprise modeling one or more structural features of the variant polypeptide that is being assessed, wherein the one or more structural features are involved in viral invasion of a host. In some embodiments, modeling comprises determining the binding affinity in silico between the variant polypeptide and one or more host features (e.g., cell surface proteins) with which the variant polypeptide can associate with prior to entering a host cell. As will be understood by one of skill in the art, binding affinity can be determined in silico using appropriate methods known in the art. In some embodiments, binding affinity can be determined in silico using the median difference in solvent accessible surface between bound and unbound states of the variant polypeptide. In some embodiments, binding affinity can be characterized by using potential energy of the binding interaction. In some embodiments, binding affinity can be characterized by using Gibbs free energy of the binding interaction between a variant polypeptide and a cognate binding receptor. For example, in some embodiments, binding affinity can be computed as the change in Gibbs free energy between the bound and the unbound state of a variant polypeptide with a cognate binding receptor. In some embodiments, the variant polypeptide is the S protein from SARS-CoV-2. In some embodiments, the S protein is modeled. In some embodiments, the receptor binding domain (RBD) of the S protein is modeled. In some embodiments, a portion of the S protein that interacts with the ACE2 receptor is modeled. In some embodiments, the binding affinity between the variant protein or a portion thereof (e.g., the RBD domain of the S protein) and the host component (e.g., the ACE2 receptor) is determined in silico, through repeated, fully flexible docking experiments, allowing for unbiased sampling of the binding landscape. In some embodiments, the in silico determined binding affinity between the S protein or the RBD and the ACE2 receptor is used to calculate an ACE2 biding score. In some embodiments, the methods disclosed herein comprise comparing the binding affinity determined for the variant polypeptide with that determined for the reference polypeptide.

In some embodiments, the methods disclosed herein comprise experimentally measuring the binding affinity between the variant polypeptide that is being assessed and one or more host features (e.g., cell surface proteins) with which the variant polypeptide can associate with prior to entering a host cell. In some embodiments, the binding affinity is determined in vitro. As will be understood by one of skill in the art, binding affinity can be determined using appropriate methods known in the art. Exemplary methods for measuring binding affinity include ELISAs, gel-shift assays, pull-down assays, equilibrium dialysis, analytical ultracentrifugation, surface plasmon resonance, isothermal titration calorimetry, and spectroscopic assays. In some embodiments, binding affinity can be determined in vitro using Surface Plasmon resonance (SPR). In some embodiments, the risk associated with a variant polypeptide can first be estimated using a method that uses an in silico determined binding score, and then verified using a method that uses an in vitro determined binding affinity. In some embodiments, the variant polypeptide is the S protein from SARS-CoV-2. In some embodiments, the variant polypeptide is the receptor binding domain (RBD) of the S protein. In some embodiments, the methods disclosed herein comprise comparing the binding affinity determined for the variant polypeptide with that determined for the reference polypeptide/.

In some embodiments, the risk of a variant polypeptide is assessed by determining the likelihood that the sequence of the variant polypeptide would occur, wherein this likelihood is determined by comparison to sequences of the reference polypeptide and its variants that have been previously determined, optionally in combination with comparison to other known polypeptide sequences. In some embodiments, this comparison is performed using a machine learning algorithm, wherein the machine learning algorithm has been trained on sequences of the reference polypeptide and its variants, and optionally wherein the algorithm has been further trained using a broader database of polypeptide sequences. In some embodiments, the machine learning algorithm uses a learning language model. In some embodiments, the learning language model calculates a distance between a reference polypeptide and the variant polypeptide, where a larger distance indicates a lower probability that the sequence would arise naturally, and results in an increased escape score.

In some embodiments, the machine learning algorithms used in the methods disclosed herein use a recurrent neural networks used (e.g., as used in Hie et al., 2021). In some embodiments, the machine learning algorithms use attention-based models, namely transformers (e.g., as used in Vaswani et al., 2017), rather than recurrent neural networks, hence replacing the auto-regressive way of training the model (Hie et al., 2021) by the BERT (Bidirectional Encoder Representations from Transformers) protocol. Even though the GISAID dataset contains hundreds of thousands of protein sequences, its volume remains limited compared to more general protein data banks such as the UniProt50 database that includes hundreds of millions of protein sequences (UniProt Consortium, 2019). Accordingly, in some embodiments, the machine learning algorithms are first pre-trained over a large collection of varied proteins (e.g., the proteins included in UniProt50) and then fine-tuned over sequences of the variant polypeptide (e.g., S protein sequences). In some embodiments, the transformer model is re-trained on a regular basis, so as to incorporate the latest sequence information. In some embodiments, the transformer model is updated once every 6 months, once every 5 months, once every 4 months, once every 3 months, once every 2 months, once every month, once biweekly, or once a week on average. In some embodiments, the transformer model is re-trained every month on all the S protein variants registered in GISAID (122,466 unique S sequences on 3rd of September 2021 vs. 4,172 S sequences in Hie et al. (Hie et al., 2021)).

In some embodiments, the semantic change calculation is computed to estimate the change of a variant sequence relative to one or more sequences of a variant protein. In some embodiments, the semantic change calculation is computed to estimate the change relative to one or more reference sequences that are prevalent at the time of assessing the new variant. In some embodiments, the semantic change calculation is computed to estimate the change relative to the first sequence determined for a virus. In some embodiments, the semantic change calculation is computed to estimate change relative to the first sequence determined for a virus and one or more variants. In some embodiments, the semantic change calculation is computed to estimate change relative to the first sequence determined for a virus and one or more variants that are prevalent at the time of assessing the new variant. In some embodiments, the semantic change calculation is computed to estimate the change with respect to the wild type SARS-CoV-2 S protein sequence and from the D614G mutation to take into account that the D614G mutant has largely replaced the Wuhan strain. In some embodiments, a transformer model is used to calculate the log-likelihood of an input sequence: the likelihood of occurrence of a given input sequence. The higher the log-likelihood of a variant, the more probable is the variant to occur from a language model perspective. In particular, the log-likelihood metric supports substitutions, insertions and deletions without requiring a reference.

In some embodiments, experimental data (including, e.g., in vitro data) can be used to validate immune escape scores determined in silico. For example, in some embodiments, in vitro pseudovirus neutralization test (pVNT) assays can be used to validate immune escape scores determined in silico (e.g., semantic change score and/or epitope score). As will be understood by skilled artisans, other appropriate surrogate virus neutralization assays can be used to validate immune escape scores determined in silico. By way of example only, in some embodiments, a surrogate virus neutralization assay based on antibody-mediated blockage of interaction between a viral polypeptide receptor and a target variant polypeptide (e.g., a SARS-CoV-2 surrogate virus neutralization test based on antibody-mediated blockage of ACE2-spike protein-protein interaction as described in Tan et al. Nature Biotechnology (2020) 38: 1073-1078 can be used to validate immune escape scores determined in silico. In some embodiments, the cross-neutralizing effect of sera derived from patients who have been vaccinated against the reference sequence and/or who have previously been infected with and recorded from a virus having the reference sequence. In some embodiments, the sera is derived from patients who have been previously infected with SARS-CoV-2. In some embodiments, the sera is derived from patients who have been previously vaccinated against SARS-CoV-2. In some embodiments, sera is assessed against viral particles from another virus, who have been altered to express the variant of interest. In some embodiments, sera is assessed against viral particles from an innocuous virus, who have been altered to express the variant of interest. In some embodiments, sera is assessed against vesicular stomatitis virus (VSV)-SARS-CoV-2-S pseudoviruses bearing the spike protein of the variant of interest. In some embodiments, both the epitope score and the semantic change score correlate positively with the calculated 50% pseudovirus neutralization titer (pVNT50) reduction. In some embodiments, the average of both in silico scores exhibits a correlation with the observed reduction in neutralizing titers.

In some embodiments, the methods disclosed herein comprise determining an immune escape score, wherein the immune escape score is a measure of the variant's ability to evade an immune response in a subject. In some embodiments, the immune escape score is a measure of the variant polypeptide's ability to avoid detection and/or neutralization by antibodies that detect and neutralize the reference polypeptide.

In some embodiments, the methods disclosed herein comprise determining an infectivity score for a variant polypeptide, wherein the infectivity score is a measure of a variant's ability to infect host subjects and/or replicate rapidly.

In some embodiments, a variant of a reference viral polypeptide is determined to be a variant with elevated risk when it has an immune escape score that satisfies (e.g., is equal to or greater than) a pre-determined immune escape threshold. In some embodiments, a variant of a reference viral polypeptide is determined to be a variant with elevated risk when it has an immune escape score that satisfies (e.g., is equal to or greater than) a pre-determined immune escape threshold. In some embodiments, a variant of a reference viral polypeptide is determined to be a variant with elevated risk when it has an immune escape score that satisfies (e.g., is equal to or greater than) a pre-determined immune escape threshold and an immune escape score that satisfies (e.g., is equal to or greater than) a pre-determined immune escape threshold.

In some embodiments, calculation of an immune escape score comprises calculation of an epitope alteration score, wherein the epitope alteration score is determined by identifying one or more sequence alterations in a variant polypeptide, and comparing the location and/or nature of the one or more sequence alterations to amino acid loci that have previously been shown to be bound by neutralizing antibodies. In some embodiments, the amino acid loci are determined using previously determined structures of the reference polypeptide in complex with neutralizing antibodies.

In some embodiments, the immune escape score is calculated using a machine learning language model. In some embodiments the machine learning language model has been trained using a database comprising sequences of the reference polypeptide and its variants. In some embodiments, the machine learning language model has been trained using a database of SARS-CoV-2 polypeptide sequences (e.g., the GISAID database). In some embodiments, the machine learning language model is first trained on a general database of protein sequences (e.g., the UniRef100 database), and then fine-tuned using a database of sequences obtained from the reference virus and variants thereof. In some embodiments, the machine learning language model is used to calculate a semantic change score for the variant polypeptide relative to the reference viral polypeptide. In some embodiments, the reference viral polypeptide is a Wuhan SARS-CoV-2 spike polypeptide or portion thereof. In some embodiments, the reference viral polypeptide is a D614G SARS-CoV-2 variant. In some embodiments, the reference viral polypeptide is derived from the variant that is most prevalent at the time of assessing the new variant.

In some embodiments, the immune escape score incorporates both the semantic change score and the epitope alteration score. In some embodiments, the immune escape score is an average of the semantic change score and the epitope alteration score.

In some embodiments, semantic change is a measure of how different a variant in question is with regard to an underlying statistical model described herein (e.g., a large machine learning model fine-tuned on viral polypeptide sequences such as, e.g., Spike protein sequences, observed until a given time point). Such semantic change score depends on sequences observed, and thus a semantic change score may change over time. On the other hand, in some embodiments, an epitope alteration score is a measure of how many distinct epitopes are evaded by the variant in question as compared to one or more reference sequences (e.g., as compared to a wild type sequence and/or a known variant sequence). In some such embodiments, an epitope alteration score can be computed based on known binding sites of antibodies, e.g., as reported in Protein Data Bank. It too changes with time with new discoveries of new antibodies against target polypeptide(s) (e.g., anti-Spike antibodies) in variants.

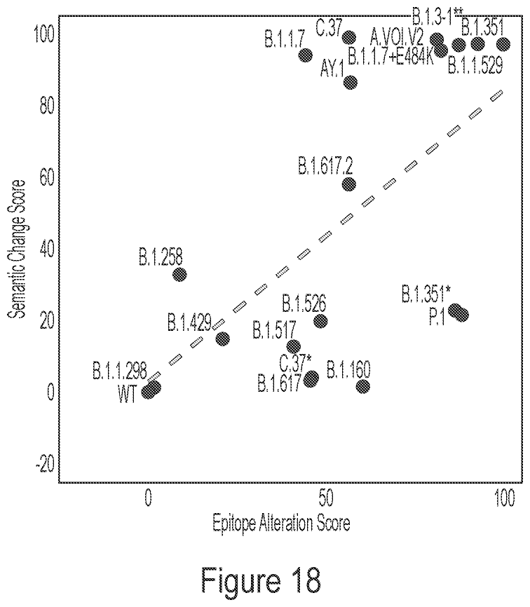

In some embodiments, a semantic change score and an epitope alteration score are collinear. In some embodiments, a semantic change score and an epitope alteration score are not collinear. In particular, as shown herein, in some embodiments, HRVs regarded as immune escaping (and denoted as VoCs, VoIs etc.) have a high semantic change score, but are diverse in terms of epitope alteration Score (see, e.g., FIG. 18).

In some embodiments, the immune escape score, the semantic change score, and/or the epitope alteration score are correlated with a pseudovirus neutralization test result. In some embodiments, the correlation is based on linear regression. In some embodiments, the correlation is based on least squares regression.

In some embodiments, a variant polypeptide is designated as a variant with elevated risk when the variant polypeptide exhibits a reduction in observed 50% pseudovirus neutralization titer (pVNT50) by at least 30% as compared to a reference viral polypeptide in a pseudovirus neutralization assay. In some embodiments, the pseudovirus neutralization assay is performed using a wild-type SARS-CoV-2 (Wuhan strain) pseudotyped VSV.

Another aspect that can contribute to infectivity of a variant, is how similar a given variant is to the other variants which have been known to grow rapidly. Effective assessment of such similarity may not be achievable by simple sequence comparison, due to epistatic interactions between sites of polymorphism, in which certain mutation combinations enhance fitness while being deleterious when they occur separately. The language model, which has experienced each individual sequence with similar frequencies in the training phase, is found to assign higher log-likelihood values to the sequences with highest observed count. In some embodiments, the methods disclosed herein use a log-likelihood of a newly observed sequence as predictive of its expected frequency in population.

The metrics discussed above may not capture the entirety of factors affecting frequency of viral variants. In some embodiments, log-likelihood or conditional log-likelihood are metrics that measure similarity to already known, rapidly increasing samples. In such embodiments, log-likelihood or conditional log-likelihood may not be able to fully assess variants which exhibit completely new sequence features, until at least one or more of such features are observed more often. Thus, in some embodiments, the methods further comprise using an infectivity metric (also known as fitness prior metric) that includes the growth rate of the variant, an empirical term of the quantified change in the fraction of observed sequences in the database that a variant in question comprises. One of the advantages of incorporating growth rate is that it may incorporate effects that may be contributed by other portions of a polypeptide variant that are outside the polypeptide sequence being assessed (e.g., proteins in SARS-CoV-2 in addition to the S protein) and/or environmental effects that are independent of the polypeptide sequence being assessed. In some embodiments, a growth score complements an ACE2 binding score which models the RBD only.

In some embodiments, the methods disclosed herein are machine learning algorithms that use neural networks (e.g., recurrent and attention-based deep neural networks). In some embodiments, the machine learning algorithms store information about protein properties at two positions inside the model once it is trained. In some embodiments, the probabilities returned by the model indicate how likely this sequence is to be natural/viable/feasible. In some embodiments, the outputs of the model's layers and notably the last layer provide a high dimensional representation for each sequence, referred to herein as embedding of the protein. The embedding of the protein contains information about the protein properties and can be used either directly or to train a classification or regression model.

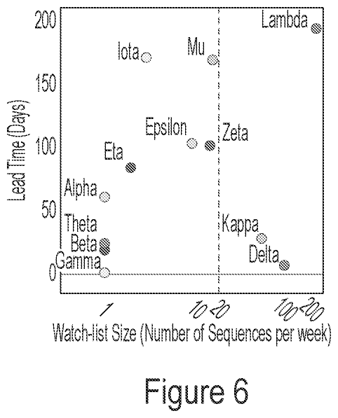

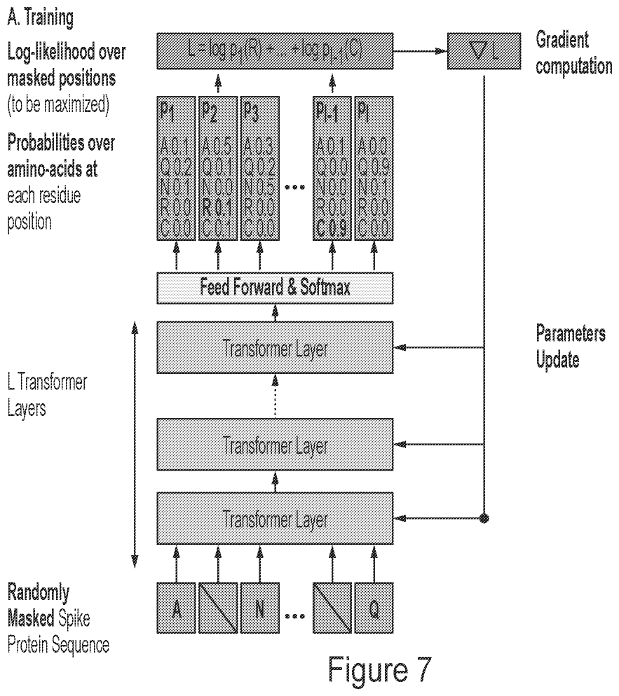

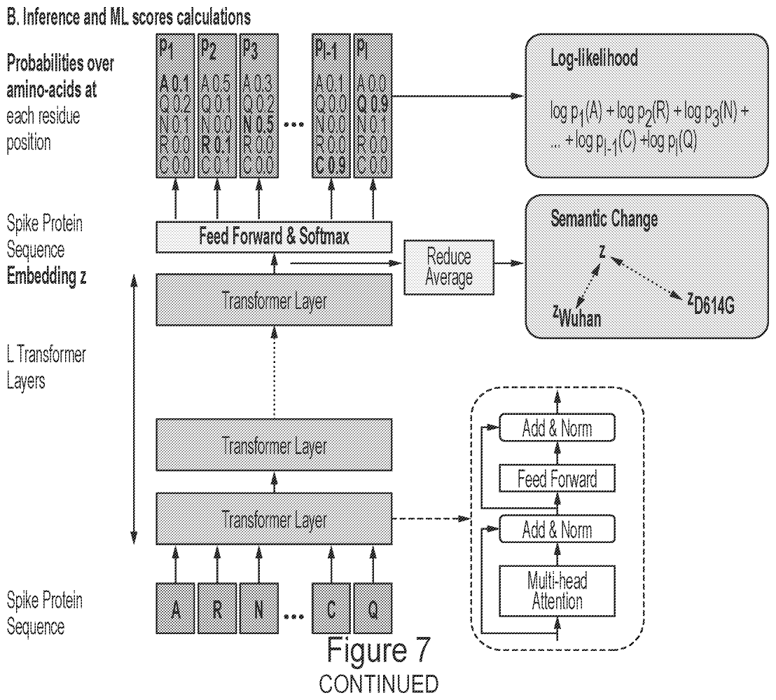

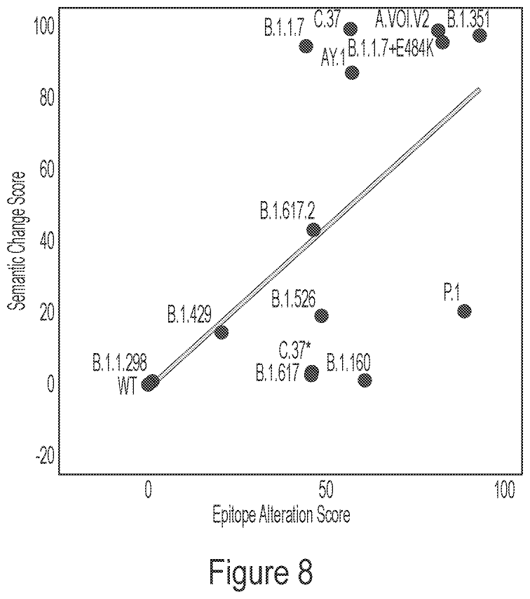

In some embodiments, the input of the models described herein comprise sequence characters corresponding to the amino acids forming the protein. In these embodiments, each amino acid can first be tokenized, i.e. mapped to their index in the vocabulary containing the 20 natural amino acids (+X), and then projected to an embedding space. The sequence of embeddings can then be fed to the Transformer model (20) consisting of a series of blocks, each composed of a self-attention operation followed by a position-wise multi-layer network (FIG. 6).

Given a large database of protein sequences, the model utilized herein can be trained using the masked language modeling objective as known in the art. Each input sequence can be corrupted by replacing a fraction of the amino acids with a special mask token. The network is can then be trained to predict the missing tokens from the corrupted sequence. In practice, for each sequence x, a set of indices i∈M are randomly sampled, for which the amino acid tokens are replaced by a mask token, resulting in a corrupted sequence \hat{x}. During pre-training the set M can be defined such that, e.g., 15% of the amino-acids in the sequence get corrupted. When corrupted an amino-acid has a fixed chance (e.g., 10%) of being replaced by another randomly selected amino-acid and fixed chance of being masked (e.g., an 80% chance of being masked). In some embodiments, during fine-tuning, these probabilities are not changed, but the percentage of corrupted amino acids may be lowered (e.g., to 3% of the amino-acids in the sequence). A probability should be selected during fine tuning that enables the model to become more accurate for spike protein sequences while keeping its performance on varied sequences from a general database of protein sequences (in embodiments where the model is first trained of a general database of sequence, and later fine-tuned using a database of sequences comprising the reference sequence and its known variants. In these embodiments, the training objective corresponds to the negative log-likelihood of the true sequence at the corrupted positions.

$${L}_{θ} ( \hat{x} \vert x ) = - {\sum }_{i ∈ M} log p ( {x}_{i} \vert \hat{x} )$$

To minimize this loss, the model must learn to identify dependencies between the corrupted and uncorrupted elements of the sequence. Consequently, the learned representations of the proteins, taken as the average of the embeddings of each amino acid, must successfully extract generic features of the biological language of proteins. These features can then be used to fine-tune the model on downstream-tasks.

In some embodiments, a transformer model, e.g., the transformer model from (Rives et al, 2021) (esm1_t34_670M_UR100), incorporated herein by reference in its entirety, can be used. In some embodiments, the training model is trained using the aforementioned procedure on a general database containing a number of sequences (e.g., trained using the UniRef100 dataset (Suzek et al., 2007), incorporated herein by reference in its entirety, and which contains greater than 277M representative sequences). In some embodiments, the pre-trained model can then be fine-tuned on a regular basis (e.g., every month) on all the reference protein variants of record at the training date.

In some embodiments, gradient descent can be used to minimize the loss function. In some embodiments, the Adam optimizer (Kingma and Ba, 2014, incorporated herein by reference in its entirety) can be used, which uses a learning rate schedule. In some embodiments, the fine-tuning stage can start with a warm-up period of, e.g., 100 mini-batches where the learning rate can be increased linearly (e.g., from 10⁻⁷ to 10⁻⁵). In some embodiments, after the warm-up period, the learning rate can be decreased, e.g., in some embodiments following 10⁻⁶\sqrt{k} where k represents the number of mini-batches.

### Data

The genomic sequences and protein sequences used to train the algorithms used herein can be collected from any database of sequences. In some embodiments the genomic sequences and protein sequences are obtained from a general database of sequences. In some embodiments, the genomic sequences and protein sequences are collected from a disease specific database (e.g., an infectious disease specific database, or a disease specific database). In some embodiments, the genomic sequences and protein sequences are collected from GISAID. For sequences that are missing amino acids, the missing amino acids can be filled in using any method known in the art, e.g., filled in using the next known amino acid and the lineage assignment using PANGOLIN (O'Toole et al., 2021). Mutations with respect to the wild type may be calculated by any method known in the art, or using any software known in the art. In some embodiments, mutations with respect to wild type can be calculated using Clustal Omega (O'Toole et al., 2021) and HH-suite (Steinegger et al., 2019), both of which are incorporated herein by reference in their entirety.

The GISAID dataset is imbalanced towards some lineages that have been more prevalent and because certain regions have performed more sequencing than others. To mitigate this bias in the dataset during training, the importance of each sequence can be weighed differently in the loss calculation. Shown below is an exemplary equation for mitigating this bias:

$${s}_{} ∝ {\sum }_{( s , l ) ∼ D} {log}_{10} ( {c}_{s} ) + {log}_{10} ( {c}_{( s , l )} ) + {log}_{10} ( {c}_{l \vert s} )$$

where the values cₛ and c_{s,l} are the numbers of occurrences in the dataset of the sequence s and the sequence-laboratory pair (s, l), respectively. The value c_{l|s} corresponds to the number of laboratories having reported sequence s, which measure the prevalence across regions of the variant.

In some embodiments, a model can exclude from training all sequences which have been observed only once in a dataset. In some embodiments, such exclusion can be useful to eliminate spurious changes, for example, due to sequencing errors, as well as samples of virus of subpar evolutionary fitness, which do not spread between patients.

Gradient descent can be used to minimize the loss function. In some embodiments, the Adam optimizer (Kingma and Ba, 2014, incorporated herein by reference in its entirety) can be used, which uses a learning rate schedule. The fine-tuning stage can start with a warm-up period of, e.g., 100 mini-batches where the learning rate can be increased linearly (e.g., from 10⁻⁷ to 10⁻⁵). After the warm-up period, the learning rate can be decreased, e.g., following 10⁻⁶ √x where x represents the number of mini-batches.

### Inference and ML Scores Calculations

Once fine-tuned, the model can be used to compute the semantic change and the log-likelihood to characterize a viral polypeptide sequence (e.g., a spike protein sequences). In some embodiments, the output of the last transformer layer can be averaged over the residues to obtain an embedding z of the protein sequence.

In some embodiments, an input sequence can be formally represented by a sequence of tokens defined as x=(x₁, . . . , xₙ) where n is the number of tokens and ∀i∈[1,n], xᵢ∈χ where χ is a finite alphabet that contains the amino-acids and other tokens such as class and mask tokens. In some embodiments, a class token is appended to all sequences before feeding them to the network, so that x₁ represents the class token, while x₂, . . . , xₙ represents the amino-acids, or masked amino-acids, in the spike protein sequence. In such embodiments, the sequence x is passed through attention layers. In these embodiments, z=(z₁, . . . , zₙ) corresponds to the output of the last attention layer where Z is the sequence embedding vector at position i.

In some embodiments, embedding vector zᵢ is a function of all input tokens (xⱼ)_{j∈[1,n]}. In contrast, in Bi-LSTM architectures, such as described in Hie et al., 2021), zᵢ would be a function of all inputs tokens except the one at the position i, (xⱼ)_{j∈[1,n],j≠i}.

In some embodiments, to represent a protein sequence through a single embedding vector, whose size does not depend on the protein sequence length, the following equation can be used

$$\overline{z} = \frac{1}{n - 1} {\sum }_{i = 2}^{n} {z}_{i} ,$$

the product of which is referred to herein as the embedding vector of the variant represented by sequence x. In the above equation, summation starts at the second position so that the class token's embedding, which is at the first position, does not contribute to the sequence embedding. In some embodiments, the embedding of a first reference strain (e.g., Wuhan strain), z̅_{wuhuan}, and the embedding of a second reference strain (e.g., D614G variant), z̅_{D614G}, can be computed once for all.

In some embodiments, the semantic change of a variant x can be computed as:

$$Δ \overline{z} = {‖ \overline{z} - {\overline{z}}_{wuhuan} ‖}_{1} + {‖ \overline{z} - {\overline{z}}_{D 614 G} ‖}_{1} ,$$

where ∥⋅∥₁ is the L1 norm. One of skill in the art, reading the present disclosure, will understand that while Wuhan and D614G sequences are used as reference sequences in the above equation, other references sequences can also be used instead.

In some embodiments, the semantic change can be computed as the sum of the Euclidean distance between the z and z_{wuhan} the Euclidean distance between z and z_{D614G}. For example, in some embodiments, the semantic change of a variant x can be computed as:

$$Δ z = {‖ z - ‖}_{2} + {‖ z - {z}_{D 614 G} ‖}_{2}$$

where ∥⋅∥₂ is the Euclidean distance (also known as L2 norm).

In some embodiments, the log-likelihood can be computed from the probabilities over the residues returned by the model. In some embodiments, it is calculated as the sum of the log-probabilities over all the positions of the spike protein amino-acids.

Given a variant's sequence s, the fine-tuned neural network provides a discrete probability distribution over all amino acids A for each position i:

$${\{ p ( {s}_{i} = ( a \vert s ) \}}_{a ∈ A}$$

where p(sᵢ=a|s) is the probability that the i-th position is amino acid a. The variant's log-likelihood metric is therefore defined as

$$l ( s ) = {\sum }_{i} log p ( {s}_{i} = {s}_{i} \vert s ) ,$$

which measures the likelihood of having the same variant given itself. In particular, the proposed log-likelihood metric supports substitution, insertion and deletion without requirement of a reference.

In some embodiments, the last attention layer output z can be transformed by a feed-forward layer and a softmax activation into a vector of probabilities over tokens at each positions p=(p₁, . . . , pₙ) where pᵢ is a vector of probabilities at position i,

$${p}_{i} = ( p ( {x}_{i} = {x}_{1} \vert x ) , … , p ( {x}_{i} = {x}_{n} \vert x ) ) .$$

In some embodiments, the log-likelihood of a variant l(x) can be computed from such probabilities. In such embodiments, the log-likelihood can be calculated as the sum of the log probabilities over all the positions of the viral polypeptide amino acids (e.g., in some embodiments Spike protein amino acids). Formally, this can be written as:

$$l ( x ) = {\sum }_{i = 2}^{n} log p ( {x}_{i} = {x}_{i} \vert x ) .$$

This above equation measures the likelihood of observing a variant sequence x according to a model (e.g., as described herein). Therefore, the more sequences in the training data that are similar to a considered variant, the higher the log-likelihood of this variant will be. The proposed log-likelihood metric supports substitution, insertion, and deletion without the requirement of a reference.

### Implementation

In some embodiments, methods disclosed herein can be implemented using the Pytorch (Paszke et al., 2019) deep learning framework. In some embodiments, model training and inferences can be performed on a high performance computing infrastructure. In some embodiments, the high performance computing infrastructure uses Nvidia A100-SXM4-40 GB GPUs. In some embodiments, the average training and inference time is <4 GPU days and <12 GPU hours, respectively, using Nvidia A100-SXM4-40 GB GPUs.

### Epitope Alteration Score

In some embodiments, an epitope alteration score described herein attempts to capture the impact of mutations in the variant in question on recognition by experimentally assessed antibodies. In some embodiments, an epitope alteration score can be computed by enumerating the number of unique epitopes involving altered positions, as measured across one or more known antibody-viral polypeptide complex structures (e.g., all known antibody antibody-Spike complex structures).

Without wishing to be bound by a particular theory, in some embodiments, an epitope alteration score as described herein is designed to emphasize the effect of mutations on highly antigenic sites of a viral polypeptide, such as in some embodiments the receptor-binding domain (RBD) of a Spike polypeptide. This allows the score to approximate the expected weight of mutations, and to ascribe importance to non-target domain mutations (e.g., non-RBD mutations), if sufficient escape potential with regard to targeting antibodies (e.g., RBD-targeting antibodies) is achieved.

### Viral Polypeptide Receptor Binding Score (e.g., ACE2 Binding Score)

In some embodiments, a viral polypeptide receptor binding score is a measure of the binding affinity between a viral polypeptide that plays a role in host recognition and/or host cell entry, and the corresponding host protein with which the viral polypeptide interacts to recognize and/or enter a host cell. By way of example only, in some embodiments, a viral polypeptide receptor binding score is or comprises an ACE2 binding score. In some embodiments, an ACE2 binding score is a measure of the binding affinity between the S protein or a portion of the S protein (e.g., the RBD domain) and the ACE2 protein. In some embodiments, an ACE2 binding score can be generated using a conformational sampling algorithm. In some embodiments, an ACE2 binding score can be generated using structures that have been further optimized using a probabilistic optimization algorithm, a variant of simulated annealing, aiming to overcome local energy barriers and follow a kinetically accessible path toward an attainable deep energy minimum with respect to a knowledge-based, protein-oriented potential.

In some embodiments, a viral polypeptide binding score can be calculated using the change in the surface accessible surface area (SASA) between the bound and the unbound structures of a viral polypeptide and a host protein. In some embodiments, the SASA measurements can then be aggregated per variant (e.g., RBD variant) using medians. In some embodiments, each metric can be normalized by the metric relative to a reference sequence (e.g., wild type sequence or an RBD sequence having no mutations), such that the binding score for the reference sequence is one.

In some embodiments, a viral polypeptide binding score can be calculated using the change in Gibbs free energy between the bound and unbound states, e.g., using the change in binding energy when the interface forming chains are separated, versus when they are complexed. In some embodiments, the binding energy measurements can be aggregated per variant (e.g., RBD variant) using medians. In some embodiments, each metric can be normalized relative to a reference sequence (e.g., a wild type sequence, corresponding to no mutation on target domain (e.g., RBDs)) such that the binding score for the reference sequence is one.

In some embodiments, variant sequences having combinations of mutations, representing very rare viral polypeptide, for example, in some embodiments corresponding to less than 10% of all known sequence, can be excluded from such binding score analysis. Without wishing to be bound by a particular theory, such exclusion can be useful to improve computational efficiency. By way of example only, in some embodiments, sequences having other RBD mutation combinations, representing very rare RBDS, corresponding to <9% of all known sequences, can be excluded from such binding score analysis.

### Growth Score

In some embodiments, a growth score can be calculated using data provided in a publicly available database (e.g., GISAID metadata). In some embodiments, a growth score is calculated using recently submitted data. For example, in some embodiments, a growth score is calculated using data that have been submitted within the last 6 months (e.g., data that have been provided in the last 5 months, the last 4 months, the last 3 months, or the last two months, or within the last month). In some embodiments, a growth score is calculated using data that have been collected in the last eight weeks. In some embodiments, for each variant or lineage thereof, its growth can be calculated by determining its proportional change in a population of variants (e.g., among all submissions of sequences) over time (e.g., by comparing its proportion of the population at two points in time). By way of example only, in some embodiments, growth of a variant or lineage thereof can be calculated by the ratio of the proportion of the variant or lineage thereof determined over a recent time window (e.g., within last week), rₗₐₛₜ, to the proportion observed over a more extended time window (e.g., a time window that goes beyond the recent time window, e.g., an eight-week window), r_{win}. The ratio of rₗₐₛₜ/r_{win} is a measure of the change of the proportion. Ratio values larger than one indicate that the variant or the lineage thereof is rising and ratio values less than one indicate that the variant or lineage thereof is declining.

In some embodiments, an infectivity score (also known as a fitness prior score) described herein can reference a combination of a viral polypeptide receptor binding score and a log likelihood score. In some embodiments, an infectivity score (also known as a fitness prior score) described herein can reference a combination of a viral polypeptide receptor binding score, a log likelihood score, and a growth score. In some embodiments, experimental data (including, e.g., in vitro data) can be used to validate an infectivity score. For example, in some embodiments, binding affinity analysis between a target variant polypeptide and a cognate viral polypeptide receptor (e.g., RBD: ACE2 affinity analysis) can be performed to validate infectivity/transmissibility metric. Such affinity analysis can be performed using in vitro data that are already available and/or based on wet lab experiments using recombinant constructs of target polypeptides (e.g., RBD) from variants being assessed.

### Scores Scaling and Merging

In some embodiments, to make the semantic change, log-likelihood, epitope score, viral polypeptide receptor binding score (e.g., ACE2 binding score) and growth rate capable of being compared directly, a scaling strategy is introduced. For a given metric m, all the variants considered can be ranked according to this metric. In the ranking system used, the higher rank the better. In some embodiments, variants with the same value for metric m will get the same rank. In some embodiments, the ranks are then transformed into values between 0 and 100 through a linear projection to obtain the values for the scaled metric m_{scaled}. In some embodiments, all computed scores can be scaled as described herein. In some embodiments, all computed scores, except for log-likelihood, can be scaled, for example, in some embodiments where variants may have a large number of mutations, e.g., more than 30 mutations, more than 40 mutations, more than 50 mutations, more than 60 mutations, more than 70 mutations, or higher. In some embodiments, log-likelihood may penalize variants with a large number of mutations. Without wishing to be bound by any particular theory, an increased number of mutations may impact fitness, explaining the decreased log-likelihood. However, given that variants scored using methods and/or systems disclosed herein have been registered, this suggests that they have managed to infect hosts and replicate sufficiently to be detected, and that they have at least minimal fitness. By way of example only, a variant with two mutations, whose log-likelihood is in the bottom 20^{th} percentile globally, may be less likely to survive evolutionary competition, while a variant, with analogous log-likelihood, but with twenty mutations may be more likely to survive evolutionary competition as compared to similarly mutated variants. In some such embodiments, a conditional log-likelihood score is introduced such that the log-likelihood of variants having high mutational load is ranked relative to other variants with a similar mutation rate, as opposed to rank them across all variants. Thus, in some embodiments, a group-based ranking strategy can be used, where each variant is ranked among variants with a similar number of mutations (e.g., within 10% difference). For example, in some embodiments, for each variant, having N mutations, its log-likelihood score is ranked among all variants having at least M mutations, wherein M is the less of an arbitrary value (e.g., about 100, about 75, about 50, or about 25) and N minus 0 to 20 (e.g., N minus about 5 to 15, or N minus about 10). In some embodiments, M=min(max(0, N−10), 50). In some embodiments, N-terminal and C-terminal deletions are considered as a single mutation for grouping purposes. In some embodiments, for each group, the ranks are then transformed into values between 0 and 100 through a linear projection to obtain the values for the scaled metric. In some embodiments, results may be largely robust to the choice of a threshold.

In some embodiments, the immune escape score is computed as the average of the scaled semantic change and of the scaled epitope score. In some embodiments, the infectivity score is computed as the sum of the scaled log-likelihood, the scaled viral polypeptide receptor binding score (e.g., ACE2 binding score) and the scaled growth rate. In some embodiments, the infectivity score is computed as the sum of the scaled conditional log-likelihood, the scaled viral polypeptide receptor binding score (e.g., ACE2 binding score) and the scaled growth rate.

### Pareto Score

In some embodiments, an immune escape score (e.g., as described herein) and a fitness prior score (e.g., as described herein) can be combined to yield a Pareto score. In some embodiments, a Pareto score is based on Pareto optimality. In some embodiments, Pareto optimality is defined over a set of lineages. In some embodiments, lineages are Pareto optimal within a set if there are no lineages in the set with both higher immune escape and higher fitness prior scores. In some embodiments, a Pareto score is a measure of the degree of Pareto optimality. Lineages with the highest Pareto score are Pareto optimal. Lineages with the second-best Pareto score would be Pareto optimal, if the Pareto optimal lineages were removed from the set, and so on.

In some embodiments, a Pareto score can be determined by computing all the Pareto fronts that exist in a considered set of lineages. By way of example only, the first Pareto front corresponds to a set of lineages for which there does not exist any other lineage with both higher immune escape and fitness prior score. The second Pareto front is computed as the Pareto front over the set of lineages remaining when removing the ones from the first Pareto front. Successive Pareto fronts are computed until all the lineages are assigned to a front. In some embodiments, a linear projection can be used so that the lineages from the first front obtain a Pareto score of 100 and the ones from the last front get a Pareto score of 0.

In some embodiments, experimental data (including, e.g., in vitro data) can be used to validate whether variants computationally designated as elevated risk constitute real threat.

### Early Warning System (EWS) for Detecting Variants of Interest

In some embodiments, the disclosure provides an Early Warning System (EWS) for detecting one or more variants of interest, wherein the system comprises technologies for identifying a SARS-CoV-2 variant of interest using a method disclosed herein. In some embodiments, a variant of interest is a variant that has an increased likelihood of spreading in a population. In some embodiments, a variant of interest is a variant that has an increased likelihood of infecting more subjects in a population. In some embodiments, a variant of interest is a variant that has an increased likelihood of representing a greater portion of infected subjects in the near future. In some embodiments, an EWS described herein is useful for identifying variants that are considered as “High Risk Variants” (HRV). In some embodiments, an EWS described herein is useful for identifying variants that are considered as “Variants of concern” (VOC). In some embodiments, an EWS described herein is useful for identifying variants that are considered as “Variants of Interest” (VOI). In some embodiments, an EWS described herein is useful for identifying variants that are considered as “Variants under Monitoring” (VUM).

In some embodiments, the EWS comprises technologies for notifying relevant health agencies, monitoring agencies, and/or communities of the identified variant of interest. In some embodiments, the notification is performed within 2 months of identifying a variant of interest. In some embodiments, the notification is performed within 1 month, 3 weeks, 2 weeks, or 1 week of identifying a variant of interest.

In some embodiments, the EWS further comprises technologies for contact tracing of an identified variant of interest. In some embodiments, the EWS further comprises technologies for periodic sampling and/or environmental monitoring of the identified variant of interest. In some embodiments, the EWS further comprises technologies for reporting the identified variant of interest. In some embodiments, the EWS further comprise technologies for identifying a SARS-CoV-2 variant of interest within a period of time that is less than 1 month from first detecting a sequence (e.g., a period of time that is less than 3 weeks, less than 2 weeks, or less than 1 week after the first detection and reporting of a sequence of a variant of interest).

In some embodiments, the methods disclosed herein comprise assessing the risk of a variant polypeptide as compared to other variants. To jointly score the relative risks of variants using immune escape potential and infectivity, an optimality score, termed Pareto score, can be used to assess variants. The Pareto score is a mathematically robust way to identify lineages that are both immune escaping and infectious, and captures the relative evolutionary advantage of a given strain (see Examples for calculation details). For each lineage, as defined by the Pango nomenclature system (Rambaut et al., 2020), Pareto scores can be calculated by averaging the scores of the individual sequences belonging to a given lineage. A high Pareto score at a given time for a specific lineage indicates that only a few other lineages have higher scores for infectivity and immune escape at that time. As the Pareto score is a ranking system, and is calculated using values that are determined using machine learning algorithms that are frequently updated with new data, the Pareto score for a given variant can change over time, depending on what other variants are present in the subject population, and the data that the machine learning algorithms has been trained on.

In some embodiments, Pareto optimality is defined over a set of lineages. Lineages are Pareto optimal within that set if there are no lineages in the set with both higher immune escape and higher fitness prior scores. The Pareto score is a measure of the degree of Pareto optimality. Lineages with the highest Pareto score are Pareto optimal. Lineages with the second-best Pareto score would be Pareto optimal, if the Pareto optimal lineages were removed from the set, and so on.

In some embodiments, to compute the Pareto score, all the Pareto fronts that exist in the considered set of lineages are first calculated. The first Pareto front corresponds to the set of lineages for which there does not exist any other lineage with both higher immune escape and fitness prior score. The second Pareto front is computed as the Pareto front over the set of lineages remaining when removing the ones from the first Pareto front. Successive Pareto fronts can be computed until all lineages are assigned to a front. In some embodiments, a linear projection can be used so that the lineages from the first front obtain a Pareto score of 100 and the ones from the last front get a Pareto score of 0.

### Detection

In some embodiments, during retrospective early detection analysis, at each occasion the EWS run, the system considers only the new sequences reported since the last time the EWS was run (e.g., in some embodiments the EWS is run on a weekly basis, and is only used to evaluate the new variants that have been detected in the past week). Thus, in some embodiments, each sequence is considered only once at the time of its first report. Furthermore, in some embodiments, to prevent consistently detecting sequences of prevalent lineages (such as the Alpha variant of SARS-CoV-2), EWS does not consider sequences of the Variants of Concern that were designated as such at the time of evaluation.

In some embodiments, the immune escape score alone (e.g., without the infectivity score) may be used to detect variants of concern. One advantage of the immune escape score is that it relies on sequence alone, and unlike the described infectivity score does not require growth metrics, which are not available when a novel variant gets sequenced. Accordingly, one advantage of an early warning system that does not use an infectivity score is that it can be capable of spotting dangerous variants at an earlier point in time.

In some embodiments, the detection systems disclosed here are capable of identifying a variant as a variant of elevated risk 20 days or more earlier than traditional variant detection systems (e.g., systems that depend solely on growth rate). In some embodiments, the detection systems disclosed here are capable of identifying a variant as a variant of elevated risk 30 days or more earlier than traditional variant detection systems (e.g., systems that depend solely on growth rate). In some embodiments, the detection systems disclosed here are capable of identifying a variant as a variant of elevated risk 40 days or more earlier than traditional variant detection systems (e.g., systems that depend solely on growth rate). In some embodiments, the detection systems disclosed here are capable of identifying a variant as a variant of elevated risk 50 days or more earlier than traditional variant detection systems (e.g., systems that depend solely on growth rate). In some embodiments, the detection systems disclosed here are capable of identifying a variant as a variant of elevated risk 72 days or more earlier than traditional variant detection systems (e.g., systems that depend solely on growth rate).

In some embodiments, the detection systems disclosed herein are capable of identifying a variant as a variant of elevated risk after detection of less than 1,000 sequences. In some embodiments, the detection systems disclosed herein are capable of identifying a variant as a variant of elevated risk after detection of less than 500 sequences. In some embodiments, the detection systems disclosed herein are capable of identifying a variant as a variant of elevated risk after detection of less than 200 sequences. In some embodiments, the detection systems disclosed herein are capable of identifying a variant as a variant of elevated risk after detection of less than 100 sequences. In some embodiments, the detection systems disclosed herein are capable of identifying a variant as a variant of elevated risk after detection of less than 50 sequences.

In some embodiments, technologies disclosed herein (including, e.g., methods and systems disclosed herein) incorporate one or more (e.g., 1, 2, 3, 4, or 5) of scores described herein, which can be selected from: epitope alteration score, semantic change score, viral polypeptide receptor binding score, log-likelihood score, and growth score. In some embodiments, technologies disclosed herein (including, e.g., methods and systems disclosed herein) incorporate one or more (e.g., 1, 2, 3, 4, or 5) of scores summarized in Table 1, below. Such 5 scores can be grouped into immune escape and fitness prior scores as described herein. In some embodiments, each of such 5 scores can be normalized so as to give a value 0 and 100%. In some embodiments, the average of such-scores in each score category can be used to compute immune escape and fitness prior scores as described herein.

TABLE 1
Certain embodiments of scores that can be utilized in exemplary EWS described herein

| Score Name | Score Category | Exemplary Embodiments |
|---|---|---|
| Epitope Alteration | Immune Escape | Measures the alteration of a variant polypeptide |
| Score (also known | Score | (e.g., Spike polypeptide) at epitope positions (e.g., |
| as Epitope Score) | | positions that have been previously shown to |
| | | interact with neutralizing antibodies). In some |
| | | embodiments, such a measure can be determined |
| | | by counting the number of antibodies potentially |
| | | escaped. |
| Semantic Change | Immune Escape | Measures the functional change of a variant |
| Score | Score | polypeptide (e.g., a Spike polypeptide) using, e.g., |
| | | transformer-derived embedding differences with |
| | | respect to one or more reference sequences (e.g., to |
| | | wild type and known variant Spike polypeptide |
| | | sequences). |
| Viral Polypeptide | Fitness Prior Score | Approximates binding affinity using binding |
| Receptor Binding | (also known as | energies estimated from in silico simulations, |
| Score | Infectivity Score) | which in some embodiments can be characterized |
| | | by changes in Gibbs free energy and/or solvent |
| | | accessible surface areas estimated from in-silico |
| | | simulations. |
| Log-Likelihood | Fitness Prior Score | Log-likelihood score - Measures the existence |
| Score or | (also known as | probability of a variant polypeptide sequence (e.g., |
| Conditional Log- | Infectivity Score) | a Spike polypeptide sequence), for example, using |
| likelihood Score | | Transformer-derived log-likelihoods without |
| | | reference. |
| | | Conditional log-likelihood score - Measures the |
| | | relative rank of the measured existence probability |
| | | of a variant polypeptide sequence (e.g., a Spike |
| | | polypeptide sequence), for example, using |
| | | Transformer-derived log-likelihoods with reference |
| | | to other sequences with similar mutation count. |
| Growth Score | Fitness Prior Score | Calculates growth (e.g., lineage-level growth), for |
| | (also known as | example, using deposited sequence metadata |
| | Infectivity Score) | (retrospective data). |

### RNA Polynucleotides

In certain embodiments of the present disclosure, the RNA encoding one or more variants of interest as determined or characterized by methods described herein is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

RNA polynucleotides (e.g., RNA polynucleotides encoding S protein from SARS-CoV-2 variants), LNP or lipoplex formulations comprising the same, vaccine formulations comprising the same, and methods for manufacturing each of the same are known in the art. See, e.g., WO2021214204; WO2021213924A1, WO2021213945A1, US 20210228707, WO/2021/159130, WO 2021/154763, WO2021159040A2, and WO2021/222304, each of which is incorporated herein by reference in its entirety for purposes described herein.

In some embodiments, an RNA molecule described herein comprises at least one non-coding sequence element. In some embodiments, such a non-coding sequence element is included in an RNA molecule to enhance RNA stability and/or translation efficiency. Examples of non-coding sequence elements include but are not limited to a 3′ untranslated region (UTR), a 5′ UTR, a cap structure, a poly adenine (polyA) tail, and any combinations thereof.

UTRs (5′ UTRs and/or 3′UTRs)

In some embodiments, a provided RNA molecule comprises a nucleotide sequence that encodes a 5′UTR of interest and/or a 3′ UTR of interest. One of skill in the art will appreciate that untranslated regions (e.g., 3′ UTR and/or 5′ UTR) of an mRNA sequence can contribute to mRNA stability, mRNA localization, and/or translational efficiency.

In some embodiments, a provided RNA molecule can comprise a 5′ UTR nucleotide sequence and/or a 3′ UTR nucleotide sequence. In some embodiments, such a 5′ UTR sequence can be operably linked to a 3′ of a coding sequence (e.g., encompassing one or more coding regions). Additionally or alternatively, in some embodiments, a 3′ UTR sequence can be operably linked to 5′ of a coding sequence (e.g., encompassing one or more coding regions).

In some embodiments, 5′ and 3′ UTR sequences included in an RNA molecule described herein can consist of or comprise naturally occurring or endogenous 5′ and 3′ UTR sequences for an open reading frame of a gene of interest. Alternatively, in some embodiments, 5′ and/or 3′ UTR sequences included in an RNA molecule are not endogenous to a coding sequence (e.g., encompassing one or more coding regions); in some such embodiments, such 5′ and/or 3′ UTR sequences can be useful for modifying the stability and/or translation efficiency of an RNA sequence transcribed. For example, a skilled artisan will appreciate that AU-rich elements in 3′ UTR sequences can decrease the stability of mRNA. Therefore, as will be understood by a skilled artisan, 3′ and/or 5′ UTRs can be selected or designed to increase the stability of the transcribed RNA based on properties of UTRs that are well known in the art.

For example, one skilled in the art will appreciate that, in some embodiments, a nucleotide sequence consisting of or comprising a Kozak sequence of an open reading frame sequence of a gene or nucleotide sequence of interest can be selected and used as a nucleotide sequence encoding a 5′ UTR. As will be understood by a skilled artisan, Kozak sequences are known to increase the efficiency of translation of some RNA transcripts, but are not necessarily required for all RNAs to enable efficient translation. In some embodiments, a provided RNA molecule can comprise a nucleotide sequence that encodes a 5′ UTR derived from an RNA virus whose RNA genome is stable in cells. In some embodiments, various modified ribonucleotides (e.g., as described herein) can be used in the 3′ and/or 5′ UTRs, for example, to impede exonuclease degradation of the transcribed RNA sequence.

In some embodiments, a 5′ UTR included in an RNA molecule described herein may be derived from human α-globin mRNA combined with Kozak region.

In some embodiments, an RNA molecule may comprise one or more 3′UTRs. For example, in some embodiments, an RNA molecule may comprise two copies of 3′-UTRs derived from a globin mRNA, such as, e.g., alpha2-globin, alpha1-globin, beta-globin (e.g., a human beta-globin) mRNA. In some embodiments, two copies of 3′UTR derived from a human beta-globin mRNA may be used, e.g., in some embodiments which may be placed between a coding sequence of an RNA molecule and a poly(A)-tail, to improve protein expression levels and/or prolonged persistence of an mRNA. In some embodiments, a 3′UTR derived from a human beta-globin as described in WO 2007/036366, the contents of which are incorporated herein by reference in their entireties for the purposes described herein, may be included in an RNA molecule described herein.

In some embodiments, a 3′ UTR included in an RNA molecule may be or comprise one or more (e.g., 1, 2, 3, or more) of the 3′UTR sequences disclosed in WO 2017/060314, the entire content of which is incorporated herein by reference for the purposes described herein. In some embodiments, a 3′-UTR may be a combination of at least two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see WO 2017/060314, herein incorporated by reference).

In some embodiments, a 3′-UTR sequence comprises a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These sequences were identified by an ex vivo selection process for sequences that confer RNA stability and augment total protein expression (see WO 2017/060314, herein incorporated by reference). Alternatively, the 3′-UTR may be two re-iterated 3′-UTRs of the human beta-globin mRNA.

### PolyA Tail

In some embodiments, a provided RNA can comprise a nucleotide sequence that encodes a polyA tail. A polyA tail is a nucleotide sequence comprising a series of adenosine nucleotides, which can vary in length (e.g., at least 5 adenine nucleotides) and can be up to several hundred adenosine nucleotides. In some embodiments, a polyA tail is a nucleotide sequence comprising at least 30 adenosine nucleotides or more, including, e.g., at least 35, at least 40, at least 45, at least 50, at least 55, at least 60, at least 65, at least 70, at least 75, at least 80, at least 85, at least 90, at least 95, at least 100, or more adenosine nucleotides. In some embodiments, a polyA tail is a nucleotide sequence comprising at least 120 adenosine nucleotides. In some embodiments, a polyA tail as described in WO 2007/036366, the contents of which are incorporated herein by reference in their entireties for the purposes described herein, may be included in an RNA molecule described herein.

In some embodiments, a polyA tail is or comprises a polyA homopolymeric tail. In some embodiments, a polyA tail may comprise one or more modified adenosine nucleosides, including, but not limited to, cordiocipin and 8-azaadenosine.

In some embodiments, a polyA tail may comprise one or more non-adensoine nucleotides. In some embodiments, a polyA tail may be or comprise a disrupted or modified polyA tail as described in WO 2016/005324, the entire content of which is incorporated herein by reference for the purpose described herein. For example, in some embodiments, a polyA tail included in an RNA molecule described herein may be or comprise a modified polyA sequence comprising: a linker sequence; a first sequence of at least 20 A consecutive nucleotides, which is 5′ of the linker sequence; and a second sequence of at least 20 A consecutive nucleotides, which is 3′ of the linker sequence. In some embodiments, a modified polyA sequence may comprise: a linker sequence comprising at least ten non-A nucleotides (e.g., T, G, and/or C nucleotides); a first sequence of at least 30 A consecutive nucleotides, which is 5′ of the linker sequence; and a second sequence of at least 70 A consecutive nucleotides, which is 3′ of the linker sequence.

### 5′ Cap

In some embodiments, an RNA molecule described herein may comprise a 5′ cap, which may be incorporated into such an RNA molecule during transcription, or joined to such an RNA molecule post-transcription. In some embodiments, an RNA molecule may comprise an anti-reverse cap analog (ARCA). In some embodiments, an RNA molecule may comprise a cap analog beta-S-ARCA(D1) (m₂^{7,2′-O}GppₛpG) as illustrated below:

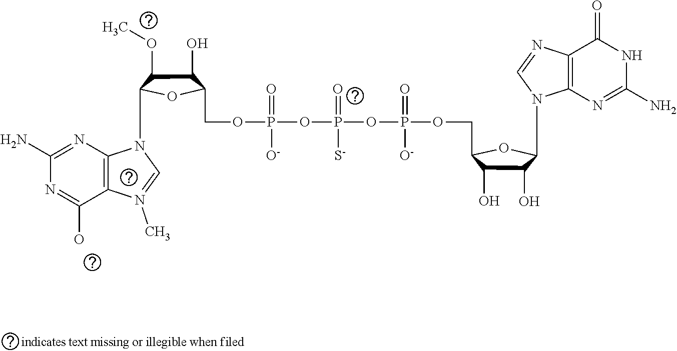

In some embodiments, an RNA molecule may comprise an S-ARCA cap structure as disclosed in WO2011/015347 or in WO2008/157688, the entire contents of each of which are incorporated herein by reference for the purposes described herein.

In some embodiments, an RNA molecule may comprise a 5′ cap structure for co-transcriptional capping of mRNA. Examples of a cap structure for co-transcriptional capping are known in the art, including, e.g., as described in WO 2017/053297, the entire content of which is incorporated herein by reference for the purposes described herein. In some embodiments, a 5′ cap included in an RNA molecule described herein is or comprises m7G(5′)ppp(5′)(2′OMeA)pG. In some embodiments, a 5′ cap included in an RNA molecule described herein is or comprises a Cap1 structure [e.g., but not limited to m7(3′OMeG)(5′)ppp(5′)(2′OMeA)pG].

In some embodiments, the RNA polynucleotides disclosed herein comprise natural ribonucleotides. In some embodiments, the RNA polynucleotides disclosed herein comprise at least one modified or synthetic ribonucleotide. In some embodiments, modified or synthetic ribonucleotides are included in an RNA molecule to increase its stability and/or to decrease its cytotoxicity. For example, in some embodiments, at least one of A, U, C, and G ribonucleotide of an RNA molecule described herein may be replaced by a modified ribonucleotide. For example, in some embodiments, some or all of cytidine residues present in an RNA molecule may be replaced by a modified cytidine, which in some embodiments may be, e.g., 5-methylcytidine. Alternatively or additionally, in some embodiments, some or all of uridine residues present in an RNA molecule may be replaced by a modified uridine, which in some embodiments may be, e.g., pseudoridine, such as, e.g., 1-methylpseudouridine. In some embodiments, all uridine residues present in an RNA molecule is replaced by pseudouridine, e.g., 1-methylpseudouridine.

In some embodiments, the present disclosure, among other things, provides a pharmaceutical composition including one or more RNA molecules where an RNA molecule comprises from 5′ to 3′: (i) a 5′ cap or 5′ cap analogue; (ii) at least one 5′ UTR; (iii) a signal peptide; (iv) a coding region that encodes at least one antigen derived from a viral variant that has been identified using a method disclosed herein; (v) at least one 3′UTR; and (vi) a poly-adenine tail. For example, in some embodiments, a cap structure that is included in an RNA molecule described herein can be a cap structure that can increase the resistance of RNA molecules to degradation by extracellular and intracellular RNases and leads to higher protein expression. In some embodiments, an exemplary cap structure is or comprises beta-S-ARCA(D1) (m₂^{7,2′-O}GppₛpG). In some embodiments, an exemplary cap structure is or comprises m7(3′OMeG)(5′)ppp(5′)(2′OMeA)pG. In some embodiments, an exemplary 5′ UTR sequence element that is included in an RNA molecule described herein is or comprises a characteristic sequence from human α-globin and a Kozak consensus sequence. In some embodiments, an exemplary 3′ UTR sequence element that is included in an RNA molecule described herein may be or comprise two copies of 3′UTR derived from a human beta-globin, or a combination of two sequence elements (FI element) derived from the “amino terminal enhancer of split” (AES) mRNA (called F) and a mitochondrial encoded 12S ribosomal RNA (called I). See, e.g., WO2007/036366 and WO 2017/060314, the entire contents of each of which is incorporated herein by reference for the purposes described herein. In some embodiments, a poly(A)-tail that is included in an RNA molecule described herein can be designed to enhance RNA stability and/or translational efficiency. In some embodiments, an exemplary poly(A)-tail is or comprises a contiguous poly(A) sequence of at least 120 adenosine nucleotides in length. In some embodiments, an exemplary poly(A)-tail is or comprises a modified poly(A) sequence of 110 nucleotides in length including a stretch of 30 adenosine residues, followed by a 10 nucleotide linker sequence and another stretch of 70 adenosine residues (A30L70).

In one embodiment, RNA is in vitro transcribed RNA (IVT-RNA) and may be obtained by in vitro transcription of an appropriate DNA template.

In one embodiment, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

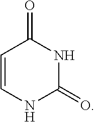

The term “uridine,” as used herein, describes one of the nucleosides that can occur in RNA. The structure of uridine is:

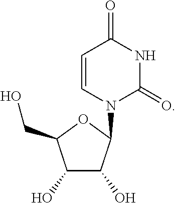 [SMILES: O=c1ccn([C@@H]2O[C@H](CO)[C@@H](O)[C@H]2O)c(=O)[nH]1]

UTP (uridine 5′-triphosphate) has the following structure:

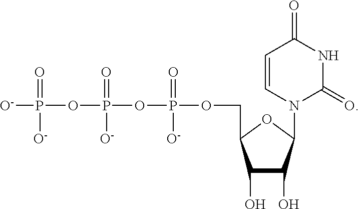

Pseudo-UTP (pseudouridine 5′-triphosphate) has the following structure:

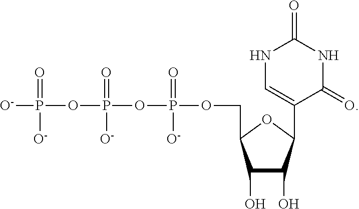

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1Ψ), which has the structure:

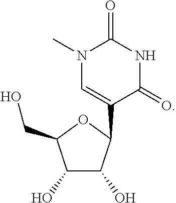 [SMILES: Cn1cc([C@@H]2O[C@H](CO)[C@@H](O)[C@H]2O)c(=O)[nH]c1=O]

N1-methyl-pseudo-UTP has the following structure:

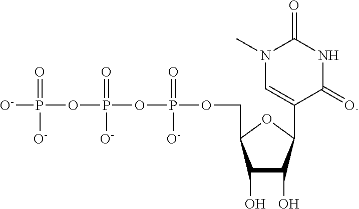

Another exemplary modified nucleoside is 5-methyl-uridine (m5U), which has the structure:

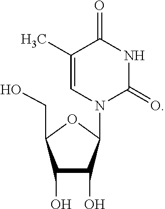

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the RNA polynucleotide encodes the Wuhan strain of SARS-CoV-2 and one or more mutations that have been determined to elevate the risk of a variant polypeptide, using any one of the methods disclosed herein. In some embodiments, the Spike sequences identified herein may be modified in such a way that the prototypical prefusion conformation is stabilized. Stabilization of the prefusion conformation may be obtained by introducing two consecutive proline substitutions at AS residues 986 and 987 in the full length spike protein. Specifically, spike (S) protein stabilized protein variants are obtained in a way that the amino acid residue at position 986 is exchanged to proline and the amino acid residue at position 987 is also exchanged to proline, e.g., as shown in SEQ ID NO: 7, below, which comprises the proline mutations at residues 986 and 987 in the Spike protein from the Wuhan strain.

In some embodiments, the RNA polynucleotides are single-stranded RNA that may be translated into the respective protein upon entering cells of a recipient. In addition to wild-type (e.g., the native nucleic acid of the variant) or codon-optimized sequences encoding the antigen sequence, the RNA may contain one or more structural elements optimized for maximal efficacy of the RNA with respect to stability and translational efficiency (e.g., a 5′ cap, 5′ UTR, 3′ UTR, poly(A)-tail). In one embodiment, the RNA contains all of these elements. In one embodiment, beta-S-ARCA(D1) (m27,2′-OGppSpG) or m27,3′-OGppp(m12′-O)ApG may be utilized as specific capping structure at the 5′-end of the RNA drug substances. As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These were identified by an ex vivo selection process for sequences that confer RNA stability and augment total protein expression (see WO 2017/060314, herein incorporated by reference). Alternatively, the 3′-UTR may be two re-iterated 3′-UTRs of the human beta-globin mRNA. Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency.

Furthermore, a secretory signal peptide (sec) may be fused to the antigen-encoding regions preferably in a way that the sec is translated as N terminal tag. In one embodiment, sec corresponds to the secretory signal peptide of the S protein. Sequences coding for short linker peptides predominantly consisting of the amino acids glycine (G) and serine (S), as commonly used for fusion proteins may be used as GS/Linkers.

The vaccine RNA described herein may be complexed with proteins and/or lipids, preferably lipids, to generate RNA-particles for administration. If a combination of different RNAs is used, the RNAs may be complexed together or complexed separately with proteins and/or lipids to generate RNA-particles for administration.

In one aspect, the invention relates to a composition or medical preparation comprising RNA encoding an amino acid sequence comprising a SARS-CoV-2 S protein, an immunogenic variant thereof, or an immunogenic fragment of the SARS-CoV-2 S protein or the immunogenic variant thereof.

In one embodiment, an immunogenic fragment of the SARS-CoV-2 S protein comprises the S1 subunit of the SARS-CoV-2 S protein, or the receptor binding domain (RBD) of the S1 subunit of the SARS-CoV-2 S protein.

In one embodiment, the amino acid sequence comprising a SARS-CoV-2 S protein, an immunogenic variant thereof, or an immunogenic fragment of the SARS-CoV-2 S protein or the immunogenic variant thereof is able to form a multimeric complex, in particular a trimeric complex. To this end, the amino acid sequence comprising a SARS-CoV-2 S protein, an immunogenic variant thereof, or an immunogenic fragment of the SARS-CoV-2 S protein or the immunogenic variant thereof may comprise a domain allowing the formation of a multimeric complex, in particular a trimeric complex of the amino acid sequence comprising a SARS-CoV-2 S protein, an immunogenic variant thereof, or an immunogenic fragment of the SARS-CoV-2 S protein or the immunogenic variant thereof. In one embodiment, the domain allowing the formation of a multimeric complex comprises a trimerization domain, for example, a trimerization domain as described herein.

In one embodiment, the amino acid sequence comprising a SARS-CoV-2 S protein, an immunogenic variant thereof, or an immunogenic fragment of the SARS-CoV-2 S protein or the immunogenic variant thereof is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, RNA described herein can comprise one or more of the sequences shown in Table 2, or functional portions thereof.

TABLE 2
DESCRIPTION OF THE SEQUENCES

| | | |
|---|---|---|
| SEQ | | |
| ID NO: | Description | SEQUENCE |

Antigenic S protein sequences

| | | |
|---|---|---|
| 1 | S protein | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEK |
| | (amino acid) | SNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFV |
| | | FKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCAL |
| | | DPLSETKCTLKSFTVEKGIYQTSNFRVQPTESIVRFPNITNLCPFGEVFNATRFASVYAWNRKRISNCVADYSVLYNSASFSTFKCYGVSPTKLNDLCF |
| | | TNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEIL |
| | | DITPCSFGGVSVITPGTNTSNQVAVLYQDVNCTEVPVAIHADQLTPTWRVYSTGSNVFQTRAGCLIGAEHVNNSYECDIPIGAGICASYQTQTNSP |
| | | RRARSVASQSIIAYTMSLGAENSVAYSNNSIAIPTNFTISVTTEILPVSMTKTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAG |
| | | TITSGWTFGAGAALQIPFAMQMAYRFNGIGVTQNVLYENQKLIANQFNSAIGKIQDSLSSTASALGKLQDVVNQNAQALNTLVKQLSSNFGAISS |
| | | VLNDILSRLDKVEAEVQIDRLITGRLQSLQTYVTQQLIRAAEIRASANLAATKMSECVLGQSKRVDFCGKGYHLMSFPQSAPHGVVFLHVTYVPAQE |
| | | KNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDI |
| | | SGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKG |
| | | VKLHYT |
| 2 | S protein | auguuuguguuucuugugcugcugccucuugugucuucucagugugugaauuugacaacaagaacacagcugccaccagcuuauacaaauucuuuuaccagaggagug |
| | (CDS) | uauuauccugauaaaguguuuagaucuucugugcugcacagcacacaggaccuguuucugccauuuuuuagcaaugugacaugguuucaugcaauucaugugucugg |
| | | aacaaauggaacaaaaagauuugauaauccugugcugccuuuuaaugauggaguguauuuugcuucaacagaaaagucaaauauuauuagaggauggauuuuugga |
| | | acaacacuggauucuaaaacacagucucugcugauugugaauaaugcaacaaauguggugauuaaagugugugaauuucaguuuuguaaugauccuuuucugggagu |
| | | guauuaucacaaaaauaauaaaucuuggauggaaucugaauuuagaguguauuccucugcaaauaauuguacauuugaauaugugucucagccuuuucugauggau |
| | | cuggaaggaaaacagggcaauuuuaaaaaucugagagaauuuguguuuaaaaauauugauggauauuuuaaaauuuauucuaaacacacaccaauuaauuuaguga |
| | | gagaucugccucagggauuuucugcucuggaaccucugguggaucugccaauuggcauuaauauuacaagauuucagacacugcuggcucugcacagaucuuaucugac |
| | | accuggagauucuucuucuggauggacagccggagcugcagcuuauuaugugggcuaucugcagccaagaacauuucugcugaaauauaaugaaaauggaacaauuac |
| | | agaugcuguggauugugcucuggauccucugucugaaacaaaauguacauuaaaaucuuuuacaguggaaaaaggcauuuaucagacaucuaauuuuagagugcagc |
| | | caacagaaucuauugugagauuuccaaauauuacaaaucuguguccauuuggagaaguguuuaaugcaacaagauuugcaucuguguaugcauggaauagaaaaaga |
| | | auuucuaauuguguggcugauuauucugugcuguauaauagugcuucuuuuuccacauuuaaauguuauggagugucuccaacaaaauuaaaugauuuauguuuu |
| | | acaaauguguaugcugauucuuuugugaucagaggugaugaagugagacagauugcccccggacagacaggaaaaauugcugauuacaauuacaaacugccugaugau |
| | | uuuacaggaugugugauugcuuggaauucuaauaauuuagauucuaaagugggaggaaauuacaauuaucuguacagacuguuuagaaaaucaaaucugaaaccuu |
| | | uugaaagagauauuucaacagaaauuuaucaggcuggaucaacaccuuguaauggaguggaaggauuuaauuguuauuuuccauuacagagcuauggauuucagcca |
| | | accaauggugugggauaucagccauauagagugguggugcugucuuuugaacugcugcaugcaccugcaacaguguguggaccuaaaaaaucuacaaauuuagugaaa |
| | | aauaaaugugugaauuuuaauuuuaauggauuaacaggaacaggagugcugacagaaucuaauaaaaaauuucugccuuuucagcaguuuggcagagauauugcag |
| | | auaccacagaugcagugagagauccucagacauuagaaauucuggauauuacaccuuguucuuuugggggugugucugugauuacaccuggaacaaauacaucuaauc |
| | | agguggcugugcuguaucaggaugugaauuguacagaagugccaguggcaauucaugcagaucagcugacaccaacauggagaguguauucuacaggaucuaaugugu |
| | | uucagacaagagcaggaugucugauuggagcagaacaugugaauaauucuuaugaaugugauauuccaauuggagcaggcauuugugcaucuuaucagacacagacaa |
| | | auuccccaaggagagcaagaucuguggcaucucagucuauuauugcauacaccaugucucugggagcagaaaauucuguggcauauucuaauaauucuauugcuauuc |
| | | caacaaauuuuaccauuucugugacaacagaaauuuuaccugugucuaugacaaaaacaucuguggauuguaccauguacauuuguggagauucuacagaauguucu |
| | | aaucugcugcugcaguauggaucuuuuuguacacagcugaauagagcuuuaacaggaauugcuguggaacaggauaaaaauacacaggaaguguuugcucaggugaaa |
| | | cagauuuacaaaacaccaccaauuaaagauuuuggaggauuuaauuuuagccagauucugccugauccuucuaaaccuucuaaaagaucuuuuauugaagaucugcu |
| | | guuuaauaaagugacacuggcagaugcaggauuuauuaaacaguauggagauugccugggugauauugcugcaagagaucugauuugugcucagaaauuuaauggac |
| | | ugacagugcugccuccucugcugacagaugaaaugauugcucaguacacaucugcuuuacuggcuggaacaauuacaagcggauggacauuuggagcuggagcugcucu |
| | | gcagauuccuuuugcaaugcagauggcuuacagauuuaauggaauuggagugacacagaauguguuauaugaaaaucagaaacugauugcaaaucaguuuaauucug |
| | | caauuggcaaaauucaggauucucugucuucuacagcuucugcucugggaaaacugcaggauguggugaaucagaaugcacaggcacugaauacucuggugaaacagcu |
| | | gucuagcaauuuuggggcaauuucuucugugcugaaugauauucugucuagacuggauaaaguggaagcugaagugcagauugauagacugaucacaggaagacugca |
| | | gucucugcagacuuaugugacacagcagcugauuagagcugcugaaauuagagcuucugcuaaucuggcugcuacaaaaaugucugaaugugugcugggacagucaaa |
| | | aagaguggauuuuuguggaaaaggauaucaucugaugucuuuuccacagucugcuccacauggagugguguuuuuacaugugacauaugugccagcacaggaaaagaa |
| | | uuuuaccacagcaccagcaauuugucaugauggaaaagcacauuuuccaagagaaggaguguuugugucuaauggaacacauugguuugugacacagagaaauuuuu |
| | | augaaccucagauuauuacaacagauaauacauuugugucaggaaauugugauguggugauuggaauugugaauaauacaguguaugauccacugcagccagaacug |
| | | gauucuuuuaaagaagaacuggauaaauauuuuaaaaaucacacaucuccugauguggauuuaggagauauuucuggaaucaaugcaucuguggugaauauucaga |
| | | aagaaauugauagacugaaugaaguggccaaaaaucugaaugaaucucugauugaucugcaggaacuuggaaaauaugaacaguacauuaaauggccuugguacauu |
| | | uggcuuggauuuauugcaggauuaauugcaauugugauggugacaauuauguuauguuguaugacaucauguuguucuuguuuaaaaggauguuguucuugugga |
| | | agcuguuguaaauuugaugaagaugauucugaaccuguguuaaaaggagugaaauugcauuacaca |
| 3 | S protein RBD | MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVR |
| | (amino acid) | QIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVG |
| | | YQPYRVVVLSFELLHAPATVCGPK |
| 4 | S protein RBD | auguuuguguuucuugugcugcugccucuugugucuucucaguguguggugagauuuccaaauauuacaaaucuguguccauuuggagaaguguuuaaugcaacaag |
| | (CDS) | auuugcaucuguguaugcauggaauagaaaaagaauuucuaauuguguggcugauuauucugugcuguauaauagugcuucuuuuuccacauuuaaauguuaugga |
| | | gugucuccaacaaaauuaaaugauuuauguuuuacaaauguguaugcugauucuuuugugaucagaggugaugaagugagacagauugcccccggacagacaggaaa |
| | | aauugcugauuacaauuacaaacugccugaugauuuuacaggaugugugauugcuuggaauucuaauaauuuagauucuaaagugggaggaaauuacaauuaucug |
| | | uacagacuguuuagaaaaucaaaucugaaaccuuuugaaagagauauuucaacagaaauuuaucaggcuggaucaacaccuuguaauggaguggaaggauuuaauug |
| | | uuauuuuccauuacagagcuauggauuucagccaaccaauggugugggauaucagccauauagagugguggugcugucuuuugaacugcugcaugcaccugcaacagu |
| | | guguggaccuaaa |
| 5 | S protein RBD/ | MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVR |
| | Fibritin | QIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVG |
| | (amino acid) | YQPYRVVVLSFELLHAPATVCGPKGSPGSGSGSGYIPEAPRDGQAYVRKDGEWVLLSTFLGRSLEVLFQGPG |
| 6 | S protein RBD/ | auguuuguguuucuugugcugcugccucuugugucuucucaguguguggugagauuuccaaauauuacaaaucuguguccauuuggagaaguguuuaaugcaacaag |
| | Fibritin (CDS) | auuugcaucuguguaugcauggaauagaaaaagaauuucuaauuguguggcugauuauucugugcuguauaauagugcuucuuuuuccacauuuaaauguuaugga |
| | | gugucuccaacaaaauuaaaugauuuauguuuuacaaauguguaugcugauucuuuugugaucagaggugaugaagugagacagauugcccccggacagacaggaaa |
| | | aauugcugauuacaauuacaaacugccugaugauuuuacaggaugugugauugcuuggaauucuaauaauuuagauucuaaagugggaggaaauuacaauuaucug |
| | | uacagacuguuuagaaaaucaaaucugaaaccuuuugaaagagauauuucaacagaaauuuaucaggcuggaucaacaccuuguaauggaguggaaggauuuaauug |
| | | uuauuuuccauuacagagcuauggauuucagccaaccaauggugugggauaucagccauauagagugguggugcugucuuuugaacugcugcaugcaccugcaacagu |
| | | guguggaccuaaaggcucccccggcuccggcuccggaucugguuauauuccugaagcuccaagagaugggcaagcuuacguucguaaagauggcgaauggguauuacuu |
| | | ucuaccuuuuuaggccggucccuggaggugcuguuccagggccccggc |
| 7 | S protein PP | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEK |
| | (amino acid) | SNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFV |
| | | FKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCAL |
| | | DPLSETKCTLKSFTVEKGIYQTSNFRVQPTESIVRFPNITNLCPFGEVFNATRFASVYAWNRKRISNCVADYSVLYNSASFSTFKCYGVSPTKLNDLCF |
| | | TNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNC |
| | | YFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEIL |
| | | DITPCSFGGVSVITPGTNTSNQVAVLYQDVNCTEVPVAIHADQLTPTWRVYSTGSNVFQTRAGCLIGAEHVNNSYECDIPIGAGICASYQTQTNSP |
| | | RRARSVASQSIIAYTMSLGAENSVAYSNNSIAIPTNFTISVTTEILPVSMTKTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAG |
| | | TITSGWTFGAGAALQIPFAMQMAYRFNGIGVTQNVLYENQKLIANQFNSAIGKIQDSLSSTASALGKLQDVVNQNAQALNTLVKQLSSNFGAISS |
| | | VLNDILSRLDPPEAEVQIDRLITGRLQSLQTYVTQQLIRAAEIRASANLAATKMSECVLGQSKRVDFCGKGYHLMSFPQSAPHGVVFLHVTYVPAQE |
| | | KNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDI |
| | | SGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKG |
| | | VKLHYT |
| 8 | S protein PP | auguuuguguuucuugugcugcugccucuugugucuucucagugugugaauuugacaacaagaacacagcugccaccagcuuauacaaauucuuuuaccagaggagug |
| | (CDS)(V08) | uauuauccugauaaaguguuuagaucuucugugcugcacagcacacaggaccuguuucugccauuuuuuagcaaugugacaugguuucaugcaauucaugugucugg |
| | | aacaaauggaacaaaaagauuugauaauccugugcugccuuuuaaugauggaguguauuuugcuucaacagaaaagucaaauauuauuagaggauggauuuuugga |
| | | acaacacuggauucuaaaacacagucucugcugauugugaauaaugcaacaaauguggugauuaaagugugugaauuucaguuuuguaaugauccuuuucugggagu |
| | | guauuaucacaaaaauaauaaaucuuggauggaaucugaauuuagaguguauuccucugcaaauaauuguacauuugaauaugugucucagccuuuucugauggau |
| | | cuggaaggaaaacagggcaauuuuaaaaaucugagagaauuuguguuuaaaaauauugauggauauuuuaaaauuuauucuaaacacacaccaauuaauuuaguga |
| | | gagaucugccucagggauuuucugcucuggaaccucugguggaucugccaauuggcauuaauauuacaagauuucagacacugcuggcucugcacagaucuuaucugac |
| | | accuggagauucuucuucuggauggacagccggagcugcagcuuauuaugugggcuaucugcagccaagaacauuucugcugaaauauaaugaaaauggaacaauuac |
| | | agaugcuguggauugugcucuggauccucugucugaaacaaaauguacauuaaaaucuuuuacaguggaaaaaggcauuuaucagacaucuaauuuuagagugcagc |
| | | caacagaaucuauugugagauuuccaaauauuacaaaucuguguccauuuggagaaguguuuaaugcaacaagauuugcaucuguguaugcauggaauagaaaaaga |
| | | auuucuaauuguguggcugauuauucugugcuguauaauagugcuucuuuuuccacauuuaaauguuauggagugucuccaacaaaauuaaaugauuuauguuuu |
| | | acaaauguguaugcugauucuuuugugaucagaggugaugaagugagacagauugcccccggacagacaggaaaaauugcugauuacaauuacaaacugccugaugau |
| | | uuuacaggaugugugauugcuuggaauucuaauaauuuagauucuaaagugggaggaaauuacaauuaucuguacagacuguuuagaaaaucaaaucugaaaccuu |
| | | uugaaagagauauuucaacagaaauuuaucaggcuggaucaacaccuuguaauggaguggaaggauuuaauuguuauuuuccauuacagagcuauggauuucagcca |
| | | accaauggugugggauaucagccauauagagugguggugcugucuuuugaacugcugcaugcaccugcaacaguguguggaccuaaaaaaucuacaaauuuagugaaa |
| | | aauaaaugugugaauuuuaauuuuaauggauuaacaggaacaggagugcugacagaaucuaauaaaaaauuucugccuuuucagcaguuuggcagagauauugcag |
| | | auaccacagaugcagugagagauccucagacauuagaaauucuggauauuacaccuuguucuuuugggggugugucugugauuacaccuggaacaaauacaucuaauc |
| | | agguggcugugcuguaucaggaugugaauuguacagaagugccaguggcaauucaugcagaucagcugacaccaacauggagaguguauucuacaggaucuaaugugu |
| | | uucagacaagagcaggaugucugauuggagcagaacaugugaauaauucuuaugaaugugauauuccaauuggagcaggcauuugugcaucuuaucagacacagacaa |
| | | auuccccaaggagagcaagaucuguggcaucucagucuauuauugcauacaccaugucucugggagcagaaaauucuguggcauauucuaauaauucuauugcuauuc |
| | | caacaaauuuuaccauuucugugacaacagaaauuuuaccugugucuaugacaaaaacaucuguggauuguaccauguacauuuguggagauucuacagaauguucu |
| | | aaucugcugcugcaguauggaucuuuuuguacacagcugaauagagcuuuaacaggaauugcuguggaacaggauaaaaauacacaggaaguguuugcucaggugaaa |
| | | cagauuuacaaaacaccaccaauuaaagauuuuggaggauuuaauuuuagccagauucugccugauccuucuaaaccuucuaaaagaucuuuuauugaagaucugcu |
| | | guuuaauaaagugacacuggcagaugcaggauuuauuaaacaguauggagauugccugggugauauugcugcaagagaucugauuugugcucagaaauuuaauggac |
| | | ugacagugcugccuccucugcugacagaugaaaugauugcucaguacacaucugcuuuacuggcuggaacaauuacaagcggauggacauuuggagcuggagcugcucu |
| | | gcagauuccuuuugcaaugcagauggcuuacagauuuaauggaauuggagugacacagaauguguuauaugaaaaucagaaacugauugcaaaucaguuuaauucug |
| | | caauuggcaaaauucaggauucucugucuucuacagcuucugcucugggaaaacugcaggauguggugaaucagaaugcacaggcacugaauacucuggugaaacagcu |
| | | gucuagcaauuuuggggcaauuucuucugugcugaaugauauucugucuagacuggauccuccugaagcugaagugcagauugauagacugaucacaggaagacugca |
| | | gucucugcagacuuaugugacacagcagcugauuagagcugcugaaauuagagcuucugcuaaucuggcugcuacaaaaaugucugaaugugugcugggacagucaaa |
| | | aagaguggauuuuuguggaaaaggauaucaucugaugucuuuuccacagucugcuccacauggagugguguuuuuacaugugacauaugugccagcacaggaaaagaa |
| | | uuuuaccacagcaccagcaauuugucaugauggaaaagcacauuuuccaagagaaggaguguuugugucuaauggaacacauugguuugugacacagagaaauuuuu |
| | | augaaccucagauuauuacaacagauaauacauuugugucaggaaauugugauguggugauuggaauugugaauaauacaguguaugauccacugcagccagaacug |
| | | gauucuuuuaaagaagaacuggauaaauauuuuaaaaaucacacaucuccugauguggauuuaggagauauuucuggaaucaaugcaucuguggugaauauucaga |
| | | aagaaauugauagacugaaugaaguggccaaaaaucugaaugaaucucugauugaucugcaggaacuuggaaaauaugaacaguacauuaaauggccuugguacauu |
| | | uggcuuggauuuauugcaggauuaauugcaauugugauggugacaauuauguuauguuguaugacaucauguuguucuuguuuaaaaggauguuguucuugugga |
| | | agcuguuguaaauuugaugaagaugauucugaaccuguguuaaaaggagugaaauugcauuacaca |
| 9 | S protein PP | auguucguguuccuggugcugcugccucugguguccagccagugugugaaccugaccaccagaacacagcugccuccagccuacaccaacagcuuuaccagaggcguguac |
| | (CDS)(V09) | uaccccgacaagguguucagauccagcgugcugcacucuacccaggaccuguuccugccuuucuucagcaacgugaccugguuccacgccauccacguguccggcaccaaug |
| | | gcaccaagagauucgacaaccccgugcugcccuucaacgacgggguguacuuugccagcaccgagaaguccaacaucaucagaggcuggaucuucggcaccacacuggacag |
| | | caagacccagagccugcugaucgugaacaacgccaccaacguggucaucaaagugugcgaguuccaguucugcaacgaccccuuccugggcgucuacuaccacaagaacaac |
| | | aagagcuggauggaaagcgaguuccggguguacagcagcgccaacaacugcaccuucgaguacgugucccagccuuuccugauggaccuggaaggcaagcagggcaacuuc |
| | | aagaaccugcgcgaguucguguuuaagaacaucgacggcuacuucaagaucuacagcaagcacaccccuaucaaccucgugcgggaucugccucagggcuucucugcucug |
| | | gaaccccugguggaucugcccaucggcaucaacaucacccgguuucagacacugcuggcccugcacagaagcuaccugacaccuggcgauagcagcagcggauggacagcug |
| | | gugccgccgcuuacuaugugggcuaccugcagccuagaaccuuccugcugaaguacaacgagaacggcaccaucaccgacgccguggauugugcucuggauccucugagcg |
| | | agacaaagugcacccugaaguccuucaccguggaaaagggcaucuaccagaccagcaacuuccgggugcagcccaccgaauccaucgugcgguuccccaauaucaccaaucu |
| | | gugccccuucggcgagguguucaaugccaccagauucgccucuguguacgccuggaaccggaagcggaucagcaauugcguggccgacuacuccgugcuguacaacuccgcc |
| | | agcuucagcaccuucaagugcuacggcguguccccuaccaagcugaacgaccugugcuucacaaacguguacgccgacagcuucgugauccggggagaugaagugcggcag |
| | | auugccccuggacagacaggcaagaucgccgacuacaacuacaagcugcccgacgacuucaccggcugugugauugccuggaacagcaacaaccuggacuccaaagucggcg |
| | | gcaacuacaauuaccuguaccggcuguuccggaaguccaaucugaagcccuucgagcgggacaucuccaccgagaucuaucaggccggcagcaccccuuguaacggcgugg |
| | | aaggcuucaacugcuacuucccacugcaguccuacggcuuucagcccacaaauggcgugggcuaucagcccuacagagugguggugcugagcuucgaacugcugcaugccc |
| | | cugccacagugugcggcccuaagaaaagcaccaaucucgugaagaacaaaugcgugaacuucaacuucaacggccugaccggcaccggcgugcugacagagagcaacaaga |
| | | aguuccugccauuccagcaguuuggccgggauaucgccgauaccacagacgccguuagagauccccagacacuggaaauccuggacaucaccccuugcagcuucggcggag |
| | | ugucugugaucaccccuggcaccaacaccagcaaucagguggcagugcuguaccaggacgugaacuguaccgaagugcccguggccauucacgccgaucagcugacaccuac |
| | | auggcggguguacuccaccggcagcaauguguuucagaccagagccggcugucugaucggagccgagcacgugaacaauagcuacgagugcgacauccccaucggcgcugg |
| | | aaucugcgccagcuaccagacacagacaaacagcccucggagagccagaagcguggccagccagagcaucauugccuacacaaugucucugggcgccgagaacagcguggcc |
| | | uacuccaacaacucuaucgcuauccccaccaacuucaccaucagcgugaccacagagauccugccuguguccaugaccaagaccagcguggacugcaccauguacaucugcg |
| | | gcgauuccaccgagugcuccaaccugcugcugcaguacggcagcuucugcacccagcugaauagagcccugacagggaucgccguggaacaggacaagaacacccaagaggu |
| | | guucgcccaagugaagcagaucuacaagaccccuccuaucaaggacuucggcggcuucaauuucagccagauucugcccgauccuagcaagcccagcaagcggagcuucau |
| | | cgaggaccugcuguucaacaaagugacacuggccgacgccggcuucaucaagcaguauggcgauugucugggcgacauugccgccagggaucugauuugcgcccagaaguu |
| | | uaacggacugacagugcugccuccucugcugaccgaugagaugaucgcccaguacacaucugcccugcuggccggcacaaucacaagcggcuggacauuuggagcaggcgcc |
| | | gcucugcagauccccuuugcuaugcagauggccuaccgguucaacggcaucggagugacccagaaugugcuguacgagaaccagaagcugaucgccaaccaguucaacagc |
| | | gccaucggcaagauccaggacagccugagcagcacagcaagcgcccugggaaagcugcaggacguggucaaccagaaugcccaggcacugaacacccuggucaagcagcugu |
| | | ccuccaacuucggcgccaucagcucugugcugaacgauauccugagcagacuggacccuccugaggccgaggugcagaucgacagacugaucacaggcagacugcagagccu |
| | | ccagacauacgugacccagcagcugaucagagccgccgagauuagagccucugccaaucuggccgccaccaagaugucugagugugugcugggccagagcaagagagugga |
| | | cuuuugcggcaagggcuaccaccugaugagcuucccucagucugccccucacggcgugguguuucugcacgugacauaugugcccgcucaagagaagaauuucaccaccgc |
| | | uccagccaucugccacgacggcaaagcccacuuuccuagagaaggcguguucguguccaacggcacccauugguucgugacacagcggaacuucuacgagccccagaucauc |
| | | accaccgacaacaccuucgugucuggcaacugcgacgucgugaucggcauugugaacaauaccguguacgacccucugcagcccgagcuggacagcuucaaagaggaacug |
| | | gacaaguacuuuaagaaccacacaagccccgacguggaccugggcgauaucagcggaaucaaugccagcgucgugaacauccagaaagagaucgaccggcugaacgaggug |
| | | gccaagaaucugaacgagagccugaucgaccugcaagaacuggggaaguacgagcaguacaucaaguggcccugguacaucuggcugggcuuuaucgccggacugauugc |
| | | caucgugauggucacaaucaugcuguguugcaugaccagcugcuguagcugccugaagggcuguuguagcuguggcagcugcugcaaguucgacgaggacgauucugag |
| | | cccgugcugaagggcgugaaacugcacuacaca |

Foldon

| | | |
|---|---|---|
| 10 | Foldon | GSGYIPEAPRDGQAYVRKDGEWVLLSTFLGRSLEVLFQGPG |
| | (amino acid) | |
| 11 | Foldon (CDS) | ggaucugguuauauuccugaagcuccaagagaugggcaagcuuacguucguaaagauggcgaauggguauuacuuucuaccuuuuuaggccggucccuggaggugcug |
| | | uuccagggccccggc |

5′-UTR (hAg-Kozak)

| | | |
|---|---|---|
| 12 | 5′-UTR | AACUAGUAUUCUUCUGGUCCCCACAGACUCAGAGAGAACCCGCCACC |

3′-UTR (FI element)

| | | |
|---|---|---|
| 13 | 3′-UTR | CUGGUACUGCAUGCACGCAAUGCUAGCUGCCCCUUUCCCGUCCUGGGUACCCCGAGUCUCCCCCGACCUCGGGUCCCAGGUAUGCUCCC |
| | | ACCUCCACCUGCCCCACUCACCACCUCUGCUAGUUCCAGACACCUCCCAAGCACGCAGCAAUGCAGCUCAAAACGCUUAGCCUAGCCACA |
| | | CCCCCACGGGAAACAGCAGUGAUUAACCUUUAGCAAUAAACGAAAGUUUAACUAAGCUAUACUAACCCCAGGGUUGGUCAAUUUCGU |
| | | GCCAGCCACACC |

A30L70

| | | |
|---|---|---|
| 14 | A30L70 | AAAAAAAAAAAAAAAAAAAAAAAAAAAAAAGCAUAUGACUAAAAAAAAAAAAAAAAAAAAAAAAAAAAAAAAAAAAAAAAAAAAAAA |
| | | AAAAAAAAAAAAAAAAAAAAAAA |

In some embodiments, the RNA polynucleotides described herein comprise the same features as BNT162b2 (summarized below), aside from comprising one or more mutations from a variant S protein identified using a method disclosed herein.

### BNT162b2

| | |
|---|---|
| Structure | m₂^{7,3′-O}Gppp(m₁^{2′-O})ApG-hAg-Kozak-S1S2-PP-FI-A30L70 |
| Encoded | Viral spike protein (S1S2 protein) of the SARS-CoV-2 |
| antigen | (S1S2 full-length protein, sequence variant) |

In some embodiments, the RNA polynucleotides described herein encode two or more epitopes, wherein the epitopes have been derived from variants of concern that have been identified using a method disclosed herein. In some embodiments, the methods described herein are used to identify mutations that substantially elevate the risk of a variant (e.g., mutations that substantially increase the immune escape score and/or the infectivity score). In some embodiments, the RNA polynucleotides disclosed herein encode polypeptides comprising one or more of the mutations that have been determined to substantially elevate the risk of a variant, but do not comprise all the mutations that present in the variant (e.g., do not comprise mutations that are not thought to contribute to the immune escape score or the infectivity score). In some embodiments, the RNA polynucleotides comprise mutations for two or more variants of concern. In some embodiments, the RNA polynucleotides comprise multiple epitopes, e.g., epitopes from multiple variants of concern.

### RNA Delivery Technologies

Provided pharmaceutical compositions (e.g., one or more molecules of RNA encoding a protein from a variant that has been determined to have an elevated risk) may be delivered for therapeutic applications described herein using any appropriate methods known in the art, including, e.g., delivery as naked RNAs, or delivery mediated by viral and/or non-viral vectors, polymer-based vectors, lipid-based vectors, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery RNA molecules described herein.

In some embodiments, one or more RNA molecules can be formulated with lipid particles for delivery (e.g., in some embodiments by intravenous injection).

In some embodiments, lipid particles can be designed to protect RNA molecules (e.g., mRNA) from extracellular RNases and/or engineered for systemic delivery of the RNA to target cells (e.g., dendritic cells). In some embodiments, such lipid particles may be particularly useful to deliver RNA molecules (e.g., mRNA) when RNA molecules are intravenously administered to a subject in need thereof.

In some embodiments, lipid particles comprise liposomes. In some embodiments, lipid particles comprise cationic liposomes

In some embodiments, lipid particles comprise lipid nanoparticles.

In some embodiments, lipid particles comprise lipoplexes.

In some embodiments, lipid particles comprise N,N,N trimethyl-2-3-dioleyloxy-1-propanaminium chloride (DOTMA), 1,2-dioleoyl-sn-glycero-3-phosphoethanolamine phospholipid (DOPE), or both. In some embodiments, lipid particles comprise at least one ionizable aminolipid. In some embodiments, lipid particles comprise at least one ionizable aminolipid and a helper lipid. In some embodiments, a helper lipid is or comprises a phospholipid. In some embodiments, a helper lipid is or comprises a sterol. In some embodiments, lipid particles comprises at least one polymer-conjugated lipid.

RNA lipoplex particles: In some embodiments, RNA molecules described herein may be delivered by liposomal formulations. In some embodiments, negatively charged RNA molecules described herein are complexed with cationic liposomes to form RNA lipoplex particles. In some embodiments, RNA molecules described herein are embedded in a (phospho)lipid bilayer structure within an RNA lipoplex particle. In some embodiments, cationic liposomes can comprise a cationic lipid or an ionizable aminolipid (e.g., ones as described herein) and optionally an additional or helper lipid (e.g., at least one neutral lipid as described herein) to form injectable particle formulations.

In some embodiments, RNA lipoplex particles may be prepared by mixing liposomes with RNA molecules described herein. In some embodiments, liposomes may be obtained by injecting a solution of lipids in ethanol into water or a suitable aqueous phase. In some embodiments, cationic liposomes are stabilized in an aqueous formulation, e.g., as described in WO 2016/046060, the entire content of which is incorporated herein by reference for the purposes described herein. In some embodiments, cationic liposomes may be produced by a method, e.g., as described in WO 2019/077053, the entire content of which is incorporated herein by reference for the purposes described herein.

In some embodiments, spleen targeting RNA lipoplex particles that are useful for delivering RNA molecules described herein are described in WO 2013/143683, the entire content of which is incorporated herein by reference for the purposes described herein. In some embodiments, RNA molecules and positively charged liposomes are mixed such that cationic lipids and RNA are present at a charge ratio of 1.3:2. Such charge ratio is determined to effectively target RNA to the spleen.

In some embodiments, an RNA lipoplex particle comprises a cationic lipid or an ionizable aminolipid (e.g., ones described herein) and an RNA molecule described herein. In some embodiments, such an RNA lipoplex particle may further comprise an additional or helper lipid (e.g., ones described herein). Without wishing to be bound by theory, electrostatic interactions between positively charged liposomes and negatively charged RNA results in complexation and spontaneous formation of RNA lipoplex particles.

In some embodiments where a cationic lipid or an ionizable aminolipid (e.g., ones described herein) and a helper lipid are used, such a cationic lipid or an ionizable aminolipid and such a helper lipid may be present in a molar ratio of 2:1. In some embodiments, a cationic lipid or an ionizable aminolipid may be or comprise DOTMA. In some embodiments, a helper lipid may be or comprise a neutral lipid. In some embodiments, a neutral lipid may be or comprise DOPE.

In some embodiments, RNA lipoplex particles are nanoparticles. In some embodiments, RNA lipoplex nanoparticles can have a particle size (e.g., Z-average) of about 100 nm to 1000 nm or about 200 nm to 900 nm or about 200 nm to 800 nm, or about 250 nm to about 700 nm.

RNA Lipid Nanoparticles (LNPs): In some embodiments, RNA molecules described herein may be delivered by lipid nanoparticle formulations. In some embodiments, RNA lipid nanoparticles may be prepared by mixing lipids with RNA molecules described herein. In some embodiments, at least a portion of RNA molecules are encapsulated by lipid nanoparticles. In some embodiments, at least 90% or higher (including, e.g., at least 95%, 96%, 97%, 98%, 99%, or higher) of RNA molecules are encapsulated by lipid nanoparticles.

In various embodiments, lipid nanoparticles can have an average size (e.g., Z-average) of about 100 nm to 1000 nm, or about 200 nm to 900 nm, or about 200 nm to 800 nm, or about 250 nm to about 700 nm. In some embodiments, lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the particle diameter.

In certain embodiments, RNA molecules (e.g., mRNAs), when present in provided lipid nanoparticles, are resistant in aqueous solution to degradation with a nuclease.

In some embodiments, lipid nanoparticles are cationic lipid nanoparticles comprising one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### Helper Lipids

In some embodiments, a lipid particle for delivery of RNA molecules described herein comprises at least one helper lipid, which may be a neutral lipid, a positively charged lipid, or a negatively charged lipid. In some embodiments, a helper lipid is a lipid that is useful for increasing the effectiveness of delivery of lipid-based particles such as cationic lipid-based particles to a target cell. In some embodiments, a helper lipid may be or comprise a structural lipid with its concentration chosen to optimize particle size, stability, and/or encapsulation.

In some embodiments, a lipid particle for delivery of RNA molecules described herein comprises a neutral helper lipid. Examples of such neutral helper lipids include, but are not limited to phosphotidylcholines such as 1,2-distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, cholesterol, steroids such as sterols and their derivatives. Neutral lipids may be synthetic or naturally derived. Other neutral helper lipids that are known in the art, e.g., as described in WO 2017/075531 and WO 2018/081480, the entire contents of each of which are incorporated herein by reference for the purposes described herein, can also be used in lipid particles described herein. In some embodiments, a lipid particle for delivery of RNA molecules described herein comprises DSPC and/or cholesterol.

In some embodiments, a lipid particle for delivery of RNA molecules described herein comprises at least one helper lipid (e.g., ones described herein). In some such embodiments, a lipid particle may comprise DOPE.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hy droxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(30)-cholest-5-en-3-yloxy]octyl}oxy)—N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)—N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid.

In one embodiment, particles described herein include a polymer conjugated lipid such as a pegylated lipid. The term “pegylated lipid” refers to a molecule comprising both a lipid portion and a polyethylene glycol portion. Pegylated lipids are known in the art.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

In some embodiments, a lipid nanoparticle that is useful in accordance with the present disclosure is or comprises one or more lipids as described in WO 2021/213924, the entire content of which is incorporated herein by reference for purposes described herein. In some embodiments, a lipid nanoparticle that is useful in accordance with the present disclosure is or comprises a lipid nanoparticle composition as described in WO 2021/213924, the entire content of which is incorporated herein by reference for purposes described herein.

### Exemplary Manufacturing Processes

Individual RNA molecules can be produced by methods known in the art. For example, in some embodiments, single-stranded RNAs can be produced by in vitro transcription, for example, using a DNA template. A plasmid DNA used as a template for in vitro transcription to generate an RNA molecule described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, RNA molecules (e.g., ones described herein) can be synthesized in the presence of modified ribonucleotide triphosphates. By way of example only, in some embodiments, N1-methylpseudouridine triphosphate (^{m1}ΨTP) can be used to replace uridine triphosphate (UTP). As will be clear to those skilled in the art, during in vitro transcription, an RNA polymerase (e.g., as described and/or utilized herein) typically traverses at least a portion of a single-stranded DNA template in the 3′→5′ direction to produce a single-stranded complementary RNA in the 5′→3′ direction.

In some embodiments where an RNA molecule comprises a polyA tail, one of skill in the art will appreciate that such a polyA tail may be encoded in a DNA template, e.g., by using an appropriately tailed PCR primer, or it can be added to an RNA molecule after in vitro transcription, e.g., by enzymatic treatment (e.g., using a poly(A) polymerase such as an E. coli Poly(A) polymerase).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increase half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into an RNA molecule ssRNA during transcription (also known as co-transcriptional capping).

Following RNA transcription, a DNA template is digested. In some embodiments, digestion can be achieved with the use of DNase I under appropriate conditions.

In some embodiments, RNA molecules can be purified after in vitro transcription reaction, for example, to remove components utilized or formed in the course of the production, like, e.g., proteins, DNA fragments, and/or or nucleotides. Various nucleic acid purifications that are known in the art can be used in accordance with the present disclosure.

### Provided Pharmaceutical Compositions

The present disclosure provides, among other things, pharmaceutical compositions for delivering to a patient an antigenic viral polypeptide as determined by methods described herein. In some embodiments, a pharmaceutical composition comprises one or more RNA molecules encoding a polypeptide from a viral variant that has been determined to have elevated risk, using a method disclosed here; and lipid particles (e.g., lipoplexes or lipid nanoparticles).

In some embodiments, one or more RNA molecules may be formulated with lipid nanoparticles (e.g., ones described herein) for administration to a patient. Accordingly, in some embodiments, a pharmaceutical composition comprises one or more RNA molecules; and lipid particles (e.g., lipoplexes or lipid nanoparticles), wherein the one or more RNA molecules are encapsulated with the lipid particles (e.g., form an RNA-lipid particle). In some embodiments, an RNA-lipid particle is an RNA-lipoplex particle. In some embodiments, an RNA-lipid particle is an RNA-lipid nanoparticles.

In some embodiments, a pharmaceutical composition comprises multiple RNA molecules, each encoding a different antigen derived from a variant of concern that was identified using a method disclosed herein, wherein each RNA molecule may be present in the pharmaceutical composition in about equimolar amounts.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, M D, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference in its entirety).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In some embodiments, administration is or comprise intramuscular injection.

In some embodiments, pharmaceutical compositions described herein are formulated for intravenous administration. In some embodiments, pharmaceutically acceptable carriers that may be useful for intravenous administration include sterile aqueous solutions or dispersions and sterile powders for preparation of sterile injectable solutions or dispersions.

In some particular embodiments, pharmaceutical compositions described herein are formulated for subcutaneous administration. In some particular embodiments, pharmaceutical compositions described herein are formulated for intramuscular administration.

Therapeutic compositions typically must be sterile and stable under the conditions of manufacture and storage. The composition can be formulated as a solution, dispersion, powder (e.g., lyophilized powder), microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

In some embodiments, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of one or more RNA molecules encapsulated in LNPs, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., one or more RNA molecules encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, a physician or veterinarian could start doses of active ingredients (e.g., one or more RNA molecules encapsulated in lipid nanoparticles) employed in the pharmaceutical composition at levels lower than that required in order to achieve the desired therapeutic effect and gradually increase the dosage until the desired effect is achieved. For example, exemplary doses as described Example 7 may be used in preparing pharmaceutically acceptable dosage forms.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition described herein may further comprises one or more active agents in addition to RNA (e.g., one or more RNA molecules, e.g., one or more mRNA molecules.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the methods and systems disclosed herein can be used to assess SARS-CoV-2 variants, or the pharmaceutical compositions comprise SARS-CoV-2 S protein variants, and immunogenic fragment thereof, or nucleic acids encoding the same (e.g., a vaccine composition comprising an RNA polynucleotide encoding a Spike protein derived from a variant that has been determined to be of risk using a method disclosed herein). In some embodiments, the SARS-CoV-2 variants are any of those variants shown in Table 3 below. In some embodiments, SARS-CoV-2 variants as shown in Table 3 below can be used as reference viral polypeptides in accordance with the present disclosure.

TABLE 3
Mutations found in the spike glycoprotein of SARS-CoV-2
variants of concern and variants with high prevalence.
For lineage B.1.526 two sub-lineages with different
key amino acid exchanges in the RBD are known.

| Lineage | Amino acid substitution |
|---|---|
| B.1.1.7 | del69-70, del144, N501Y, A570D, D614G, P681H, T716I, |
| | S982A, D1118H |
| B.1.351 | L18F, D80A, D215G, del242-244, R246I, K417N, E484K, |
| | N501Y, D614G, A701V |
| P.1 | L18F, T20N, P26S, D138Y, R190S, K417T, E484K, N501Y, |
| | D614G, H655Y, T1027I, V1176F |
| B.1.427/ | S13I, W152C, L452R, D614G |
| B.1.429 | |
| B.1.526 | L5F, T95I, D253G, S477N or E484K, D614G, A701V |
| B.1.160 | S477N, D614G |
| B.1.258 | del69-70, N439K, D614G, N679S, A845S |

del = deletion

In some embodiments, the one or more reference sequences comprise any one of the mutations listed in Table 3.

In some embodiments, the reference protein is the Spike protein from the Wuhan strain of SARS-CoV-2, and corresponding to SEQ ID NO: A, shown below:

(SEQ ID NO: A)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

### Treatment

Also disclosed herein are methods of treating or preventing a disease caused by an infectious agent (e.g., SARS-CoV-2), wherein the method comprises administering a polypeptide (e.g., a Spike protein from a SARS-CoV-2 variant) that has been derived from a variant that has been determined to be of risk using a method disclosed herein, or a nucleic acid encoding the polypeptide. In some embodiments, the method of treatment or prevention comprises administering a pharmaceutical composition disclosed herein. In some embodiments, the method of treatment or prevention comprises administering an LNP or liposome formulation disclosed herein.

In some embodiments, the administered polypeptide or nucleic acid comprises one or more mutations, but not all the mutations detected in a variant that has been determined to be of increased risk (e.g., the mutations that have been determined to most increase the risk of the strain). In some embodiments, the method of treatment or prevention comprises administering two or more polypeptide or nucleic acid sequences (e.g., two or more RNA polynucleotides) that have been derived from a variant that has been determined to be high risk using any one of the methods disclosed herein. In some embodiments, the polypeptides or nucleic acids disclosed herein comprise mutations from multiple high risk variants identified using a method disclosed herein. In some embodiments, polypeptides or nucleic acids comprising mutations from multiple high risk variants offer broader protection (i.e., immune protection against a greater variety of variants) than polypeptides or nucleic acids that comprise mutations from a single variant.

The present invention provides methods and agents for inducing an adaptive immune response against a virus in a subject comprising administering an effective amount of a composition comprising RNA encoding a vaccine antigen described herein (e.g., a coronavirus antigen described herein).

In one embodiment, the methods and agents described herein provide immunity in a subject to coronavirus, coronavirus infection, or to a disease or disorder associated with coronavirus. The present invention thus provides methods and agents for treating or preventing the infection, disease, or disorder associated with coronavirus.

In one embodiment, the methods and agents described herein are administered to a subject having an infection, disease, or disorder associated with coronavirus. In one embodiment, the methods and agents described herein are administered to a subject at risk for developing the infection, disease, or disorder associated with coronavirus. For example, the methods and agents described herein may be administered to a subject who is at risk for being in contact with coronavirus. In one embodiment, the methods and agents described herein are administered to a subject who lives in, traveled to, or is expected to travel to a geographic region in which coronavirus is prevalent. In one embodiment, the methods and agents described herein are administered to a subject who is in contact with or expected to be in contact with another person who lives in, traveled to, or is expected to travel to a geographic region in which coronavirus is prevalent. In one embodiment, the methods and agents described herein are administered to a subject who has knowingly been exposed to coronavirus through their occupation, or other contact. In one embodiment, a coronavirus is SARS-CoV-2. In some embodiments, methods and agents described herein are administered to a subject with evidence of prior exposure to and/or infection with SARS-CoV-2 and/or an antigen or epitope thereof or cross-reactive therewith. For example, in some embodiments, methods and agents described herein are administered to a subject in whom antibodies, B cells, and/or T cells reactive with one or more epitopes of a SARS-CoV-2 spike protein are detectable and/or have been detected.

For a composition to be useful as a vaccine, the composition must induce an immune response against the coronavirus antigen in a cell, tissue or subject (e.g., a human). In some embodiments, the composition induces an immune response against the coronavirus antigen in a cell, tissue or subject (e.g., a human). In some instances, the vaccine induces a protective immune response in a mammal. The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In some embodiments, administration of an immunogenic composition or vaccine of the present disclosure may be performed by single administration or boosted by multiple administrations.

In some embodiments, an amount the RNA described herein from 0.1 μg to 300 μg, 0.5 μg to 200 μg, or 1 μg to 100 μg, such as about 1 μg, about 3 μg, about 10 μg, about 30 μg, about 50 μg, or about 100 μg may be administered per dose. In one embodiment, the invention envisions administration of a single dose. In one embodiment, the invention envisions administration of a priming dose followed by one or more booster doses. The booster dose or the first booster dose may be administered 7 to 28 days or 14 to 24 days following administration of the priming dose.

In some embodiments, an amount of the RNA described herein of 60 μg or lower, 50 μg or lower, 40 μg or lower, 30 μg or lower, 20 μg or lower, 10 μg or lower, 5 μg or lower, 2.5 μg or lower, or 1 μg or lower may be administered per dose.

In some embodiments, an amount of the RNA described herein of at least 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg may be administered per dose.

In some embodiments, an amount of the RNA described herein of 0.25 μg to 60 μg, 0.5 μg to 55 μg, 1 μg to 50 μg, 5 μg to 40 μg, or 10 μg to 30 μg may be administered per dose.

In one embodiment, an amount of the RNA described herein of about 30 μg is administered per dose. In one embodiment, at least two of such doses are administered. For example, a second dose may be administered about 21 days following administration of the first dose.

In some embodiments, the efficacy of the RNA vaccine described herein (e.g., administered in two doses, wherein a second dose may be administered about 21 days following administration of the first dose, and administered, for example, in an amount of about 30 μg per dose) is at least 70%, at least 80%, at least 90, or at least 95% beginning 7 days after administration of the second dose (e.g., beginning 28 days after administration of the first dose if a second dose is administered 21 days following administration of the first dose). In some embodiments, such efficacy is observed in populations of age of at least 50, at least 55, at least 60, at least 65, at least 70, or older. In some embodiments, the efficacy of the RNA vaccine described herein (e.g., administered in two doses, wherein a second dose may be administered about 21 days following administration of the first dose, and administered, for example, in an amount of about 30 μg per dose) beginning 7 days after administration of the second dose (e.g., beginning 28 days after administration of the first dose if a second dose is administered 21 days following administration of the first dose) in populations of age of at least 65, such as 65 to 80, 65 to 75, or 65 to 70, is at least 90%, at least 91%, at least 92%, at least 93%, at least 94%, or at least 95%. Such efficacy may be observed over time periods of up to 1 month, 2 months, 3 months, 6 months or even longer.

In one embodiment, vaccine efficacy is defined as the percent reduction in the number of subjects with evidence of infection (vaccinated subjects vs. non-vaccinated subjects).

In one embodiment, efficacy is assessed through surveillance for potential cases of COVID-19. If, at any time, a patient develops acute respiratory illness, for the purposes herein, the patient can be considered to potentially have COVID-19 illness. The assessments can include a nasal (midturbinate) swab, which may be tested using a reverse transcription-polymerase chain reaction (RT-PCR) test to detect SARS-CoV-2. In addition, clinical information and results from local standard-of-care tests can be assessed.

In some embodiments, efficacy assessments may utilize a definition of SARS-CoV-2-related cases wherein:
Confirmed COVID-19: presence of at least 1 of the following symptoms and SARS-CoV-2 NAAT (nucleic acid amplification-based test) positive during, or within 4 days before or after, the symptomatic period: fever; new or increased cough; new or increased shortness of breath; chills; new or increased muscle pain; new loss of taste or smell; sore throat; diarrhea; vomiting.

Alternatively or additionally, in some embodiments, efficacy assessments may utilize a definition of SARS-CoV-2-related cases wherein one or more of the following additional symptoms defined by the CDC can be considered: fatigue; headache; nasal congestion or runny nose; nausea.

In some embodiments, efficacy assessments may utilize a definition of SARS-CoV-2-related severe cases
Confirmed severe COVID-19: confirmed COVID-19 and presence of at least 1 of the following: clinical signs at rest indicative of severe systemic illness (e.g., RR≥30 breaths per minute, HR≥125 beats per minute, SpO2≤93% on room air at sea level, or PaO2/FiO2<300 mm Hg); respiratory failure (which can be defined as needing high-flow oxygen, noninvasive ventilation, mechanical ventilation, or ECMO); evidence of shock (e.g., SBP<90 mm Hg, DBP<60 mm Hg, or requiring vasopressors); significant acute renal, hepatic, or neurologic dysfunction; admission to an ICU; death.

Alternatively or additionally, in some embodiments a serological definition can be used for patients without clinical presentation of COVID-19: e.g., confirmed seroconversion to SARS-CoV-2 without confirmed COVID-19: e.g., positive N-binding antibody result in a patient with a prior negative N-binding antibody result.

In some embodiments, any or all of the following assays can be performed on serum samples: SARS-CoV-2 neutralization assay; S1-binding IgG level assay; RBD-binding IgG level assay; N-binding antibody assay.

In some embodiments, the methods and agents described herein are administered (in a regimen, e.g., at a dose, frequency of doses and/or number of doses) such that adverse events (AE), i.e., any unwanted medical occurrence in a patient, e.g., any unfavourable and unintended sign, symptom, or disease associated with the use of a medicinal product, whether or not related to the medicinal product, are mild or moderate in intensity. In some embodiments, the methods and agents described herein are administered such that adverse events (AE) can be managed with interventions such as treatment with, e.g., paracetamol or other drugs that provide analgesic, antipyretic (fever-reducing) and/or anti-inflammatory effects, e.g., nonsteroidal anti-inflammatory drugs (NSAIDs), e.g., aspirin, ibuprofen, and naproxen. Paracetamol or “acetaminophen” which is not classified as a NSAID exerts weak anti-inflammatory effects and can be administered as analgesic according to the invention.

In some embodiments, the methods and agents described herein provide a neutralizing effect in a subject to coronavirus, coronavirus infection, or to a disease or disorder associated with coronavirus.

In some embodiments, the methods and agents described herein following administration to a subject induce an immune response that blocks or neutralizes coronavirus in the subject. In some embodiments, the methods and agents described herein following administration to a subject induce the generation of antibodies such as IgG antibodies that block or neutralize coronavirus in the subject. In some embodiments, the methods and agents described herein following administration to a subject induce an immune response that blocks or neutralizes coronavirus S protein binding to ACE2 in the subject. In some embodiments, the methods and agents described herein following administration to a subject induce the generation of antibodies that block or neutralize coronavirus S protein binding to ACE2 in the subject.

In some embodiments, the methods and agents described herein following administration to a subject induce geometric mean concentrations (GMCs) of RBD domain-binding antibodies such as IgG antibodies of at least 500 U/ml, 1000 U/ml, 2000 U/ml, 3000 U/ml, 4000 U/ml, 5000 U/ml, 10000 U/ml, 15000 U/ml, 20000 U/ml, 25000 U/ml, 30000 U/ml or even higher. In some embodiments, the elevated GMCs of RBD domain-binding antibodies persist for at least 14 days, 21 days, 28 days, 1 month, 3 months, 6 months, 12 months or even longer.

In some embodiments, the methods and agents described herein following administration to a subject induce geometric mean titers (GMTs) of neutralizing antibodies such as IgG antibodies of at least 100 U/ml, 200 U/ml, 300 U/ml, 400 U/ml, 500 U/ml, 1000 U/ml, 1500 U/ml, or even higher. In some embodiments, the elevated GMTs of neutralizing antibodies persist for at least 14 days, 21 days, 28 days, 1 month, 3 months, 6 months, 12 months or even longer.

As used herein, the term “neutralization” refers to an event in which binding agents such as antibodies bind to a biological active site of a virus such as a receptor binding protein, thereby inhibiting the viral infection of cells. As used herein, the term “neutralization” with respect to coronavirus, in particular coronavirus S protein, refers to an event in which binding agents such as antibodies bind to the RBD domain of the S protein, thereby inhibiting the viral infection of cells. In particular, the term “neutralization” refers to an event in which binding agents eliminate or significantly reduce virulence (e.g. ability of infecting cells) of viruses of interest.

The type of immune response generated in response to an antigenic challenge can generally be distinguished by the subset of T helper (Th) cells involved in the response. Immune responses can be broadly divided into two types: Th1 and Th2. Th1 immune activation is optimized for intracellular infections such as viruses, whereas Th2 immune responses are optimized for humoral (antibody) responses. Th1 cells produce interleukin 2 (IL-2), tumor necrosis factor (TNFα) and interferon gamma (IFNγ). Th2 cells produce IL-4, IL-5, IL-6, IL-9, IL-10 and IL-13. Th1 immune activation is the most highly desired in many clinical situations. Vaccine compositions specialized in eliciting Th2 or humoral immune responses are generally not effective against most viral diseases.

In some embodiments, the methods and agents described herein following administration to a subject induce or promote a Th1-mediated immune response in the subject. In some embodiments, the methods and agents described herein following administration to a subject induce or promote a cytokine profile that is typical for a Th1-mediated immune response in the subject. In some embodiments, the methods and agents described herein following administration to a subject induce or promote the production of interleukin 2 (IL-2), tumor necrosis factor (TNFα) and/or interferon gamma (IFNγ) in the subject. In some embodiments, the methods and agents described herein following administration to a subject induce or promote the production of interleukin 2 (IL-2) and interferon gamma (IFNγ) in the subject. In some embodiments, the methods and agents described herein following administration to a subject do not induce or promote a Th2-mediated immune response in the subject, or induce or promote a Th2-mediated immune response in the subject to a significant lower extent compared to the induction or promotion of a Th1-mediated immune response. In some embodiments, the methods and agents described herein following administration to a subject do not induce or promote a cytokine profile that is typical for a Th2-mediated immune response in the subject, or induce or promote a cytokine profile that is typical for a Th2-mediated immune response in the subject to a significant lower extent compared to the induction or promotion of a cytokine profile that is typical for a Th1-mediated immune response. In some embodiments, the methods and agents described herein following administration to a subject do not induce or promote the production of IL-4, IL-5, IL-6, IL-9, IL-10 and/or IL-13, or induce or promote the production of IL-4, IL-5, IL-6, IL-9, IL-10 and/or IL-13 in the subject to a significant lower extent compared to the induction or promotion of interleukin 2 (IL-2), tumor necrosis factor (TNFα) and/or interferon gamma (IFNγ) in the subject. In some embodiments, the methods and agents described herein following administration to a subject do not induce or promote the production of IL-4, or induce or promote the production of IL-4 in the subject to a significant lower extent compared to the induction or promotion of interleukin 2 (IL-2) and interferon gamma (IFNγ) in the subject.

### Exemplification

### Example 1: Early Detection of Potential High Risk Variants with In-Silico Simulation and Self-Supervised Language Models

### Summary

The ongoing COVID-19 pandemic is leading to the discovery of hundreds of novel SARS-CoV-2 variants on a near daily basis. While most variants do not impact the course of the pandemic, some variants pose significantly increased risk when the acquired mutations allow better evasion of antibody neutralization in previously infected or vaccinated subjects, or increased transmissibility. Early detection of such high risk variants (HRVs) is paramount for the proper management of the pandemic. However, experimental assays to determine immune evasion and transmissibility characteristics of new variants are resource intensive and time-consuming, potentially leading to delayed appropriate responses by decision makers. The present disclosure provides results of an in silico approach combining spike protein structure modeling, and large protein transformer language models on spike protein sequences to accurately rank SARS-CoV-2 variants for transmissibility factors and immune escape potential. Among other things, the present disclosure documents that transmissibility and immune escape metrics can be combined for an automated Early Warning System (EWS) that is capable of evaluating new variants in minutes and risk monitoring variant lineages in near real time. In early detection mode, the EWS flagged 11 out of 12 variants designated by the World Health Organization (WHO, Alpha-Mu) as potentially dangerous weeks and sometimes months ahead of them being designated as such, demonstrating its ability to help increase preparedness against future variants. Thus, in some embodiments, the present disclosure provides EWS technologies for detection and/or characterization of viral variants, and specifically SARS-CoV-2 variants.

### Introduction

Despite a relatively slow mutation rate in the human coronaviruses, since the emergence of the human coronavirus SARS-CoV-2 in Wuhan in December 2019, over 250,000 different missense variants (as of Nov. 25, 2021) have been identified in the protein-coding viral sequences deposited in the GISAID (Global Initiative on Sharing All Influenza Data) database and associated with multiple lineages. Of these, over 11,300 individual missense mutations have been observed in the Spike protein alone (12,750+, including indels). While most mutations either reduce the overall fitness of the virus, or bear no consequences to its features, some individual or combinations of mutations lead to high risk variants (HRVs), with modified immune evasion capabilities, and/or improved transmissibility. For example, the Alpha (B.1.1.7) variant of concern (VOC) spread widely through higher transmissibility compared with the Wuhan strain, while the Beta (B.1.351) VOC has been shown to be less effectively neutralized by both convalescent sera and antibodies elicited by approved COVID-19 vaccines (Liu et al., 2021). The Delta (B.1.617.2) variant characterized by a high transmissibility led to increased mortality and triggered a renewed growth in cases in countries with both high and low vaccination rates (such as the United Kingdom (Twohig et al., 2021) and India (Singh et al., 2021). Most recently, the more heavily mutated Omicron (B.1.1.529) was amongst the quickest variants to be designated as a VOC by the WHO, due to a combination of widespread dissemination and several concerning mutations in the Spike protein as well as in other proteins (The Technical Advisory Group on SARS-CoV-2 Virus Evolution (TAG-VE), 2021).

Hundreds of new variants are sequenced daily, some of which are added to the GISAID and other databases (Hatcher et al., 2017; Shu and McCauley, 2017). As new sequences continue to naturally emerge, the potential for generation of variants that are both fit (including, e.g., highly transmissible) and highly immune resistant creates a significant challenge for public health authorities. The transmissibility and immune escape potential of a given variant could be assessed experimentally: evaluating one aspect of the fitness (e.g., transmissibility) of variants requires experimental measurements of their binding affinity with its human receptor, angiotensin-converting enzyme 2 (ACE2), which is necessary for host cell infection; assessing immune escape potential requires in vitro neutralization tests involving serum from vaccinated subjects or serum from patients previously infected with other variants of SARS-CoV-2. Both methods are resource intensive and time consuming, and cannot be scaled to properly address the multitude of emergent variants.

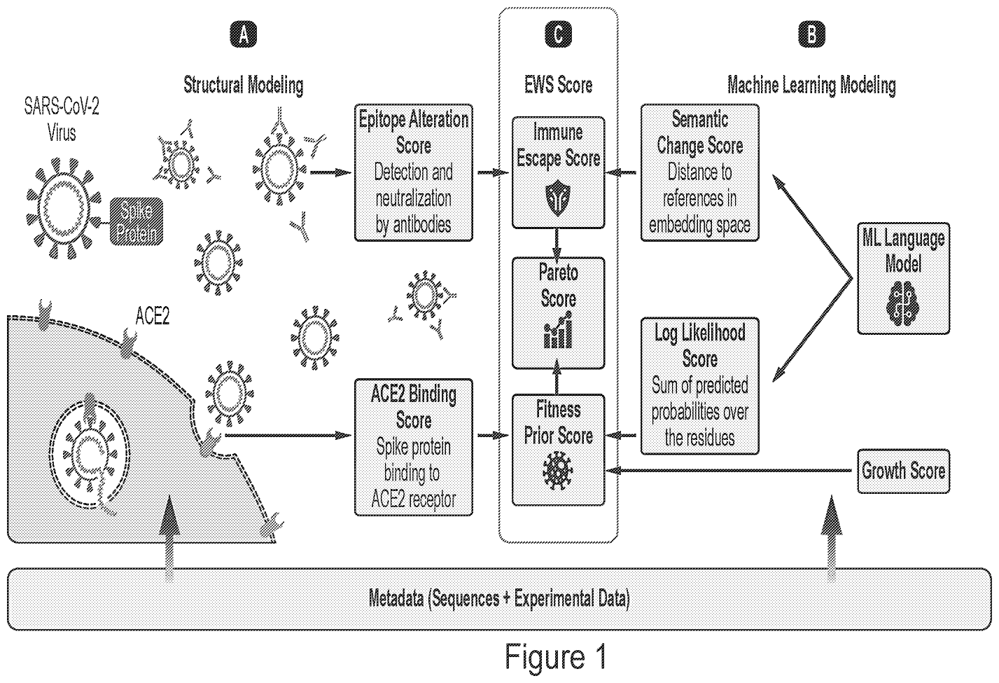

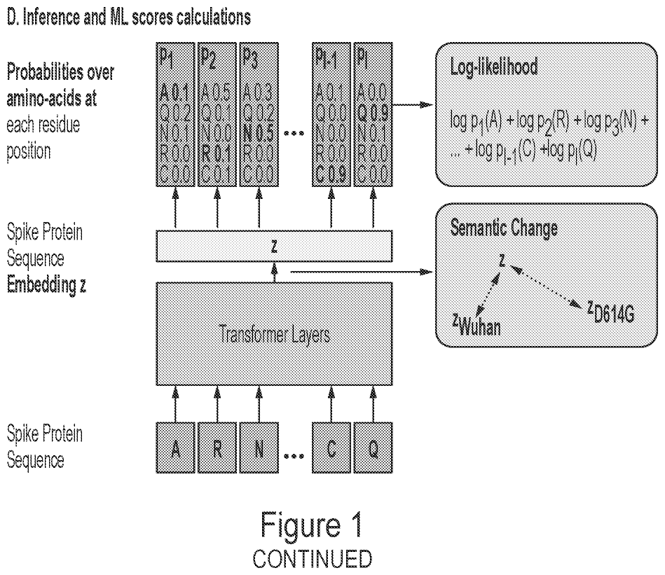

The present disclosure describes and/or utilizes technology to evaluate SARS-CoV-2 variants based on in silico structural modeling and artificial intelligence (AI) language modeling, which technology captures features of a given variant's transmissibility as well as its immune escape properties (FIG. 1). This approach was used to build an Early Warning System (EWS) that trains on the complete (up to a chosen time point) GISAID variants database in less than a day and can score novel variants within minutes. Assessing the risk presented by a novel viral variant is a non-trivial task, as newly emerging High Risk Variants often comprise new sets of mutations, and not all combinations of mutations present in previously identified concerning variants lead to enhanced immune evasion and/or transmissibility. As shown in Example 5, the methods disclosed herein provide superior results (in particular, a better ability to predict variants of concern) than those obtained using standard machine learning methods. The EWS is fully scalable as new variant data become available, allowing for the continuous risk monitoring of variant lineages and has flagged HRVs weeks and sometimes months earlier than their designation as such by the WHO, providing an opportunity to shorten the response time of health authorities.

### Results

### In Silico Prediction of Immune Escape Potential

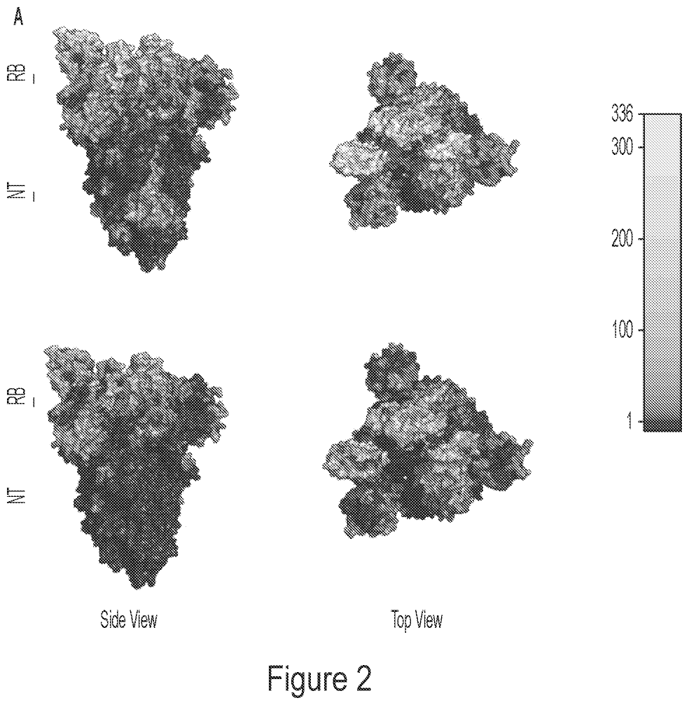

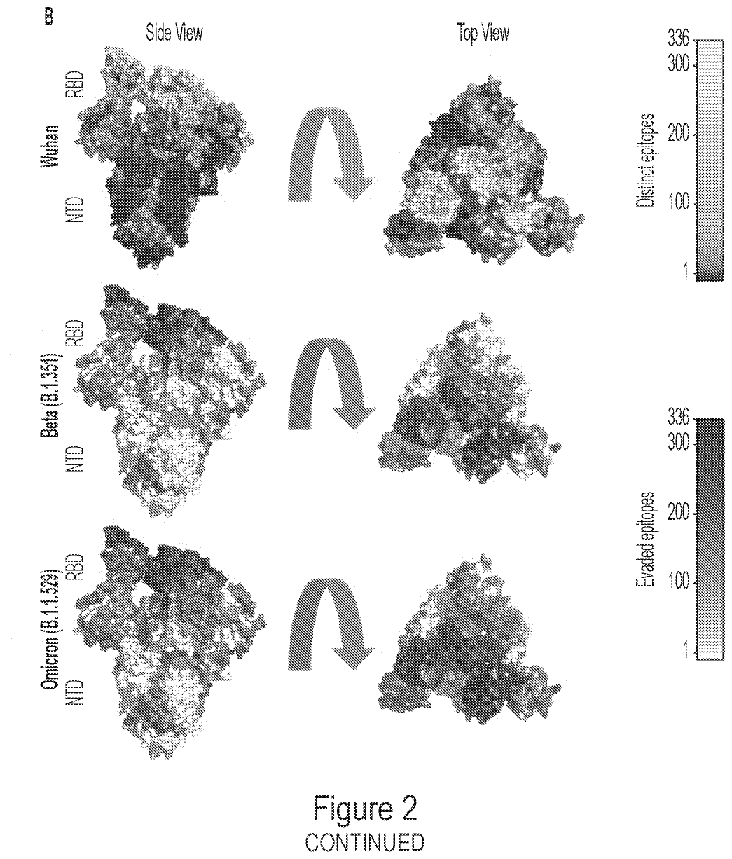

Mutations in the spike (S) protein, especially the receptor-binding domain (RBD), may play a role in the heightened resistance to antibody-mediated neutralization of new SARS-CoV-2 variants (Weisblum et al., 2020). To evaluate the impact of said mutations on humoral immune evasion, the 336 binding epitopes observed in 310 previously resolved structures of neutralizing antibodies (nAbs) (Barnes et al., 2020; Ju et al., 2020; Dejnirattisai et al., 2021; Yan et al., 2021) were mapped onto the S protein based on publicly available resolved 3D structures (Table 6). An overlay of all nAb:S protein interaction interfaces was used to generate a color-coded heat-map, indicating which surface exposed amino acids are located in high epitope density regions (FIG. 2). The number of known nAbs whose binding epitope is affected by a distinct SARS-CoV-2 variants' mutations was defined as the epitope alteration score (EAS).

While this score can be used as a first proxy to evaluate escape from humoral immunity, it may be limited by its dependence on the quantity of available antibody structure data. Herein, deep learning language models were used to leverage information from hundreds of thousands of SARS-CoV-2 S protein sequences deposited to the GISAID database. It was recently demonstrated that these algorithms have the ability to capture biological properties of proteins through the unsupervised learning on large amounts of biological data (Elnaggar et al., 2020; Rives et al., 2021; Steinegger et al., 2019). At the time of inference, a language model returns the predicted probability distribution of the twenty natural amino acids for each position in the protein, thus leveraging underlying biology of the large amount of sequences seen during training from an evolutionary point of view. Hie et al. (Hie et al., 2021) showed that language models trained on a dataset of proteins can be used to assess the risk of a viral variant. This risk was measured through two proxies named grammaticality as a measure for fitness and semantic change to assess antigenic variation. In the approach described herein, the recurrent neural networks used in (Hie et al., 2021) were replaced with attention-based models, namely transformers (Vaswani et al., 2017), hence replacing the auto-regressive way of training the model used in (Hie et al., 2021) by the BERT (Bidirectional Encoder Representations from Transformers) protocol. Even though the GISAID dataset contains hundreds of thousands of spike protein sequences, it is limited to SARS-CoV-2. To learn more general features of protein sequences and address currently unseen viral variants, one would need to use more comprehensive protein sequence resources, such as the UniProtKB database that includes hundreds of millions of protein sequences (UniProt Consortium, 2019). To benefit from this large volume of available data, the model was first pre-trained over the large collection of varied proteins included in UniProt50 and/or UniRef100 (non-redundant sequence clusters of UniProKB and selected UniParc records) and then fine-tuned over S protein sequences. The transformer model has been re-trained every month on the variants registered in GISAID (122,466 unique S sequences on 3rd of September 2021 vs. 4,172 S sequences in Hie et al. (Hie et al., 2021)). The semantic change calculation was extended by computing it to estimate the change with respect to the wild type and from the D614G mutation to take into account this mutant that largely replaced the Wuhan strain. The same transformer model was leveraged to calculate the log-likelihood of the input sequence: the likelihood of occurrence of a given input sequence. The higher the log-likelihood of a variant, the more probable is the variant to occur from a language model perspective. In particular, the log-likelihood metric supports substitutions, insertions and deletions without requiring a reference.

In vitro pseudovirus neutralization test (pVNT) assays were used to validate the immune escape in silico metrics: semantic change and epitope score. The cross-neutralizing effect of n≥12 BNT162b2-immune sera was assessed against vesicular stomatitis virus (VSV)-SARS-CoV-2-S pseudoviruses bearing the spike protein of n=17 selected variants of interest (Table 4) (Muik et al., 2021; Sahin et al., 2021). Both the epitope score and the semantic change score correlate positively with the calculated 50% pseudovirus neutralization titer (pVNT₅₀) reduction (FIG. 2.A; Pearson r=69.5% and 59.3%, respectively). Of note, an average of both in silico scores (summarized as the ‘immune escape score’) exhibits a slightly stronger correlation with the observed reduction in neutralizing titers (Pearson r=70.8%).

Relatively high immune escape score observed for newly occurring variants might be due to the selection in infected individuals i.e. intrapatient evolution driving the generation of variants (cite SARS-CoV-2 Variants in Patients with Immunosuppression).

### In Silico Estimation of Infectivity or Fitness

The immune escape score predicts if a given viral variant may evade neutralization by the immune system, but it does not capture protein changes that either enhance efficacy of viral cell entry, or negatively impact its structure or function. Capturing the full transmissibility potential of the virus (fitness, also referred to herein as infectivity) may involve many complex dynamics. Described herein are at least three informative factors contributing toward it: ACE2 binding score, log-likelihood score and growth.

One determinant of viral spread is the effectiveness with which virus particles can attach to and invade target host cells. This characteristic may be especially important when considering individuals without pre-existing immunity or viral variants which are able to better evade immune responses. To infect the human host cell, the RBD of the viral S protein associates with ACE2, the cellular receptor for SARS-CoV-2. Infectivity was assessed based on the predicted impact of sets of mutations on the binding affinity of the variant S protein to the human ACE2 receptor, here referred to as the ACE2 binding score. The interaction between a variant S protein and the ACE2 protein was computed through repeated, fully flexible, in silico docking experiments, allowing for unbiased sampling of the binding landscape. In order to reduce the required computational resources, spike protein modeling was restricted to its RBD domain, i.e. the domain known to directly bind to the ACE2 receptor. The median difference in solvent accessible surface between bound and unbound states, which acts as a proxy for complex affinity, was used to calculate ACE2 binding score. Compared to conventional energy metrics, surface area is less sensitive to local optimization pitfalls (e.g. side chain packing), and it is more robust across multiple samples, and generally requires less computational resources to compute accurately.

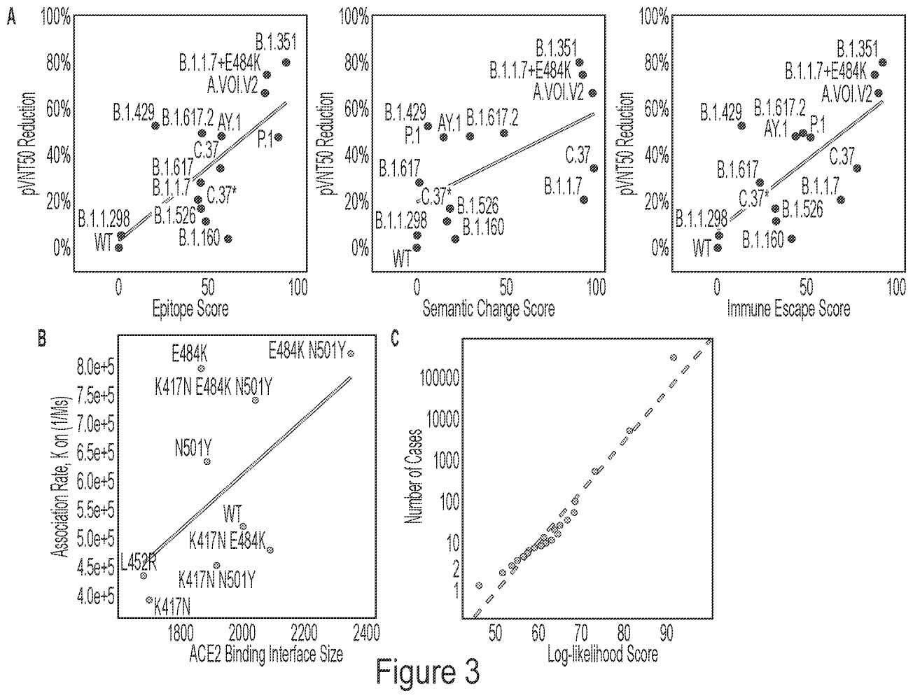

In order to assess the validity of the ACE2 binding score, the simulation results were compared with in vitro results reported by Tanaka et al. (Tanaka et al., 2021). A biolayer interferometry (BLI) kinetic analysis was performed to measure the association rate (kₒₙ) for targeted sets of mutations and showed that association rate correlates with infectivity. The ACE2 binding score showed strong correlation with the association rate (kₒₙ) for targeted sets with a Pearson correlation coefficient of 0.8. (FIG. 3B).

Another aspect that partially models the fitness of a variant, is how similar a given variant is to the other variants which have been known to grow rapidly. Effective assessment of such similarity may not be achievable by simple sequence comparison, due to epistatic interactions between sites of polymorphism, in which certain mutation combinations enhance fitness while being deleterious when they occur separately. The same trained transformer model described previously was leveraged to calculate the log-likelihood of the input sequence. From a language model perspective, the higher the log-likelihood of a variant, the more probable is the variant to occur. In particular, the log-likelihood metric supports substitutions, insertions, and deletions with requiring a reference sequence to measure against, unlike the grammaticality of (Hie et al., 2021) that requires a reference sequence. The language model disclosed herein, was not provided with explicit sequence count data in the training phase, yet on average assigned higher log-likelihood values to sequences with higher actual observed count (FIG. 2C). High log-likelihood may indicate features common in the general variant population, which are likely to be fitness-related, thus allowing strains harboring these to sustain additional such mutations. The present disclosure utilized a log-likelihood of a newly observed sequence as predictive of its expected frequency in population.

Metrics discussed above may not capture the entirety of factors affecting frequency of viral variants. Additionally, log-likelihood is a metric measuring similarity to already known, rapidly increasing samples. By its nature, it cannot accurately assess variants which exhibit completely new sequence features, until these features are observed more often. The present disclosure utilizes an infectivity metric that includes growth, an empirical term of the quantified change in the fraction of observed sequences in the database that a variant in question comprises. One feature of growth is that in this work it is considered by mutations on the RBD only. However fitness of the virus may also be dependent on and/or influenced by mutations in other proteins of the virus. Variants which are increasing in prevalence may be considered to be more imminently interesting than those which do not.

### Combining Infectivity (Fitness Prior) and Immune Escape Scores to Continuously Monitor High Risk Variants

Different selective pressures on virus evolution lead to variants with high immune escape and fitness (e.g., infectivity), since a virus must remain evolutionarily competent or preserve evolutionary fitness to successfully spread. A system that keeps track of immune escape and infectivity factors can monitor (e.g., can continuously monitor) high risk variants (HRVs) on a near real-time basis, since new sequences can be evaluated and added to the data pool in minutes.

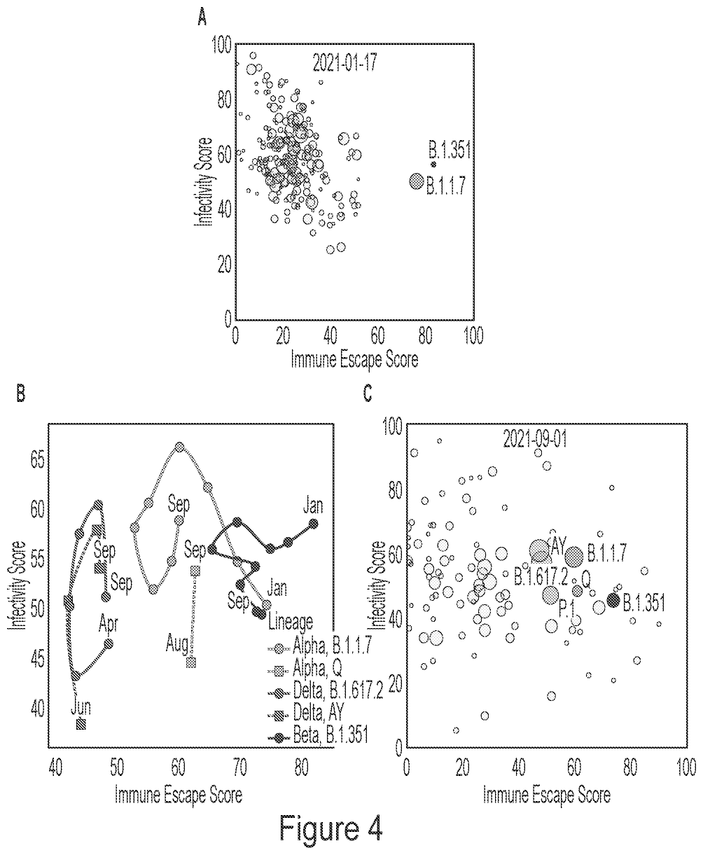

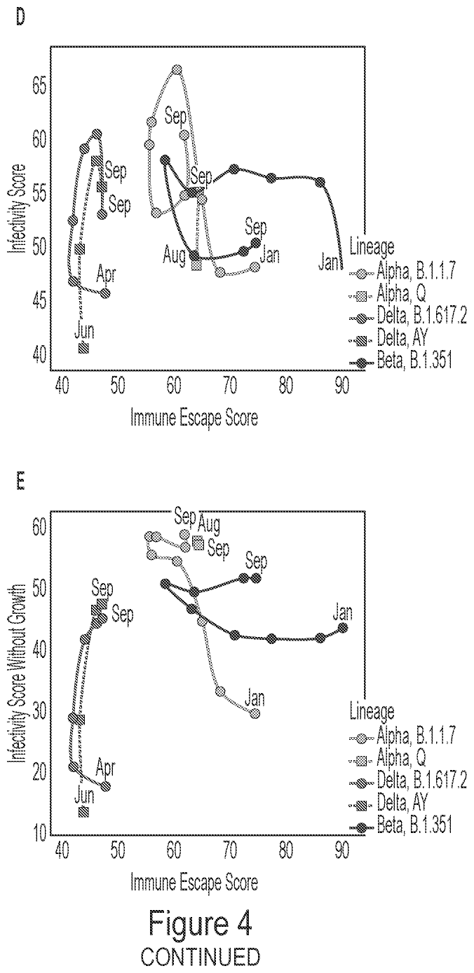

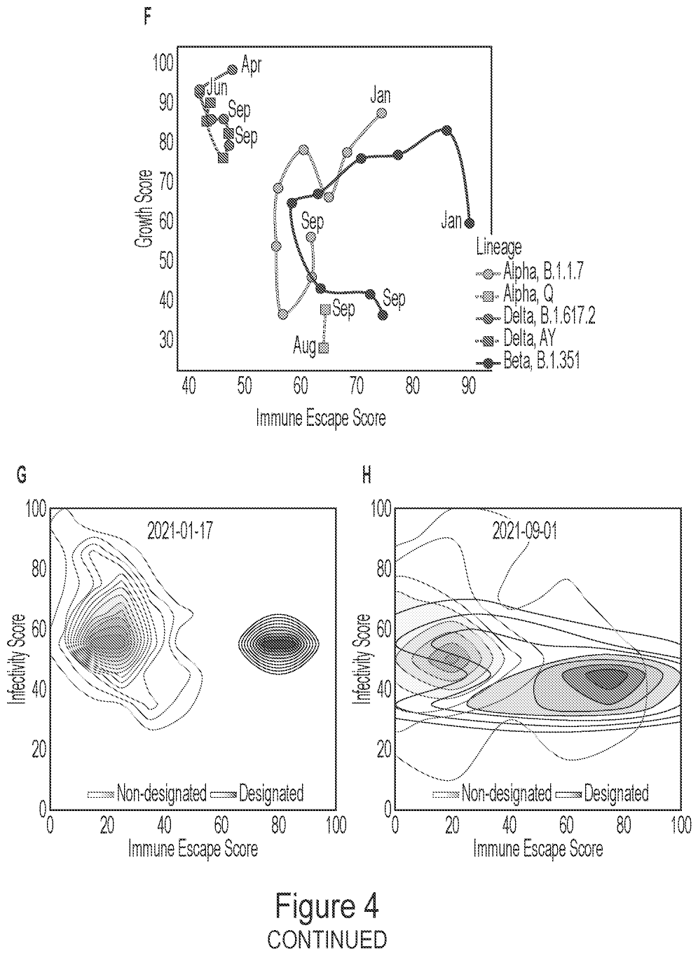

The ranking of any sequence, and consequently-lineage, depends on the other, circulating sequences. As seen in FIG. 4B, variants of concern start off relatively far into the upper-right corner (i.e. are comparatively highly immune escaping and have satisfactory infectivity score for their immune escape value). Then, on the basis of a fixed variant, new more diverse variants emerge, some of which may be more immune escaping, but most of which are less fit and thus less observed in nature. Newly emerging variants diversity over time, as there are more circulating sequences observed. Thus the aggregated immune escape score decreases, for example, due to increased competition from other variants, while infectivity score (based partially on prior observations)-increases for truly fit variants (Alpha: B.1.1.7 and Q lineages, and Delta: B.1.617.2 and AY lineages). Lineages such as Beta or Gamma are observed to be progressively decrease in aggregated infectivity, closely recapitulating their lack of global growth, despite continued prevalence. The effect of perceptible global growth of B.1.351 (Beta) in April 2021, as well as its drop in prevalence and acquisition of further diversifying mutations, are all visible in the plot. The case of B.1.351 (Beta) illustrates a variant which is-according to the data and statistical models-unlikely to regain global significance. Simultaneously, one needs to observe the effects of fitness-enhancing events (either mutations, or emergence of evolutionary niche), such as the increase in metrics for Alpha lineages in Summer 2021, which was possibly due to the competitive effect of B.1.617.2 emergence. This evolutionary pressure could have been one of the factors behind the near eradication of B.1.1.7 and the remaining sequences are under evolutionary pressure to adapt to the changing circumstances, and while currently not significant globally, still pose a tangible threat.

To jointly score the relative risks of variants using immune escape potential and fitness (e.g., infectivity), an optimality score, termed Pareto score, was used to assess variants. The Pareto score is a mathematically robust way to identify lineages that are both immune escaping and infectious, and captures the relative evolutionary advantage of a given strain (see Methods section for calculation details). For each lineage, as defined by the Pango nomenclature system (Rambaut et al., 2020), scores were calculated by averaging the scores of the individual sequences belonging to a given lineage. A high Pareto score at a given time for a specific lineage indicates that only a few other lineages have higher scores for fitness (e.g., infectivity) and immune escape at that time.

In order to validate that the Pareto score separates WHO designated variants from non-designated variants, Welch's t-tests were conducted over the registered variants population respectively on the 17th of January 2021 and on the 1st of September 2021 and p-values=2e-6 and 3e-2 were reported. The same study was conducted every week from January 2020 to the end of August 2021. The null hypothesis can be rejected with a p-value<alpha=0.05, 31 out of 32 weeks, thus demonstrating that the Pareto score can separate designated from non-designated variants continuously through time.

Kernel density estimates (KDE) conducted on Jan. 17, 2021 and Sep. 1, 2021 also demonstrate clear separability between WHO designated variants and non-designated ones (FIG. 4D-E). Importantly they suggest that immune escape significantly contributes to this separability, and in relative terms more so than the infectivity score. See, e.g., Table 7.

### Detection of Potentially High Risk Variants Prior to Substantial Spread in Population

Experimental assays aiming to determine a given variant's immune evasion and fitness (e.g., infectivity) are time and resource intensive. Available data show that approximately thousands of new variants are emerging every week at an increasing rate (on average ˜250 per week in September 2020, 7,000 in August 2021, and 10,000 in October 2021). Moreover, this number is likely an underestimate given limited viral sequencing and data deposition in many countries. It is therefore not feasible for health authorities to perform preventive experimental (e.g., in vitro) assessments whenever a new variant is identified, despite the benefits of a proactive stance detecting HRVs before their spread.

As seen in the previous section, the utilized EWS immune escape score helps separate WHO designated variants from non-designated variants and has demonstrated a significant correlation to in vitro neutralization test results. In addition, the immune escape score is computed from sequence alone and unlike the described infectivity score does not require growth metrics, which are not available when a novel variant gets sequenced. This means that an early detection version of the described system, operating based on immune escape score alone, could spot dangerous variants or specifically HRVs. Increasing vaccination rates world-wide put an added emphasis on immune evasion as a risk factor in newly sequenced variants; in this context, immune escape may be particularly (and potentially solely) useful for early HRV detection.

Moreover, it was recently proposed that viral evolution in immunocompromised patients generates intrapatient viral variants with increased immune evasion, rather than increased fitness and constitutes a significant factor contributing to variant spread (Corey et al., 2021; Weigang et al., 2021). Some of the new variants reside on long branches of phylogenetic tree, which suggests they could have undergone an extensive intrapatient evolution enabled by the immunocompromised status of the host. These results, together with increased vaccination rates world-wide, put an added emphasis on immune evasion as a key risk factor in newly emerging variants, provided further motivation to use an approach that uses only immune escape score to detect HRVs.

A systematic analysis was conducted where for every week between Sep. 16, 2020 and Sep. 2, 2021 the EWS ranked all new sequences on immune escape to compile a weekly flagged HRV watch-list. The models were trained on variants up to the previous month's start date and any other data used was prior to the analysis date. To assess the system's sensitivity, the ability of the algorithm to detect the 12 variants designated by WHO (Alpha, Beta, Gamma, Delta, Epsilon, Eta, Zeta, Lambda, Iota, Kappa and Mu) was assessed.

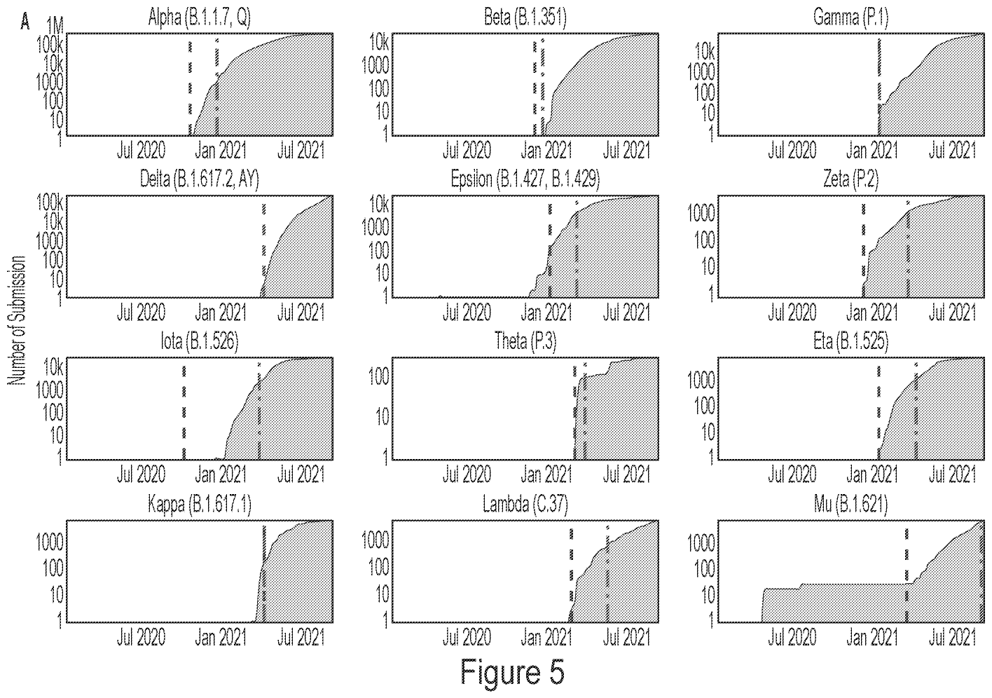

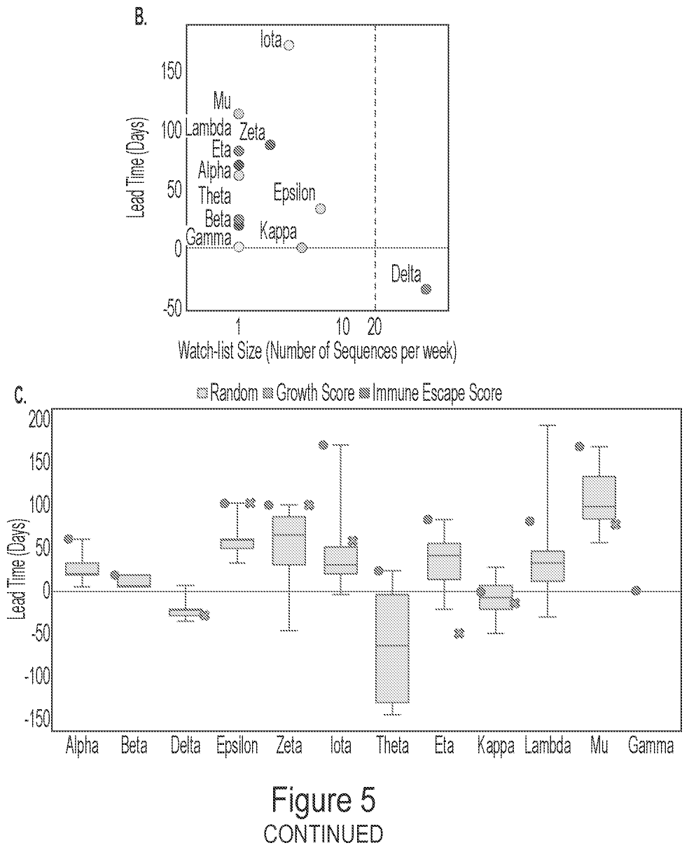

When using a weekly watch-list with a size of 20 variants (less than 1% of the weekly average of new variant sequences), EWS flagged 11 WHO designated variants out of 12 (FIG. 5. A), with an average 72 days of lead time before these were designated as such by the WHO. On average only 0.5% of cases of that variant were already recorded at the time of their detection by the EWS (19 sequences on average), to be contrasted with the WHO announcements that happened on average when 20% of cases for that variant were already recorded (2,000 sequences on average) (Table 5). Different watch-list sizes ranging from 1 up to 100 variants were assessed to evaluate detection sensitivity (FIG. 4.B). The number of named variants detected remained stable when varying the weekly watch list size between 10 and 100. While a longer list compromises specificity, it leads to an increase in the detection lead time (the number of days ahead of WHO designation) (FIG. 6).

While the system as described in this Example did not accurately pinpoint the emergence of the B.1.617.2 Delta family of variants, the system can be modified to account for mutations that may contribute to abrogation of O-glycosylation, which further enables furin cleavage. In particular, Delta is known to be neutralized by vaccines (Liu et al., 2021α) and its global prevalence can be attributed to other fitness-enhancing factors. These factors, such as P681R mutation (Zhang et al., 2021), which abrogates O-glycosylation, thus further enabling furin cleavage. Delta variants also first emerged in India, a vast country with a diverse population and relatively limited sequencing capabilities, hence available samples may have been insufficient to fully describe the epidemiological landscape in time. Government regulations prohibiting the export of biological data out of the country may have also further restricted sequence data from reaching global databases like GISAID. Thus, as more samples and biological data become available, thy system can be improved.

Strikingly, WHO-designated variants Alpha, Beta, Theta, Eta, and Gamma are detected on the same week they are first reported, even in the extreme case where the weekly watch-list allows only one variant, meaning they were the top scoring sequences among all emerging variants that week (FIG. 4.B). In addition, using a larger weekly watch-list of 20 variants, Epsilon, Iota, Zeta, Eta, and Mu are also detected in the same week they are first reported, in addition to the previously mentioned WHO-designated variants.

One can consider growth score as a plausible metric that requires neither AI nor simulation to early detect HRVs with yet better than random results (FIG. 4.C). However, the immune escape score implemented in EWS outperforms the growth score across all of the variants where a comparison is available (7/7), in terms of lead time ahead of WHO designation. The growth score also fails to detect Delta ahead of time despite the established fitness of this variant, which may be another consequence of incomplete or delayed sequencing data (FIG. 4C).

### Discussion

Validation of the immune escape and infectivity scores using published and newly generated data, showed that the combination of structural simulations, AI and biological nucleic sequencing of the SARS-CoV-2 allows continuous risk monitoring and sensitive early detection of HRVs. The Early Warning System can sensitively detect HRVs months ahead of the official WHO designation, sometimes within the same week a sequenced variant enters the database. Specifically, EWS's flagging of the Delta only after its designation by the WHO, with a significant underrepresentation of the lineage in GISAID, emphasizes the importance of extensive, robust and timely sequencing of SARS-CoV-2 genomic samples (e.g., in a potential region and/or globally).

Combining comprehensive sequencing with structural modeling and AI can provide unprecedented insights into the Covid-19 pandemic which could be harnessed, e.g., by public health authorities and governments worldwide to increase their early preparedness to HRVs and potentially alleviate the associated human and economic costs.

### Materials and Methods.

The methodologies described above are described in greater detail in the following sections.

### Variant Notations

As used herein, a “variant” of a Spike protein refers to a protein sequence of a coronavirus' spike protein that differs from the original Wuhan spike protein (also referred to herein as the wild type spike protein). Variants are represented in terms of their mutations with respect to the Wuhan strain. For instance, the notation A12F H156K represents a protein sequence obtained when replacing the amino acid A at position 12 by F and when replacing H by K at position 156 in the Wuhan spike protein sequence.

### VSV-SARS-CoV-2 S Pseudovirus Neutralization Assay

A recombinant replication-deficient VSV vector that encodes green fluorescent protein (GFP) and luciferase (Luc) instead of the VSV-glycoprotein (VSV-G) was pseudotyped with SARS-CoV-2 spike (S) protein derived from either the Wuhan reference strain (NCBI Ref: 43740568) or variants of interest according to published pseudotyping protocols (Berger Rentsch and Zimmer, 2011; Rives et al., 2021). The mutations found in S of the VOCs are listed in Table 4. In brief, HEK293T/17 monolayers transfected to express SARS-CoV-2 S with the C-terminal cytoplasmic 19 amino acids truncated (SARS-CoV-2-S[CΔ19]) were inoculated with the VSVΔG-GFP/Luc vector. After incubation for 1 hour at 37° C., the inoculum was removed, and cells were washed with PBS before medium supplemented with anti-VSV-G antibody (clone 8G5F11, Kerafast) was added to neutralize residual input virus. VSV-SARS-CoV-2 pseudovirus-containing medium was collected 20 hours after inoculation, 0.2 μm filtered and stored at −80° C.

For pseudovirus neutralization assays, 40,000 Vero 76 cells were seeded per 96-well. Sera were serially diluted 1:2 in culture medium starting with a 1:15 dilution (dilution range of 1:15 to 1:7,680). VSV-SARS-CoV-2-S pseudoparticles were diluted in culture medium to obtain ˜200 transducing units (TU) in the assay. Serum dilutions were mixed 1:1 with pseudovirus for 30 minutes at room temperature prior to addition to Vero 76 cell monolayers and incubation at 37° C. for 24 hours. Supernatants were removed, and the cells were lysed with luciferase reagent (Promega). Luminescence was recorded, and neutralization titres were calculated by generating a four-parameter logistical fit of the percent neutralization at each serial serum dilution. The pVNT₅₀ is reported as the interpolated reciprocal of the dilution yielding a 50% reduction in luminescence. If no neutralization yielding a 50% reduction in luminescence was observed, an arbitrary titer value of 7.5 (half of the limit of detection [LOD]) was reported.

### Language Modeling

The domain of Natural Language Processing (NLP) has experienced several breakthroughs in the past years. The emergence of recurrent and attention-based deep neural networks led to impressive results for text generation and translation. Recently, this technology has been leveraged to learn the language of biology (Elnaggar et al., 2020; Rives et al., 2021). It works with a simple analogy where protein sequences are considered as sentences and the amino-acids as words. The models are trained on large datasets of known protein sequences in an unsupervised manner. In other words, there is no need to label the data and any newly registered protein sequence can be exploited.

Information about protein properties is stored at two positions inside the model once it is trained. On one side, the probabilities returned by the model indicate how likely this sequence is to be natural/viable/feasible. On the other hand, the outputs of the model's layers and notably the last layer provide a high dimensional representation for each sequence, referred to herein as embedding of the protein. The embedding of the protein contains information about the protein properties and can be used either directly or to train a classification or regression model. Recently, (Meier et al.) demonstrated that these models also capture the effects of mutations on protein function (Meier et al., 2021).

### Model Architecture

In the methods utilized herein, the input of the model consists of the sequence characters corresponding to the amino acids forming the protein. Each amino acid is first tokenized, i.e. mapped to their index in the vocabulary containing the 20 natural amino acids (+X), and then projected to an embedding space. The sequence of embeddings is then fed to the Transformer model (20) consisting of a series of blocks, each composed of a self-attention operation followed by a position-wise multi-layer network (FIG. 6).

Self-attention modules explicitly construct pairwise interactions between all positions in the sequence which enable them to build complex representations that incorporate context from across the sequence. Because the self-attention operation is permutation-equivariant, a positional encoding must be added to the embedding of each token to distinguish their position in the sequence.

### Self-Supervised Training

Given a large database of protein sequences, the model can be trained using the masked language modeling objective presented in [31]. Each input sequence is corrupted by replacing a fraction of the amino acids with a special mask token. The network is then trained to predict the missing tokens from the corrupted sequence. In practice, for each sequence x, a set of indices i∈M are randomly sampled, for which the amino acid tokens are replaced by a mask token, resulting in a corrupted sequence \hat{x}. During pre-training the set M is defined such that 15% of the amino-acids in the sequence get corrupted. When corrupted an amino-acid has 10% to be replaced by another randomly selected amino-acid and 80% being masked. During fine-tuning these probabilities do not change, however only 3% of the amino-acids in the sequence get corrupted. This probability was selected in order to enable the model to become more accurate for spike protein sequences while keeping its performance on varied sequences from Uniprot50. The training objective corresponds to the negative log-likelihood of the true sequence at the corrupted positions.

$${L}_{θ} ( \hat{x} \vert x ) = - {\sum }_{i ∈ M} log p ( {x}_{i} \vert \hat{x} )$$

To minimize this loss, the model must learn to identify dependencies between the corrupted and uncorrupted elements of the sequence. Consequently, the learned representations of the proteins, taken as the average of the embeddings of each amino acid (FIG. 6), must successfully extract generic features of the biological language of proteins. These features can then be used to fine-tune the model on downstream-tasks.

In this work, the transformer model from (Rives et al, 2021) (esm1_t34_670M_UR100) was used, which was trained using the aforementioned procedure on the UniRef100 dataset (Suzek et al., 2007), containing +277M representative sequences. The pre-trained model was then fine-tuned every month on all the spike protein sequences registered in the GISAID data bank at the training date.

### Data

The genomic sequences and spike protein sequences were collected from GISAID. For each spike protein sequence, the missing amino acids were filled using the next known amino acid and the lineage assignment was performed using PANGOLIN (O'Toole et al., 2021). Mutations with respect to the wild type were calculated using Clustal Omega (O'Toole et al., 2021) and HH-suite (Steinegger et al., 2019).

The GISAID dataset is imbalanced towards some lineages that have been more prevalent and because certain regions have performed more sequencing than others. To mitigate this bias in the dataset during training, the importance of each sequence was weighed differently in the loss calculation. The importance of a sequence is defined as

$${s}_{} ∝ {\sum }_{( s , l ) ∼ D} {log}_{10} ( {c}_{s} ) + {log}_{10} ( {c}_{( s , l )} ) + {log}_{10} ( {c}_{l \vert s} )$$

where the values cₛ and c_{s,l} are the numbers of occurrences in the dataset of the sequence s and the sequence-laboratory pair (s, l), respectively. The value c_{l|s} corresponds to the number of laboratories having reported sequence s, which measure the prevalence across regions of the variant.

Gradient descent is used to minimize the loss function. The Adam optimizer (Kingma and Ba, 2014) was used in the method described herein, which uses a learning rate schedule. The fine-tuning started with a warm-up period of 100 mini-batches where the learning rate increased linearly from 10⁻⁷ to 10⁻⁵. After the warm-up period, the learning rate decreased following 10⁻⁶ √x where x represents the number of mini-batches.

### Inference and ML Scores Calculations

Once fine-tuned, the model can be used to compute the semantic change and the log-likelihood to characterize a spike protein sequence s. The output of the last transformer layer is averaged over the residues to obtain an embedding z of the protein sequence. The embedding of the Wuhan strain z_{wuhan} and the embedding of the D614G variant z_{D614G} are computed once for all. In some embodiments, the semantic change is computed as the sum of the Euclidean distance between the z and z_{wuhan} the Euclidean distance between z and z_{D614G}. More formally, the semantic change is computed as:

$$Δ z = {‖ z - ‖}_{2} + {‖ z - {z}_{D 614 G} ‖}_{2}$$

where ∥⋅∥₂ is the Euclidean distance.

The log-likelihood can be computed from the probabilities over the residues returned by the model. It is calculated as the sum of the log-probabilities over all the positions of the spike protein amino-acids.

Given a variant's sequence s, the fine-tuned neural network provides a discrete probability distribution over all amino acids A for each position i:

$${\{ p ( {s}_{i} = a \vert s ) \}}_{a ∈ A}$$

where p(sᵢ=a|s) is the probability that the i-th position is amino acid a. The variant's log-likelihood metric is therefore defined as

$$l ( s ) = {\sum }_{i} log p ( {s}_{i} = {s}_{i} \vert s ) ,$$

which measures the likelihood of having the same variant given itself. In particular, the proposed log-likelihood metric supports substitution, insertion and deletion without requirement of a reference.

### ACE2 Binding Score

279 receptor-binding domain (RBD) differentiated variants, including the wide type, were selected for in-silico simulation. For each variant, a putative structure was generated, from which at least 500 structures were generated through a conformational sampling algorithm. These structures were further optimized with a probabilistic optimization algorithm, a variant of simulated annealing, aiming to overcome local energy barriers and follow a kinetically accessible path toward an attainable deep energy minimum with respect to a knowledge-based, protein-oriented potential. This results in 214,142 structures in total for 279 RBD variants. For each structure, the surface accessible surface area (SASA) buried by the interface was calculated. These measurements were aggregated per RBD variant using medians. Each metric is normalized by the metric on wide type, corresponding to no mutation on RBDs, such that the metrics for wide type are all ones.

### Scores Scaling and Merging

The semantic change, log-likelihood, epitope score, ACE2 binding score and growth rate all have different scales and units. Thus, they cannot be compared directly. To make comparisons possible, a scaling strategy is introduced. For a given metric m, all the variants considered are ranked according to this metric. In the ranking system used, the higher rank the better. Variants with the same value for metric m will get the same rank. The ranks are then transformed into values between 0 and 100 through a linear projection to obtain the values for the scaled metric m_{scaled}. All reported scores have been scaled according to this strategy. The immune escape score is computed as the average of the scaled semantic change and of the scaled epitope score. The infectivity score is computed as the sum of the scaled log-likelihood, the scaled ACE2 binding score and the scaled growth rate.

### Pareto Score

Pareto optimality was defined over a set of lineages. Lineages are Pareto optimal within that set if there are no lineages in the set with both higher immune escape and higher infectivity scores. The Pareto score is a measure of the degree of Pareto optimality. Lineages with the highest Pareto score are Pareto optimal. Lineages with the second best Pareto score would be Pareto optimal, if the Pareto optimal lineages were removed from the set, and so on.

### Semantic Change Vs Epitope Alteration Score (EAS)

The number of known nAbs whose binding epitope is affected by a distinct SARS-CoV-2 variants' mutations was defined as the epitope alteration score (EAS).

TABLE 4
Pseudovirus neutralization assays results. The measured reduction
of immune response are reported for a set of selected mutations.

| Lineage | Mutation | |
|---|---|---|
| A.VOI.V2 | D80Y Y144- I210- D215G R246- S247- Y248- L249M W258L R346K T478R E484K | 66.79% |
| | H655Y P681H Q957H | |
| AY.1 | T19R T95I G142D E156G F157- R158- W258L K417N L452R T478K K558N D614G | 48.10% |
| | P681R D950N | |
| B | | 0.00% |
| B.1.1.7 | H69- V70- Y144- N501Y A570D D614G P681H T716I S982A D1118H | 20.72% |
| B.1.1.7 + | H69- V70- Y144- E484K N501Y A570D D614G P681H T716I S982A D1118H | 74.66% |
| E484K | | |
| B.1.160 | S477N S494P D614G K1191N | 3.82% |
| B.1.298 | Y453F D614G I692V M1229I | 5.33% |
| B.1.351 | L18F D80A D215G L242- A243- L244- R246I K417N E484K N501Y D614G A701V | 80.00% |
| B.1.429 | S13I W152C L452R D614G | 52.56% |
| B.1.526 | L5F T95I D253G E484K D614G A701V | 11.43% |
| B.1.617 | L452R E484Q D614G P681R | 28.08% |
| B.1.617.2 | T19R G142D E156G F157- R158- L452R T478K D614G P681R D950N | 49.42% |
| C.37 | G75V T76I R246- S247- Y248- L249- T250- P251- G252-D253N L452Q F490S D614G | 34.23% |
| | T859N | |
| C.37* | G75V T76I L452Q F490S D614G T859N | 16.88% |
| P.1 | L18F T20N P26S D138Y R190S K417T E484K N501Y D614G H655Y T1027I V1176F | 47.67% |

TABLE 5
Early detection of variants of concern. The summary table shows that EWS can detect WHO VUM way before the
WHO official designation date. The average number of days for early detection across the board is 72 days.

| | | | | | | Number of | Number of |
|---|---|---|---|---|---|---|---|
| | | | | | | registered | registered |
| | | EWS | WHO | First | Number of | sequences upon | sequences upon |
| WHO | | Detection | Designation | Submission | Days | WHO | EWS |
| Label | Lineage | Date | Date | Week | Ahead | Detection | Designation |
| Iota | B.1.526 | 2020 Oct. 4 | 2021 Mar. 24 | 2020 Oct. 4 | 171 | 2631 | 1 |
| Alpha | B.1.1.7 | 2020 Oct. 18 | 2020 Dec. 18 | 2020 Oct. 18 | 61 | 1881 | 1 |
| Epsilon | B.1.429 | 2020 Nov. 22 | 2021 Mar. 5 | 2020 Nov. 22 | 103 | 10094 | 2 |
| Beta | B.1.351 | 2020 Nov. 29 | 2020 Dec. 18 | 2020 Nov. 29 | 19 | 1 | 1 |
| Zeta | P.2 | 2020 Dec. 6 | 2021 Mar. 17 | 2020 Dec. 6 | 101 | 1413 | 3 |
| Gamma | P.1 | 2021 Jan. 10 | 2021 Jan. 11 | 2021 Jan. 10 | 1 | 29 | 4 |
| Eta | B.1.525 | 2021 Jan. 10 | 2021 Apr. 4 | 2021 Jan. 10 | 84 | 1184 | 2 |
| Lambda | C.37 | 2021 Feb. 21 | 2021 May 14 | 2020 Nov. 1 | 82 | 758 | 3 |
| Theta | P.3 | 2021 Feb. 28 | 2021 Mar. 24 | 2021 Feb. 28 | 24 | 100 | 1 |
| Mu | B.1.621 | 2021 Mar. 14 | 2021 Aug. 30 | 2021 Mar. 14 | 169 | 4084 | 23 |
| Kappa | B.1.617.1 | 2021 Apr. 4 | 2021 Apr. 4 | 2021 Mar. 7 | 0 | 167 | 167 |
| Delta | B.1.617.2 | / | 2021 Apr. 4 | 2021 Mar. 28 | / | 5 | / |

TABLE 6
N = 310 nAbs Resolved 3D Structures PDB Identifiers

| | Accession | | Accession | | Accession | | Accession | | Accession |
|---|---|---|---|---|---|---|---|---|---|
| Code | Date | Code | Date | Code | Date | Code | Date | Code | Date |
| 6W41 | 2020 Mar. 9 | 7MMO | 2021 Apr. 30 | 7KXJ | 2020 Dec. 4 | 7E5Y | 2021 Feb. 21 | 7CH4 | 2020 Jul. 5 |
| 6WPS | 2020 Apr. 27 | 7MY2 | 2021 May 20 | 7KXK | 2020 Dec. 4 | 7E7X | 2021 Feb. 28 | 7CH5 | 2020 Jul. 5 |
| 6WPT | 2020 Apr. 27 | 7MY3 | 2021 May 20 | 7KZB | 2020 Dec. 10 | 7E7Y | 2021 Feb. 28 | 7CHB | 2020 Jul. 5 |
| 6XC2 | 2020 Jun. 8 | 7N0G | 2021 May 25 | 7L02 | 2020 Dec. 10 | 7E+086 | 2021 Mar. 1 | 7CHC | 2020 Jul. 5 |
| 6XC3 | 2020 Jun. 8 | 7N0H | 2021 May 25 | 7L06 | 2020 Dec. 11 | 7E+088 | 2021 Mar. 1 | 7CHE | 2020 Jul. 5 |
| 6XC4 | 2020 Jun. 8 | 7N0I | 2021 May 25 | 7L09 | 2020 Dec. 11 | 7E8C | 2021 Mar. 1 | 7CHF | 2020 Jul. 5 |
| 6XC7 | 2020 Jun. 8 | 7N0R | 2021 May 25 | 7L0N | 2020 Dec. 11 | 7E8F | 2021 Mar. 1 | 7CHH | 2020 Jul. 5 |
| 6XCM | 2020 Jun. 8 | 7N3C | 2021 May 31 | 7L2C | 2020 Dec. 16 | 7E8M | 2021 Mar. 2 | 7CHO | 2020 Jul. 6 |
| 6XCN | 2020 Jun. 8 | 7N3D | 2021 May 31 | 7L2D | 2020 Dec. 16 | 7EAM | 2021 Mar. 7 | 7CHP | 2020 Jul. 6 |
| 6XDG | 2020 Jun. 10 | 7N3I | 2021 Jun. 1 | 7L2E | 2020 Dec. 16 | 7EAN | 2021 Mar. 7 | 7CHS | 2020 Jul. 6 |
| 6XE1 | 2020 Jun. 11 | 7N62 | 2021 Jun. 7 | 7L2F | 2020 Dec. 16 | 7EJ4 | 2021 Apr. 1 | 7CJF | 2020 Jul. 10 |
| 6XEY | 2020 Jun. 14 | 7N64 | 2021 Jun. 7 | 7L3N | 2020 Dec. 18 | 7EJ5 | 2021 Apr. 1 | 7CM4 | 2020 Jul. 24 |
| 6XKP | 2020 Jun. 26 | 7N8H | 2021 Jun. 14 | 7L56 | 2020 Dec. 21 | 7F62 | 2021 Jun. 24 | 7CR5 | 2020 Aug. 12 |
| 6XKQ | 2020 Jun. 26 | 7N8I | 2021 Jun. 14 | 7L57 | 2020 Dec. 21 | 7F63 | 2021 Jun. 24 | 7CWL | 2020 Aug. 29 |
| 6YLA | 2020 Apr. 6 | 7N9A | 2021 Jun. 17 | 7L58 | 2020 Dec. 21 | 7JMO | 2020 Aug. 2 | 7CWM | 2020 Aug. 29 |
| 6YM0 | 2020 Apr. 7 | 7N9B | 2021 Jun. 17 | 7L5B | 2020 Dec. 21 | 7JMP | 2020 Aug. 2 | 7CWN | 2020 Aug. 29 |
| 6YZ5 | 2020 May 6 | 7N9C | 2021 Jun. 17 | 7LAA | 2021 Jan. 6 | 7JMW | 2020 Aug. 3 | 7CWO | 2020 Aug. 29 |
| 6YZ7 | 2020 May 6 | 7N9E | 2021 Jun. 17 | 7LAB | 2021 Jan. 6 | 7JV2 | 2020 Aug. 20 | 7CWS | 2020 Aug. 31 |
| 6Z2M | 2020 May 17 | 7N9T | 2021 Jun. 18 | 7LCN | 2021 Jan. 11 | 7JV4 | 2020 Aug. 20 | 7CWT | 2020 Aug. 31 |
| 6Z43 | 2020 May 22 | 7ND4 | 2021 Jan. 30 | 7LD1 | 2021 Jan. 12 | 7JV6 | 2020 Aug. 20 | 7CWU | 2020 Aug. 31 |
| 6ZBP | 2020 Jun. 8 | 7ND5 | 2021 Jan. 30 | 7LDJ | 2021 Jan. 13 | 7JVA | 2020 Aug. 20 | 7CYH | 2020 Sep. 3 |
| 6ZCZ | 2020 Jun. 12 | 7ND6 | 2021 Jan. 30 | 7LJR | 2021 Jan. 30 | 7JVB | 2020 Aug. 20 | 7CYP | 2020 Sep. 4 |
| 6ZDG | 2020 Jun. 14 | 7ND7 | 2021 Jan. 30 | 7LM8 | 2021 Feb. 5 | 7JVC | 2020 Aug. 20 | 7CZP | 2020 Sep. 9 |
| 6ZDH | 2020 Jun. 14 | 7ND8 | 2021 Jan. 30 | 7LOP | 2021 Feb. 10 | 7JW0 | 2020 Aug. 24 | 7CZQ | 2020 Sep. 9 |
| 6ZER | 2020 Jun. 16 | 7ND9 | 2021 Jan. 30 | 7LQV | 2021 Feb. 15 | 7JX3 | 2020 Aug. 26 | 7CZR | 2020 Sep. 9 |
| 6ZFO | 2020 Jun. 17 | 7NDA | 2021 Jan. 30 | 7LQW | 2021 Feb. 15 | 7K43 | 2020 Sep. 14 | 7CZS | 2020 Sep. 9 |
| 6ZH9 | 2020 Jun. 21 | 7NDB | 2021 Jan. 30 | 7LRS | 2021 Feb. 17 | 7K45 | 2020 Sep. 14 | 7CZT | 2020 Sep. 9 |
| 6ZHD | 2020 Jun. 22 | 7NDC | 2021 Jan. 30 | 7LRT | 2021 Feb. 17 | 7K4N | 2020 Sep. 15 | 7CZU | 2020 Sep. 9 |
| 6ZLR | 2020 Jul. 1 | 7NDD | 2021 Jan. 30 | 7LS9 | 2021 Feb. 17 | 7K8M | 2020 Sep. 27 | 7CZV | 2020 Sep. 9 |
| 6ZXN | 2020 Jul. 30 | 7NEG | 2021 Feb. 4 | 7LSS | 2021 Feb. 18 | 7K8S | 2020 Sep. 27 | 7CZW | 2020 Sep. 9 |
| 7A25 | 2020 Aug. 16 | 7NEH | 2021 Feb. 4 | 7LX5 | 2021 Mar. 3 | 7K8T | 2020 Sep. 27 | 7CZX | 2020 Sep. 9 |
| 7A29 | 2020 Aug. 16 | 7NKT | 2021 Feb. 18 | 7LXW | 2021 Mar. 5 | 7K8U | 2020 Sep. 27 | 7CZY | 2020 Sep. 9 |
| 7A5R | 2020 Aug. 21 | 7NTC | 2021 Mar. 9 | 7LXX | 2021 Mar. 5 | 7K8V | 2020 Sep. 27 | 7CZZ | 2020 Sep. 9 |
| 7A5S | 2020 Aug. 21 | 7NX6 | 2021 Mar. 17 | 7LXY | 2021 Mar. 5 | 7K8W | 2020 Sep. 27 | 7D00 | 2020 Sep. 9 |
| 7AKD | 2020 Sep. 30 | 7NX7 | 2021 Mar. 17 | 7LXZ | 2021 Mar. 5 | 7K8X | 2020 Sep. 27 | 7D03 | 2020 Sep. 9 |
| 7B14 | 2020 Nov. 23 | 7NX8 | 2021 Mar. 17 | 7LY0 | 2021 Mar. 5 | 7K8Y | 2020 Sep. 27 | 7D0B | 2020 Sep. 9 |
| 7B17 | 2020 Nov. 23 | 7NX9 | 2021 Mar. 17 | 7LY2 | 2021 Mar. 5 | 7K8Z | 2020 Sep. 27 | 7D0C | 2020 Sep. 9 |
| 7B18 | 2020 Nov. 24 | 7NXA | 2021 Mar. 17 | 7LY3 | 2021 Mar. 5 | 7K90 | 2020 Sep. 27 | 7D0D | 2020 Sep. 9 |
| 7B27 | 2020 Nov. 26 | 7NXB | 2021 Mar. 17 | 7M3I | 2021 Mar. 18 | 7K9Z | 2020 Sep. 29 | 7D2Z | 2020 Sep. 17 |
| 7B30 | 2020 Dec. 1 | 7OAN | 2021 Apr. 19 | 7M42 | 2021 Mar. 19 | 7KFV | 2020 Oct. 15 | 7D30 | 2020 Sep. 17 |
| 7BEH | 2020 Dec. 23 | 7OAO | 2021 Apr. 19 | 7M6D | 2021 Mar. 25 | 7KFW | 2020 Oct. 15 | 7D4G | 2020 Sep. 23 |
| 7BEI | 2020 Dec. 23 | 7OAP | 2021 Apr. 19 | 7M6E | 2021 Mar. 25 | 7KFX | 2020 Oct. 15 | 7DCC | 2020 Oct. 24 |
| 7BEJ | 2020 Dec. 23 | 7OAQ | 2021 Apr. 20 | 7M6F | 2021 Mar. 25 | 7KFY | 2020 Oct. 15 | 7DCX | 2020 Oct. 27 |
| 7BEK | 2020 Dec. 23 | 7OAU | 2021 Apr. 20 | 7M6G | 2021 Mar. 25 | 7KGJ | 2020 Oct. 16 | 7DD2 | 2020 Oct. 27 |
| 7BEL | 2020 Dec. 23 | 7OAY | 2021 Apr. 20 | 7M6H | 2021 Mar. 25 | 7KGK | 2020 Oct. 16 | 7DD8 | 2020 Oct. 28 |
| 7BEM | 2020 Dec. 24 | 7OLZ | 2021 May 20 | 7M6I | 2021 Mar. 25 | 7KKK | 2020 Oct. 27 | 7DEO | 2020 Nov. 4 |
| 7BEN | 2020 Dec. 24 | 7OR9 | 2021 Jun. 4 | 7M71 | 2021 Mar. 26 | 7KKL | 2020 Oct. 27 | 7DET | 2020 Nov. 5 |
| 7BEO | 2020 Dec. 24 | 7ORA | 2021 Jun. 4 | 7M7B | 2021 Mar. 27 | 7KLG | 2020 Oct. 30 | 7DEU | 2020 Nov. 5 |
| 7BEP | 2020 Dec. 24 | 7ORB | 2021 Jun. 4 | 7M7W | 2021 Mar. 29 | 7KLH | 2020 Oct. 30 | 7DJZ | 2020 Nov. 22 |
| 7BWJ | 2020 Apr. 14 | 7P77 | 2021 Jul. 19 | 7M8J | 2021 Mar. 29 | 7KLW | 2020 Nov. 1 | 7DK0 | 2020 Nov. 22 |
| 7BYR | 2020 Apr. 24 | 7P78 | 2021 Jul. 19 | 7MDW | 2021 Apr. 6 | 7KM5 | 2020 Nov. 2 | 7DK4 | 2020 Nov. 23 |
| 7BZ5 | 2020 Apr. 26 | 7P79 | 2021 Jul. 19 | 7ME7 | 2021 Apr. 6 | 7KMG | 2020 Nov. 2 | 7DK5 | 2020 Nov. 23 |
| 7C01 | 2020 Apr. 29 | 7P7A | 2021 Jul. 19 | 7MEJ | 2021 Apr. 6 | 7KMH | 2020 Nov. 2 | 7DK6 | 2020 Nov. 23 |
| 7C2L | 2020 May 8 | 7R6W | 2021 Jun. 23 | 7MF1 | 2021 Apr. 8 | 7KMI | 2020 Nov. 2 | 7DK7 | 2020 Nov. 23 |
| 7C8V | 2020 Jun. 3 | 7R6X | 2021 Jun. 23 | 7MFU | 2021 Apr. 11 | 7KMK | 2020 Nov. 3 | 7DPM | 2020 Dec. 20 |
| 7C8W | 2020 Jun. 3 | 7R7N | 2021 Jun. 25 | 7MJI | 2021 Apr. 20 | 7KML | 2020 Nov. 3 | 7DZX | 2021 Jan. 26 |
| 7CAC | 2020 Jun. 8 | 7R8L | 2021 Jun. 26 | 7MJJ | 2021 Apr. 20 | 7KN5 | 2020 Nov. 4 | 7DZY | 2021 Jan. 26 |
| 7CAH | 2020 Jun. 8 | 7R8M | 2021 Jun. 26 | 7MJK | 2021 Apr. 20 | 7KN6 | 2020 Nov. 4 | 7E+023 | 2021 Feb. 4 |
| 7CAI | 2020 Jun. 8 | 7R8N | 2021 Jun. 26 | 7MJL | 2021 Apr. 20 | 7KN7 | 2020 Nov. 4 | 7KSG | 2020 Nov. 22 |
| 7CAK | 2020 Jun. 8 | 7R8O | 2021 Jun. 26 | 7MKL | 2021 Apr. 24 | 7KQB | 2020 Nov. 14 | 7MM0 | 2021 Apr. 29 |
| 7CAN | 2020 Jun. 9 | 7R98 | 2021 Jun. 28 | 7MKM | 2021 Apr. 24 | 7KQE | 2020 Nov. 15 | 7RAL | 2021 Jul. 1 |
| 7CDI | 2020 Jun. 19 | 7RA8 | 2021 Jun. 30 | 7MLZ | 2021 Apr. 29 | 7KS9 | 2020 Nov. 21 | 7CDJ | 2020 Jun. 19 |

TABLE 7
Welch's T-test p-values. Every week, all registered variants
are scored with the Pareto score. Welch's t-tests are conducted
to assess if respectively designated variants and VOCs can
be separated from others. p-values are reported every week.

Welch t-test p-values

| | WHO | |
|---|---|---|
| | Designated | |
| Week | Variants | VOC |
| 2020 Dec. 13 | 8E−118 | 8E−118 |
| 2020 Dec. 20 | 7E−07 | 7E−07 |
| 2020 Dec. 27 | 5E−128 | 5E−128 |
| 2021 Jan. 3 | 7E−06 | 7E−06 |
| 2021 Jan. 10 | 5E−140 | 5E−140 |
| 2021 Jan. 17 | 2E−07 | 2E−07 |
| 2021 Jan. 24 | 4E−06 | 4E−06 |
| 2021 Jan. 31 | 5E−144 | 5E−144 |
| 2021 Feb. 7 | 2E−06 | 2E−06 |
| 2021 Feb. 14 | 6E−28 | 4E−19 |
| 2021 Feb. 21 | 7E−21 | 9E−08 |
| 2021 Feb. 28 | 1E−16 | 2E−08 |
| 2021 Mar. 7 | 8E−07 | 2E−19 |
| 2021 Mar. 14 | 2E−05 | 2E−07 |
| 2021 Mar. 21 | 1E−05 | 1E−20 |
| 2021 Mar. 28 | 1E−05 | 1E−22 |
| 2021 Apr. 4 | 4E−06 | 2E−19 |
| 2021 Apr. 11 | 3E−05 | 4E−18 |
| 2021 Apr. 18 | 5E−06 | 2E−20 |
| 2021 Apr. 25 | 6E−06 | 4E−08 |
| 2021 May 2 | 2E−12 | 5E−07 |
| 2021 May 9 | 3E−08 | 5E−06 |
| 2021 May 16 | 4E−09 | 8E−06 |
| 2021 May 23 | 1E−06 | 4E−04 |
| 2021 May 30 | 6E−07 | 4E−04 |
| 2021 Jun. 6 | 2E−04 | 2E−04 |
| 2021 Jun. 13 | 4E−05 | 2E−05 |
| 2021 Jun. 20 | 8E−06 | 2E−04 |
| 2021 Jun. 27 | 3E−05 | 3E−05 |
| 2021 Jul. 4 | 1E−04 | 2E−03 |
| 2021 Jul. 11 | 1E−05 | 1E−05 |
| 2021 Jul. 18 | 1E−04 | 3E−04 |
| 2021 Jul. 25 | 1E−03 | 1E−07 |
| 2021 Aug. 1 | 2E−04 | 6E−09 |
| 2021 Aug. 8 | 7E−06 | 5E−09 |
| 2021 Aug. 15 | 2E−05 | 9E−06 |
| 2021 Aug. 22 | 4E−07 | 3E−21 |
| 2021 Aug. 29 | 3E−05 | 4E−05 |
| 2021 Sep. 5 | 1E−04 | 5E−04 |
| 2021 Sep. 12 | 5E−05 | 1E−04 |
| 2021 Sep. 19 | 1E−04 | 2E−04 |
| 2021 Sep. 26 | 2E−06 | 9E−06 |
| 2021 Oct. 3 | 1E−04 | 1E−05 |
| 2021 Oct. 10 | 2E−06 | 7E−06 |
| 2021 Oct. 17 | 1E−07 | 4E−07 |
| 2021 Oct. 24 | 7E−07 | 3E−08 |
| 2021 Oct. 31 | 2E−05 | 2E−05 |
| 2021 Nov. 7 | 2E−04 | 3E−04 |
| 2021 Nov. 14 | 4E−03 | 3E−04 |
| 2021 Nov. 21 | 5E−05 | 7E−06 |

### Example 2: Exemplary Variants and their Categorization as Assigned by the European Centre for Disease Prevention and Control

The European Centre for Disease Prevention and Control (ECDPC) maintains a web site (www.ecdc.europa.eu/en/covid-19/variants-concern that includes tables listing “Variants of Concern” (VOC), “Variants of Interest” (VOI) or Variants Under Monitoring (VUM); similar information is provided by the World Health Organization (see www.who.int/en/activities/tracking-SARS-CoV-2-variants/). Both web sites provide information like the Country in which the listed variant was first detected, and certain (but not all) of the sequence changes identified in that variant's spike protein.

The EPDPC web site indicates that its Tables include at least those spike protein changes that are between residues 319-541 (receptor binding domain) or 613-705 (the S1 portion of the S1/S2 junction plus some sequences on the S2 side), as well as “additional unusual changes” specific to the variant. Additional lineage information for each variant can be found at cov-lineages.org/lineage_list.html.

As of Nov. 26, 2021, the listed Variants of Concern were as indicated below in Table 8:

TABLE 8
Variants of Concerns as of Nov. 26, 2021

| | | | | | | Evidence | Evidence | |
|---|---|---|---|---|---|---|---|---|
| | Lineage + | Country first | Spike | Year and | Evidence for | for | for | |
| WHO | additional | detected | mutations | month first | impact on | impact | impact | Transmission |
| label | mutations | (community) | of interest | detected | transmissibility | on immunity | on severity | in EU/EEA |
| Beta | B.1.351 | South | K417N, | September | Yes (v) | Yes (v) | Yes (v) | Community |
| | | Africa | E484K, | 2020 | (1) | (2, 3) | (4, 5) | |
| | | | N501Y, | | | | | |
| | | | D614G, | | | | | |
| | | | A701V | | | | | |
| Gamma | P.1 | Brazil | K417T, | December | Yes (v) | Yes (v) | Yes (v) | Community |
| | | | E484K, | 2020 | (6) | (7) | (5) | |
| | | | N501Y, | | | | | |
| | | | D614G, | | | | | |
| | | | H655Y | | | | | |
| Delta | B.1.617.2 | India | L452R, | December | Yes (v) | Yes (v) | Yes (v) | Dominant |
| | | | T478K, | 2020 | (8) | (9-11) | (10, 12) | |
| | | | D614G, | | | | | |
| | | | P681R | | | | | |
| Omicron | B.1.1.529 | South | (x) | November | | | | Sporadic/ |
| | | Africa and | | 2021 | | | | Travel |
| | | Botswana | | | | | | |

(x): A67V, Δ69-70, T95I, G142D, Δ143-145, Δ211-212, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, L981F
All sub-lineages of the listed lineages are also included in the variant, e.g., C.37.1 is included in Lambda as it is a sub-lineage of C.37.

The present disclosure, in addition to successfully and rapidly identifying characteristics of, for example, the beta and delta variants (see herein), furthermore identifies the omicron variant as within the top 0.005% of immune escaping variants.

For completeness, we note that, as of Nov. 26, 2021, this web site identified the following (shown in Table 9) as current Variants of Interest:

TABLE 9
Variants of Interest as of Nov. 26, 2021

| | | | | Year and | | Evidence | Evidence | |
|---|---|---|---|---|---|---|---|---|
| | Lineage + | Country first | Spike | month | Evidence for | for | for | |
| WHO | additional | detected | mutations | first | impact on | impact on | impact on | Transmission |
| label | mutations | (community) | of interest | detected | transmissibility | immunity | severity | in EU/EEA |
| Mu | B.1.621 | Colombia | R346K, | January | Yes (m) | Yes (m) | | Sporadic/ |
| | | | E484K, | 2021 | (13) | (14) | | Travel |
| | | | N501Y, | | | | | |
| | | | D614G, | | | | | |
| | | | P681H | | | | | |
| Lambda | C.37 | Peru | L452Q, | December | | Yes (v) | | Sporadic/ |
| | | | F490S, | 2020 | | (15, 16) | | Travel |
| | | | D614G | | | | | |
| n/a | AY.4.2 | United | L452R, | June | Yes (v) | No (v) | No (v) | Community |
| | | Kingdom | T478K, | 2021 | (17) | (17, 18) | (17) | |
| | | | D614G, | | | | | |
| | | | P681R, | | | | | |
| | | | A222V, | | | | | |
| | | | Y145H | | | | | |

n/a: not applicable,
no WHO label has been assigned to this variant at this time
All sub-lineages of the listed lineages are also included in the variant, e.g., AZ.1 is included in B.1.1.318 as it is a sub-lineage of it.

Also as of this date, this web site identified the following (shown in Table 10) as current Variants under Monitoring:

TABLE 10
Variants under Monitoring as of Nov. 26, 2021

| | | | | | | | | |
|---|---|---|---|---|---|---|---|---|
| | | | | | | Evidence | Evidence | |
| | Lineage + | Country first | Spike | Year and | Evidence for | for | for | |
| WHO | additional | detected | mutations of | month first | impact on | impact on | impact on | Transmission |
| label | mutations | (community) | interest | detected | transmissibility | immunity | severity | in EU/EEA |

| | | | | | | | |
|---|---|---|---|---|---|---|---|
| n/a | C.36 + L452R | Egypt | L452R, | December | | Yes (m) | Detected |
| | | | D614G, | 2020 | | (19) | (a) |
| | | | Q677H | | | | |
| n/a | B.1.1.318 | Unclear | E484K, | January | | Yes (m) | Detected |
| | | (b) | D614G, | 2021 | | (14) | (a) |
| | | | P681H | | | | |
| n/a | P.1 + P681H | Italy | D614G, | February | | Unclear | Detected |
| | | | E484K, | 2021 | | (20, 21) | (a) |
| | | | H655Y, | | | | |
| | | | K417T, | | | | |
| | | | N501Y, | | | | |
| | | | P681H | | | | |
| n/a | B.1.617.2 + | United | L452R, | June | | | Detected |
| | K417N | Kingdom | T478K, | 2021 | | | (a) |
| | | | D614G, | | | | |
| | | | P681R, | | | | |
| | | | K417N | | | | |
| n/a | C.1.2 | South | D614G, | June | Yes (m) | Yes (m) | Detected |
| | | Africa | E484K, | 2021 | (13) | (14) | (a) |
| | | | H655Y, | | | | |
| | | | N501Y, | | | | |
| | | | N679K, | | | | |
| | | | Y449H | | | | |
| n/a | B.1.617.2 + | India | L452R, | April | | | Detected |
| | E484X (d) | | T478K, | 2021 | | | (a) |
| | | | D614G, | | | | |
| | | | P681R, | | | | |
| | | | E484X (d) | | | | |
| n/a | B.1.617.2 + | India | L452R, | April | | | Detected |
| | Q613H | | T478K, | 2021 | | | (a) |
| | | | D614G, | | | | |
| | | | P681R, | | | | |
| | | | Q613H | | | | |
| n/a | B.1.617.2 + | India | L452R, | April | | | Detected |
| | Q677H | | T478K, | 2021 | | | (a) |
| | | | D614G, | | | | |
| | | | P681R, | | | | |
| | | | Q677H | | | | |
| n/a | B.1.640 | The | D614G, | September | | | Detected |
| | | Republic | F490R, | 2021 | | | (a) |
| | | of Congo | N394S, | | | | |
| | | | N501Y, | | | | |
| | | | P681H, | | | | |
| | | | R346S, | | | | |
| | | | Y449N, | | | | |
| | | | 137-145del | | | | |

n/a: not applicable,
no WHO label has been assigned to this variant at this time
All sub-lineages of the listed lineages are also included in the variant, e.g., AZ.1 is included in B.1.1.318 as it is a sub-lineage of it.

### Example 3: Exemplary Modifications to Methods and Systems Described Herein Including EWS

In some embodiments, the EWS described in Example 1 can be adjusted to further improve its predictive abilities and/or better accommodate the viral variants being assessed.

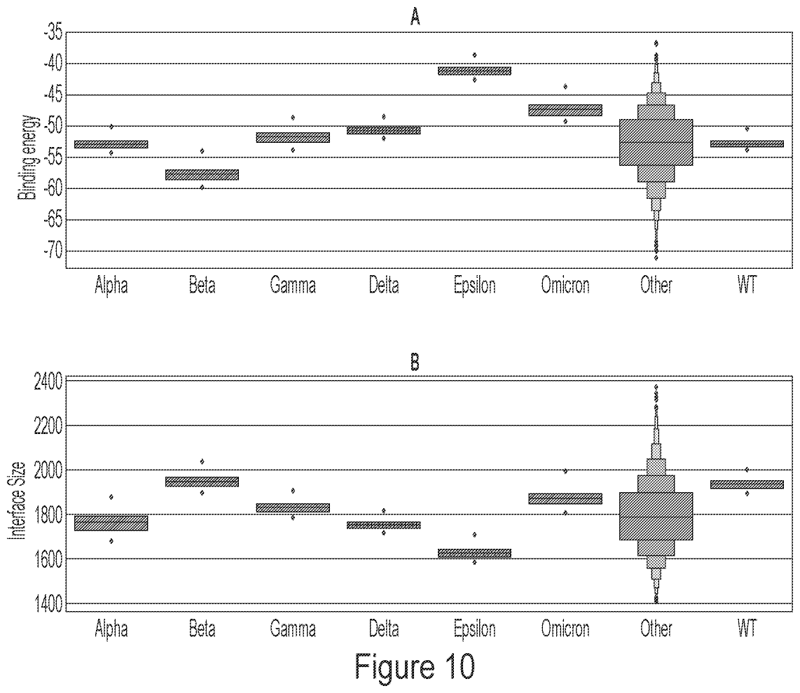

In some embodiments, the EWS was adjusted so as to calculate the ACE2 binding score using the difference in Gibbs free energy between the bound and unbound structures of ACE2 and the RBD (results shown in FIG. 10). 279 receptor-binding domain (RBD) differentiated variants, including the wide type, were simulated in-silico. For each variant, a putative structure was generated, from which at least 500 structures were generated through a conformational sampling algorithm. These structures were then further optimized with a probabilistic optimization algorithm, a variant of simulated annealing, aiming to overcome local energy barriers and follow a kinetically accessible path toward an attainable deep energy minimum with respect to a knowledge-based, protein-oriented potential. This resulted in 214,142 structures in total. For each structure, the change of binding energy when the interface forming chains are separated was then calculated, as compared to when they are complexed. These measurements were aggregated per RBD variant using medians. Each metric was normalized by the metric on relative to the wild type Spike protein (corresponding to no mutation on RBDs), such that the metrics for the wild type sequence are all ones. Sequences having other RBD mutation combinations, representing very rare RBDs, corresponding to <9% of all known sequences, were excluded from this analysis, due to reasons of computational efficiency.

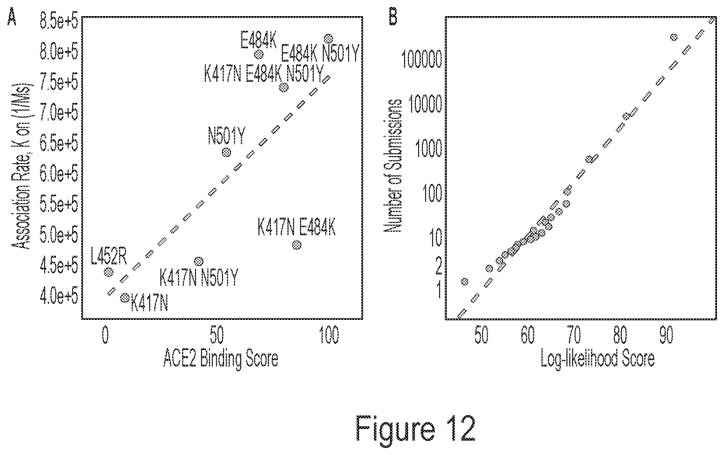

In order to assess the validity of the ACE2 binding score, the simulation results were compared with in vitro results reported by Tanaka et al. (Tanaka et al., 2021). The authors performed a biolayer interferometry (BLI) kinetic analysis to measure the association rate (kₒₙ) for targeted sets of mutations and showed the relationship between RBD mutations and the RBD-ACE2 association rate. ACE2 binding score determined using methods described herein showed meaningful correlation with the association rate (kₒₙ) for targeted sets with a Pearson correlation coefficient of 0.75. (FIG. 12.A).

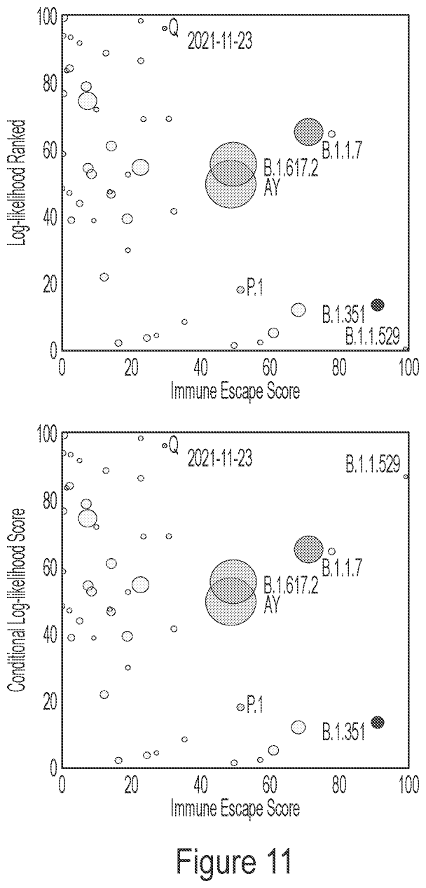

In some embodiments, the log-likelihood score may be adjusted so as to better accommodate variant sequences that have acquired a large number of mutations. Without wishing to be bound by any theory, for some viral variants, log-likelihood values tend to diminish with the increasing number of mutations, given the definition of this metric; it over-emphasizes variants with low mutation counts. Considering that all the samples used for training have been detected in patients, and as such have likely satisfied a minimal fitness criteria, a conditional log-likelihood score can be introduced so as to measure how the log-likelihood of the variant in question compares to other variants with similar mutational loads, as opposed to the entire population. The observed relationship of log-likelihood to expected sequence is predictive count holds as well for the log-likelihood score (FIG. 12B), as most of the circulating sequences have low mutation counts, making both distributions very similar. This metric can shed more light on highly mutated, potentially concerning variants like B.1.1.529 (Omicron). Due to its expected frequency in population and high mutational load, this variant might be perceived by raw log-likelihood as highly unlikely. However, relative to other variant sequences with a similar number of mutations, it becomes clear that it stands out, leading to a high log-likelihood score (FIG. 11).

A variant with two mutations, whose log-likelihood is in the bottom 20th percentile globally, is less likely to survive the evolutionary competition. A variant, with analogous log-likelihood, but with twenty mutations in contrast, is more likely to survive the evolutionary competition than other, similarly mutated variants. Thus, a group-based ranking strategy was introduced, where each variant was ranked only among variants that have a similar number of mutations. For each variant, having N mutations, its log-likelihood score was ranked among all variants having at least M mutations, with M=min(max(0, N−10), 50). Deletions at N-terminal or C-terminal were considered as one single mutation for grouping. In each group, as for the other ranking technique, the ranks were then transformed into values between 0 and 100 through a linear projection to obtain the values for the scaled metric. Although this approach compares all samples having no less than ten mutations fewer than the query, the results were found to be largely robust to the choice of a threshold.

In some embodiments, the semantic change of a variant x was be computed as:

$$Δ \overline{z} = {‖ \overline{z} - {\overline{z}}_{wuhuan} ‖}_{1} + {‖ \overline{z} - {\overline{z}}_{D 614 G} ‖}_{1} ,$$

where ∥⋅∥₁ is the L1 norm in the updated EWS.

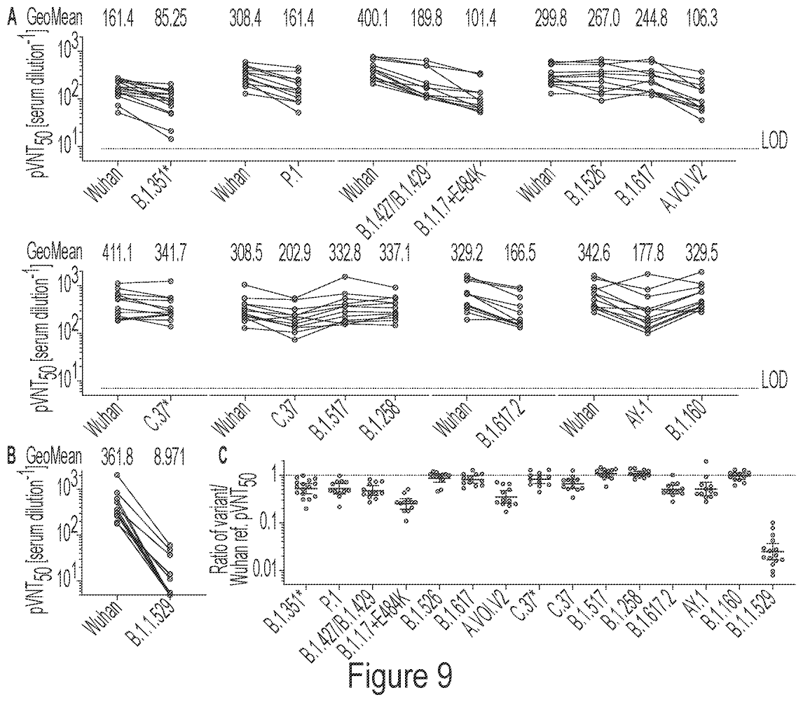

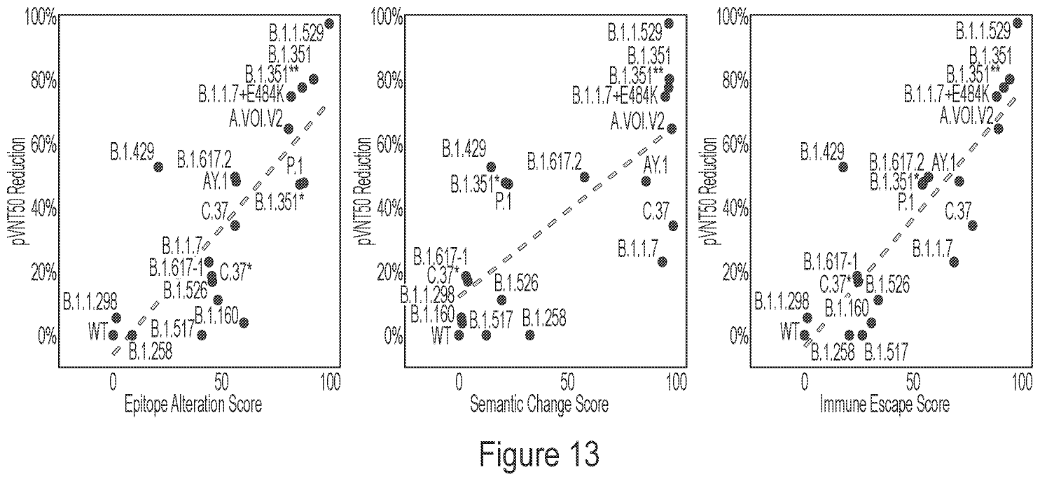

Immune escape in silico metrics were determined as described in Example 1. In vitro pseudovirus neutralization test (pVNT) assay results were used to validate the immune escape in silico metrics: semantic change and epitope alteration score. The cross-neutralizing effect of n≥12 BNT162b2-immune sera was assessed against vesicular stomatitis virus (VSV)-SARS-CoV-2-S pseudoviruses bearing the spike protein of n=19 selected High Risk Variants, including Omicron (B.1.1.529) (FIG. 13, FIG. 9, Table 11) (Muik et al., 2021; Sahin et al., 2021). The SARS-CoV-2 Omicron pseudovirus was by far the most immune escaping with >20-fold reduction of the 50% pseudovirus neutralisation titer (pVNT₅₀) compared with the geometric mean titer (GMT) against the Wuhan reference spike-pseudotyped VSV (FIG. 13). The calculated geometric mean ratio with 95% confidence interval (CI) of the Omicron pseudotype and the Wuhan pseudotype GMTs was 0.025 (95% CI; 0.017 to 0.037), indicating another 10-fold drop of the neutralising activity against Omicron compared to the second most immune escaping B.1.1.7+E484K pseudovirus with a geometric mean ratio of 0.253 (95% CI; 0.196 to 0.328) (FIG. 9C). This result is in concordance with the in silico immune escape score for Omicron, which is the highest amongst observed, circulating variants. Across all HRV pseudoviruses tested, both the epitope alteration score and the semantic change score calculated using the updated EWS were found to correlate positively with the calculated 50% pseudovirus neutralization titer (pVNT50) reduction (FIG. 13; Pearson r=0.73 and 0.71, respectively). An average of both in silico scores (summarized as the ‘immune escape score’) was again found to exhibit a slightly stronger correlation with the observed reduction in neutralizing titers (Pearson r=0.81).

TABLE 11
Spike mutations in SARS-CoV-2 Spike pseudoviruses and observed reduction
of neutralizing antibody response in pseudovirus neutralization assay.

| Lineage | WHO Nomenclature | Mutation | Reduction |
|---|---|---|---|
| B.1.1.7 | Alpha | H69- V70- Y144- N501Y A570D D614G P681H T716I | 22.92% |
| | | S982A D1118H | |
| B.1.1.7 + | Alpha | H69- V70- Y144- E484K N501Y A570D D614G P681H | 74.65% |
| E484K | | T716I S982A D1118H | |
| B.1.351 | Beta | L18F D80A D215G L242- A243- L244- R246I K417N | 80.04% |
| | | E484K N501Y D614G A701V | |
| B.1.351* | Beta | D80A D215G L242H K417N E484K N501Y D614G A701V | 47.19% |
| B.1.351** | Beta | D80A D215G L242- A243- L244- K417N E484K N501Y | 77.45% |
| | | D614G A701V | |
| P.1 | Gamma | L18F T20N P26S D138Y R190S K417T E484K N501Y | 47.66% |
| | | H655Y T1027I V1176F | |
| B.1.617.2 | Delta | T19R G142D E156G F157- R158- K417N L452R T478K | 49.43% |
| | | D614G P681R D950N | |
| AY.1 | Delta | T19R T95I G142D E156G F157- R158- W258L K417N | 48.09% |
| | | L452R T478K K558N D614G P681R D950N | |
| B.1.427/ | Epsilon | S13I W152C L452R D614G | 52.57% |
| B.1.429 | | | |
| B.1.526 | Iota | L5F T95I D253G E484K D614G A701V | 10.94% |
| B.1.617.1 | Kappa | L452R E484Q D614G P681R | 18.34% |
| C.37 | Lambda | G75V T76I R246- S247- Y248- L249- T250- P251- G252- | 34.22% |
| | | D253N L452Q F490S D614G T859N | |
| C.37* | Lambda | G75V T76I L452Q F490S D614G T859N | 16.88% |
| B.1.1.529 | Omicron | A67V H69- V70- T95I G142D V143- Y144- Y145- N211I | 97.52% |
| | | L212V ins214EPE G339D S371L S373P S375F K417N | |
| | | N440K G446S S477N T478K E484A Q493R G496S Q498R | |
| | | N501Y Y505H T547K D614G H655Y N679K P681H | |
| | | N764K D796Y N856K Q954H N969K L981F | |
| A.VOI.V2 | | D80Y Y144- I210- D215G R246- S247- Y248- L249M | 64.54% |
| | | W258L R346K T478R E484K H655Y P681H Q957H | |
| B.1.1.298 | | Y453F D614G I692V M1229I | 5.42% |
| B.1.160 | | S477N S494P D614G K1191N | 3.81% |
| B.1.258 | | H69- V70- L189F N439K D614G V772I | −9.28% |
| B.1.517 | | G181V G252V N501T D614G P812L | −7.87% |

The observed relationship of log-likelihood to expected sequence count holds as well for the log-likelihood score (FIG. 12B), as most of the circulating sequences have low mutation counts, making both distributions very similar. This metric sheds more light on highly mutated, potentially concerning variants like B.1.1.529 (Omicron). Due to its expected frequency in population. high mutational load, this variant might be perceived by raw log-likelihood as highly unlikely. However, relative to other variant sequences with a similar number of mutations, it becomes clear that it stands out, leading to a high log-likelihood score (FIG. 11).

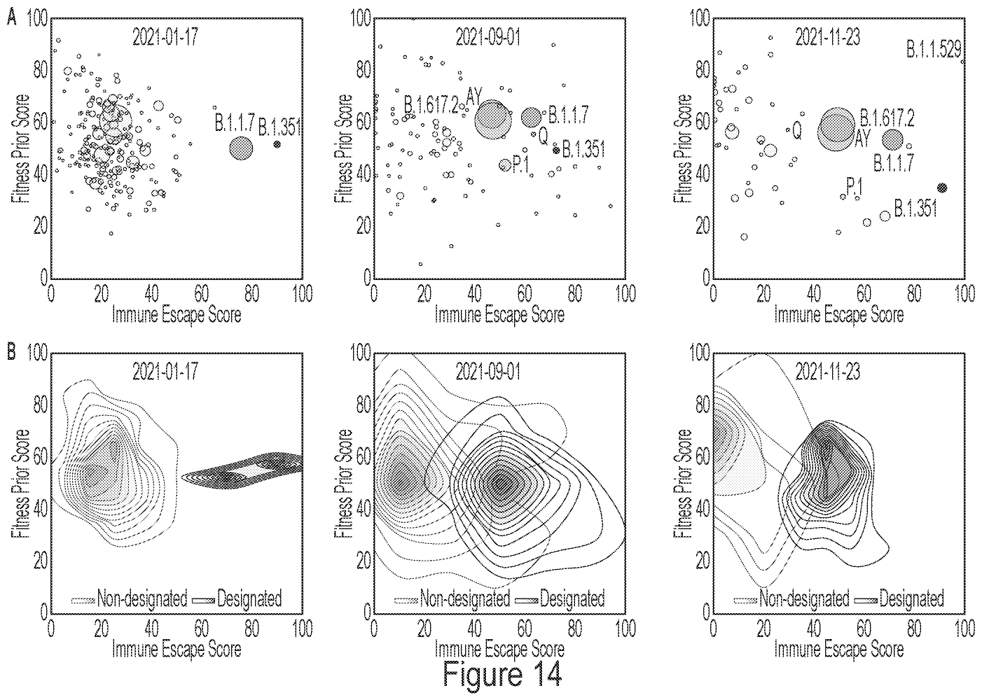

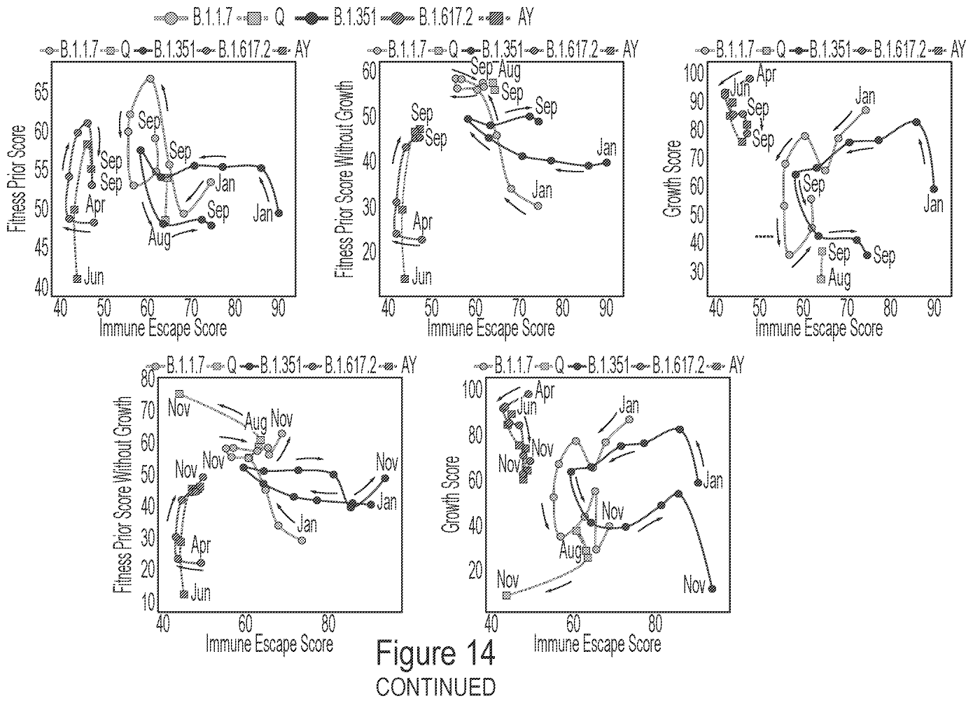

Immune escape scores and fitness prior scores were calculated using the updated EWS (FIGS. 14A to 14C). As seen before, variants of concern were found to start off relatively far into the upper-right corner (i.e. are comparatively highly immune escaping and have satisfactory fitness prior score for their immune escape value).

In order to validate that the Pareto score separates WHO designated variants from non-designated variants, Welch's t-tests were conducted over the registered variants population every week from January 2021 to the end of November 2021 (Table 12). The null hypothesis can be rejected with a p-value<alpha=0.05, for all 50 weeks, thus demonstrating that the Pareto score can separate designated from non-designated variants continuously through time.

TABLE 12
Welch's t-test p-values. Every week, all registered variants
were scored with the Pareto score. Welch's t-tests were
conducted to assess if respectively WHO designated variants
and VOCs can be separated from others. p-values reported
every week between January 2020 and November 2021.

Welch t-test p-values

| | WHO | |
|---|---|---|
| | Designated | |
| Week | Variants | VOC |
| 2021 Jan. 3 | 5E−07 | 5E−07 |
| 2021 Jan. 10 | 7E−144 | 7E−144 |
| 2021 Jan. 17 | 2E−143 | 2E−143 |
| 2021 Jan. 24 | 3E−146 | 3E−146 |
| 2021 Jan. 31 | 5E−159 | 5E−159 |
| 2021 Feb. 7 | 3E−08 | 3E−08 |
| 2021 Feb. 14 | 1E−24 | 2E−23 |
| 2021 Feb. 21 | 3E−23 | 2E−28 |
| 2021 Feb. 28 | 1E−17 | 7E−30 |
| 2021 Mar. 7 | 1E−07 | 3E−25 |
| 2021 Mar. 14 | 4E−07 | 5E−26 |
| 2021 Mar. 21 | 3E−05 | 8E−21 |
| 2021 Mar. 28 | 1E−06 | 3E−25 |
| 2021 Apr. 4 | 5E−07 | 6E−26 |
| 2021 Apr. 11 | 1E−06 | 6E−29 |
| 2021 Apr. 18 | 2E−06 | 1E−28 |
| 2021 Apr. 25 | 5E−06 | 6E−11 |
| 2021 May 2 | 7E−15 | 2E−14 |
| 2021 May 9 | 2E−09 | 3E−06 |
| 2021 May 16 | 5E−12 | 2E−53 |
| 2021 May 23 | 1E−07 | 4E−11 |
| 2021 May 30 | 1E−07 | 7E−07 |
| 2021 Jun. 6 | 4E−06 | 1E−09 |
| 2021 Jun. 13 | 1E−05 | 3E−08 |
| 2021 Jun. 20 | 9E−07 | 1E−05 |
| 2021 Jun. 27 | 4E−04 | 3E−04 |
| 2021 Jul. 4 | 1E−05 | 3E−05 |
| 2021 Jul. 11 | 1E−06 | 2E−05 |
| 2021 Jul. 18 | 1E−05 | 2E−06 |
| 2021 Jul. 25 | 2E−03 | 3E−03 |
| 2021 Aug. 1 | 3E−03 | 3E−04 |
| 2021 Aug. 8 | 6E−04 | 4E−08 |
| 2021 Aug. 15 | 4E−05 | 5E−06 |
| 2021 Aug. 22 | 3E−05 | 2E−09 |
| 2021 Aug. 29 | 2E−03 | 9E−04 |
| 2021 Sep. 5 | 2E−05 | 6E−04 |
| 2021 Sep. 12 | 9E−06 | 5E−04 |
| 2021 Sep. 19 | 7E−07 | 5E−05 |
| 2021 Sep. 26 | 1E−07 | 2E−09 |
| 2021 Oct. 3 | 1E−10 | 3E−06 |
| 2021 Oct. 10 | 3E−14 | 2E−12 |
| 2021 Oct. 17 | 5E−08 | 9E−08 |
| 2021 Oct. 24 | 2E−07 | 2E−05 |
| 2021 Oct. 31 | 7E−10 | 5E−09 |
| 2021 Nov. 7 | 3E−08 | 3E−09 |
| 2021 Nov. 14 | 4E−04 | 7E−07 |
| 2021 Nov. 21 | 4E−04 | 7E−04 |

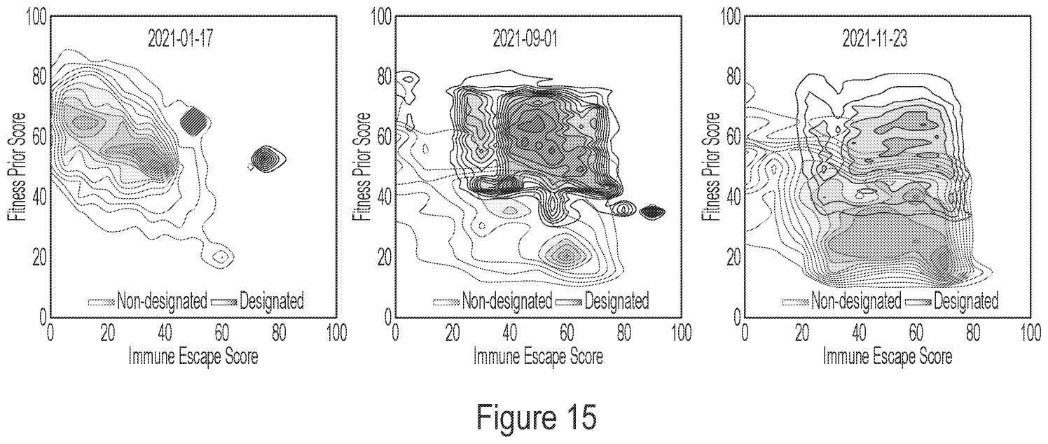

For visualization purposes, a focus was made on three dates of interest: the 17th of January 2021, the 1st of September 2021 and the 23rd of November 2021. At these dates, p-values=2E-143, 6E-4 and 4E-4 were reported. Density contour estimates conducted on Jan. 17, 2021 and, Sep. 1, 2021 and Nov. 23, 2021 also demonstrate clear separability between WHO designated variants and non-designated ones per lineage: FIG. 14B and per sequence: FIG. 15). Importantly, they suggest that immune escape significantly contributes to this separability, more so than the fitness prior score.

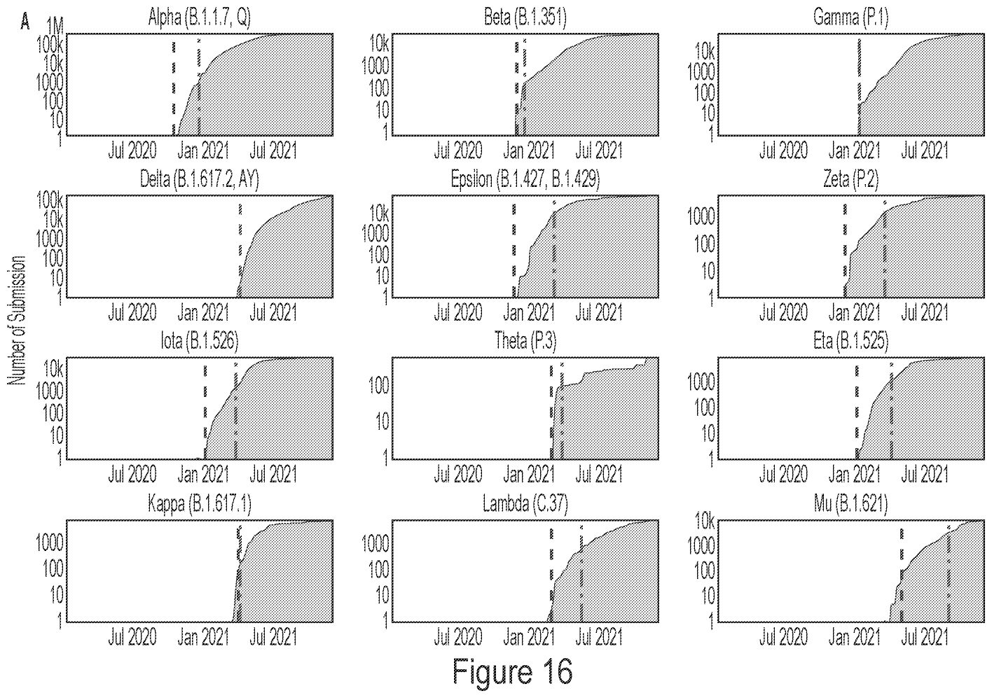

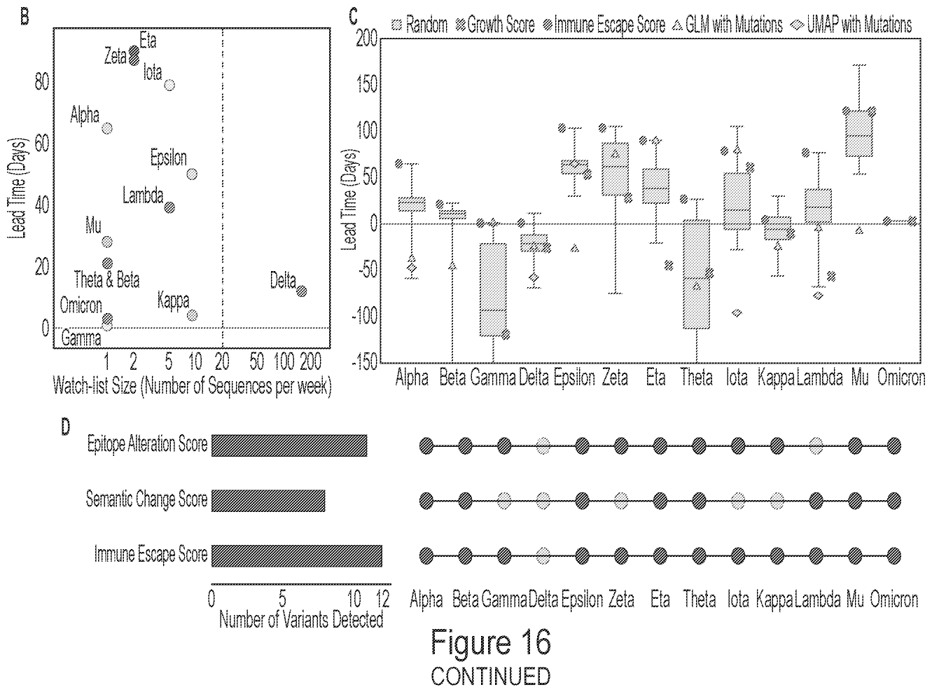

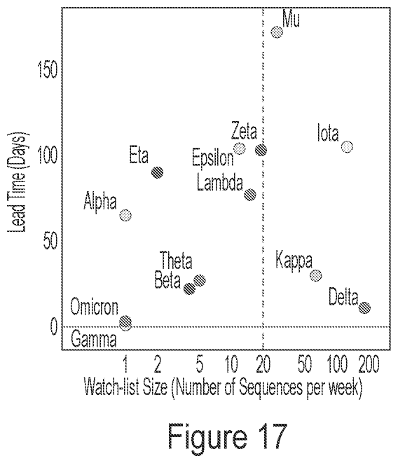

A retrospective analysis was conducted using the updated EWS for every week between Sep. 16, 2020 and Nov. 23, 2021. The EWS ranked all new sequences on immune escape to compile a weekly flagged HRV watch-list. The models were only trained on variants up to the previous month's start date and any other data used were prior to the analysis date. To assess the system's sensitivity, we focused on the detection of the 13 variants designated by WHO (Alpha, Beta, Gamma, Delta, Epsilon, Eta, Zeta, Theta, Iota, Kappa, Lambda, Mu, and Omicron). When using a weekly watch-list with a size of 20 variants (less than 10.5% of the weekly average of new variant sequences), EWS flagged 12 WHO designated variants out of 13 (FIG. 16A), with an average of 58 days of lead time (i.e. two months) before these were designated as such by the WHO (Table 13). For variants Alpha to Mu for which there is now sufficient data, on average only 0.5% of cases of that variant were already recorded at the time of their detection by the EWS (1925 sequences on average), to be contrasted with the WHO announcements that happened on average when 18% of cases for that variant were already recorded (1,593 sequences on average). Different watch-list sizes ranging from 1 up to 200 variants were assessed to evaluate detection sensitivity (FIG. 16B). The number of named variants detected remained stable when varying the weekly watch list size between 10 and 200. While a longer list compromises specificity, it leads to an increase in the detection lead time (the number of days ahead of WHO designation) (FIG. 17).

Strikingly, WHO-designated variants Alpha, Beta, Gamma, Theta, Eta, and Omicron were detected by the EWS on the same week they were first reported, even in the extreme case where the weekly watch-list allowed only one variant, meaning they were the top-scoring sequences among all emerging variants that week (FIG. 16B). Using a larger weekly watch-list of 20 variants, Epsilon, Zeta, and Lambda were also detected in the same week they were first reported, on top of the previously mentioned WHO-designated variants (9 in total detected the first week).

Specifically, the EWS identified Omicron as the highest immune escaping variant over more than 70,000 variants discovered between early October and late November 2021. This variant combines frequent RBD mutations (K417N, S477N, N501Y), with less frequent ones (G339D, S371L, S373P, S375F, Q498R) to potentially evade RBD-targeting antibodies. The NTD indels in positions 69-70, 143-145, 211-214 alter known antibody recognition sites as well. These mutations, together with over 20 others, led to an exceptional epitope alteration score, the highest recorded since the beginning 465 of the pandemic and a high semantic change score, which combined rank Omicron in the top 0.005% of variants on immune escape since the pandemic started.

TABLE 13
Early detection of variants of concern. The summary table shows that the EWS can detect WHO designated
variants months before the WHO official designation date. The average lead time for early detection
across was 58 days. Notably, Omicron was flagged by the EWS on the day its sequence was made available,
with immune evasion and binding metrics subsequence confirmed through in vitro experiments.

| | | | | | | Number of | Number of |
|---|---|---|---|---|---|---|---|
| | | | | | | registered | registered |
| | | EWS | WHO | First | | sequences | sequences |
| | | Detection | Designation | Submission | Lead Time | upon WHO | upon EWS |
| WHO Label | Lineage | Date | Date | Date | (Days) | Detection | Designation |
| Alpha | B.1.1.7 | 2020 Oct. 14 | 2020 Dec. 18 | 2020 Oct. 14 | 65 | 1881 | 1 |
| Beta | B.1.351 | 2020 Nov. 27 | 2020 Dec. 18 | 2020 Nov. 26 | 21 | 218 | 15 |
| Gamma | P.1 | 2021 Jan. 10 | 2021 Jan. 11 | 2021 Jan. 10 | 1 | 29 | 4 |
| Delta | B.1.617.2 | | 2021 Apr. 4 | 2021 Mar. 23 | | 5 | |
| Epsilon | B.1.429 | 2020 Nov. 21 | 2021 Mar. 5 | 2020 Nov. 21 | 104 | 9957 | 1 |
| Zeta | P.2 | 2020 Dec. 4 | 2021 Mar. 17 | 2020 Dec. 2 | 103 | 1450 | 3 |
| Eta | B.1.525 | 2021 Jan. 4 | 2021 Apr. 4 | 2021 Jan. 4 | 90 | 1377 | 6 |
| Theta | P.3 | 2021 Feb. 25 | 2021 Mar. 24 | 2021 Feb. 25 | 27 | 102 | 1 |
| Iota | B.1.526 | 2021 Jan. 4 | 2021 Mar. 24 | 2020 Dec. 9 | 79 | 1120 | 2 |
| Kappa | B.1.617.1 | 2021 Mar. 31 | 2021 Apr. 4 | 2021 Mar. 5 | 4 | 167 | 167 |
| Lambda | C.37 | 2021 Feb. 26 | 2021 May 14 | 2021 Feb. 26 | 77 | 719 | 4 |
| Mu | B.1.621 | 2021 Apr. 30 | 2021 Aug. 30 | 2021 Mar. 11 | 122 | 3671 | 88 |
| Omicron | B.1.1.529 | 2021 Nov. 23 | 2021 Nov. 26 | 2021 Nov. 23 | 3 | 11 | 10 |

The immune escape score used by the EWS to early detect HRVs combines the epitope alteration and semantic change score. The early detection performance of each of these two components separately and combined was evaluated: while the Epitope Alteration Score detects 11 out of 13 WHO designated variants ahead of time, the Semantic Change score detects 8 out of 13. Their combination, however, flagged 12 out of 13 WHO designated variants (FIG. 16D). This validates the approach of associating protein structure modeling and transformer language models on protein sequence to accurately rank SARS-CoV-2 variants.

### Example 4: Exemplary In Vitro Methods for Assessment of Infectivity/Transmissibility Metric of Variants

In some embodiments, infectivity/transmissibility metric of variants were assessed using surface plasmon resonance spectroscopy to assess binding kinetics of variants (e.g., RBD variants) to cognate variant receptor (e.g., human ACE2).

For example, in some embodiments, binding kinetics of variants (e.g., RBD variants) was determined using a surface plasmon resonance system (SPRS) (e.g., Biacore T200 device from Cytiva) with an appropriate running buffer (e.g., HBS-EP+ running buffer; BR100669, Cytiva) at 25° C. Carboxyl groups on a SPRS sensor chip (e.g., a CM5 sensor chip matrix from Cytiva) were activated with a mixture of 1-ethyl-3-(3-dimethylaminopropyl) carbodiimidehydrochloride (EDC) and N-hydroxysuccinimide (NHS) to form active esters for the reaction with amine groups. Anti-mouse-Fc-antibody (e.g., BR100838, Cytiva) was diluted in 10 mM sodium acetate buffer pH 5 (30 μg/mL) for covalent coupling to immobilisation level of ˜6,000 response units (RU). Free N-hydroxysuccinimide esters on the sensor surface were deactivated with ethanolamine.

Recombinant proteins of human cognate receptor for variants (e.g., ACE2 with a mFc Tag; ACE2-mFc; 10108-H05H, Sino Biological Inc.) was diluted to 5 μg/mL with HBS-EP+ buffer and applied at 10 μL/min for 15 seconds to the active flow cell for capture by immobilised antibody, while the reference flow cell was treated with buffer. Binding analysis of captured recombinant proteins of human cognate receptor for variants (e.g., hACE2-mFc) to variants (e.g., RBD variants as described in Table 14 below) was performed using a multi-cycle kinetic method with concentrations ranging from about 3 to 50 nM. An association period of 120 seconds was followed by a dissociation period of 300 seconds with a constant flow rate of 30 μL/min and a final regeneration step. Binding kinetics were calculated using a global kinetic fit model (e.g., 1:1 Langmuir, Biacore T200 Evaluation Software Version 3.1, Cytiva).

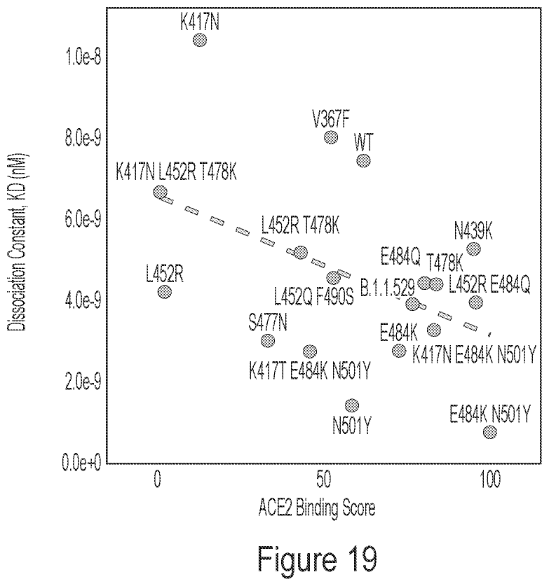

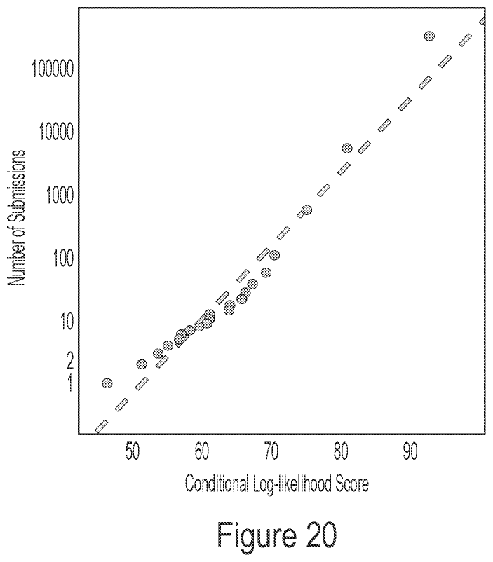

In order to assess the validity of the ACE2 binding score, in some embodiments, SPR kinetic analysis was performed to determine the affinity (K_{D}, dissociation constant) of 19 RBD variants to the ACE2 receptor, and these measured affinities were then compared to the ACE2 binding scores calculated in silico. Notably, the SPR assay measures observable association rates (kₒₙ), which are a result of a dynamic process, while the simulations used to calculate the ACE2 binding score measure aggregated, static binding affinity. In some embodiments, because of the dynamic ACE2 binding process, simulations using static binding affinity may have the potential to marginalize the contribution of mutations that increase the flexibility of the spike protein. Despite the potential marginalization of flexibility-increasing mutations, the ACE2 binding scores showed a meaningful correlation with measured K_{D} values, with a Pearson correlation coefficient of 0.45. (FIG. 19), thus validating use of the ACE2 binding score.

TABLE 14
Exemplary variants assessed for ACE2/RBD binding kinetics

| | | | Cat. No. |
|---|---|---|---|
| Variant | | Mutation | (Sinobiological Inc.) |
| WT | | — | 40592-V08B |
| Lambda | C.37 | L452Q, F490S | 40592-V08H113 |
| Delta/Delta plus | AY.1/AY.2 | K417N, L452R, T478K | 40592-V08H115 |
| Alpha | B.1.1.7 | N501Y | 40592-V08H82 |
| Beta | B.1.351 | K417N | 40592-V08H59 |
| Beta | B.1.351 | E484K | 40592-V08H84 |
| Beta | B.1.351/B.1.351.2/B.1.351.3 | K417N, E484K, N501Y | 40592-V08H85 |
| Iota | B.1.526 | S477N, E484K | 40592-V08H112 |
| | B.1.617 | L452R | 40592-V08H28 |
| | B.1.617 | E484Q | 40592-V08H81 |
| Delta | B.1.617.2 | L452R, T478K | 40592-V08H90 |
| Delta | B.1.617.2 | T478K | 40592-V08H91 |
| Kappa | B.1.617/B.1.617.1/b.1.617.3 | L452R, E484Q | 40592-V08H88 |
| Gamma | P.1/ P.1.1/P.1.2 | K417T, E484K, N501Y | 40592-V08H86 |
| | P.3 | E484K, N501Y | 40592-V08H111 |
| | Mink Variant | Y453F | 40592-V08H80 |
| | — | V367F | 40592-V08H1 |
| | — | N439K | 40592-V08H14 |
| | — | S477N | 40592-V08H46 |
| Omicron | B.1.1.529 | SARS-CoV-2 Spike RBD | 40592-V08H121 |
| | | (YP_009724390.1, with | |
| | | mutation G339D, S371L, | |
| | | S373P, S375F, K417N, | |
| | | N440K, G446S, S477N, | |
| | | T478K, E484A, Q493R, | |
| | | G496S, Q498R, N501Y, | |
| | | Y505H) | |

### Example 5: Comparison of EWS to Standard Machine Learning Methods for Detecting High Risk Variants

A comparison of the detection capabilities of EWS and/or methods (e.g., as described herein) to standard machine learning (ML) methods was performed to highlight both the difficulty of detecting high risk variants and the need for new deep learning approaches. Standard machine learning techniques, both supervised (denoted “GLM with mutations” and corresponding conceptually to Epitope Alteration Score) and unsupervised (denoted “UMAP with mutations” and corresponding conceptually to Semantic Change Score) were used to assess the risk of SARS-CoV-2 variants. As discussed further below, these standard machine learning techniques were not found to reach the same predictive performance as EWS and/or methods described herein.

For each approach, protein sequences were represented by a vector of N binary components. To compute the representation for a protein sequence S deposited at time t, the N most prevalent mutations in all deposited sequences up to time t, inclusive, was calculated. Each binary component of the representation equals 1 if the mutation is present in S and 0 otherwise. N was set to 1280 for each approach, to permit for a direct comparison of the methods.

As the EWS and/or methods disclosed herein learn from unlabeled data, first an unsupervised learning baseline was considered: Uniform Manifold Approximation and Projection (UMAP). This is an intuitive approach, as UMAP has been successfully applied to analogous problems in biology, and it is known to render meaningful insights when applied in life science settings (Becht et al., 2018). UMAP was performed each week over the representations of all sequences available up to the week the experiment was performed. A metric equivalent to the semantic change was computed as a mean L1 distance between the sequence projection and the projections of the Wuhuan and D614G strains in UMAP spaces. The same detection technique as performed by EWS was then used to flag every week a set of 20 variants suspected to be dangerous. Using this approach, 5 out of 13 HRVs were detected, with an average lead time of −43 days (see Table 15). In comparison, EWS was capable of detecting 12 out of 13 variants (8 ahead of time), with a mean lead time of 6 days. These results highlight the need for more involved representations, such as ones learned by Transformers models, in tasks like identifying high risk variants, where significance of novel findings is difficult to approximate a priori.

Next, a supervised learning baseline was considered. Each week, all protein sequences that had been registered were labeled by 1 if they had been named an HRV anytime before or during the week considered and 0 otherwise. A Generalized Linear Model (GLM) was built over the same 1280-dimensional representations of the sequences used in the UMPA approach. The probability of belonging to the HRV class returned by the GLM was then used to rank sequences. Subsequently, the same detection technique performed by EWS was used to flag every week a set of 20 variants suspected to be dangerous. While 12 out of 13 variants were eventually detected using this approach, only 4 out of 13 were detected before WHO designation, with an average lead time of 0.3 days (see Table 15). In addition to performing worse than the methods disclosed herein, the GLM approach is also less generic, meaning that it is implicitly not fully applicable to infectious diseases that attract less worldwide attention than SARS-CoV-2, and have less or no labeled data. In addition, a GLM approach cannot be used early in a pandemic, when there are no labels available and hallmark mutations are unlikely to be among the most common mutations in population.

TABLE 15
Comparison between EWS detection capabilities and three
baselines. Two baselines are based on unsupervised learning
(UMAP) and one baseline is supervised (GLM).

| | | | Number of | |
|---|---|---|---|---|
| | Number of | Number of | Days Ahead | Number |
| | Days Ahead | Days Ahead | (EWS Semantic | Days Ahead |
| HRV | (UMAP) | (GLM) | Change Only) | (EWS) |
| Alpha | −47 | −38 | 65 | 65 |
| Beta | — | −46 | 21 | 21 |
| Gamma | — | 1 | −121 | 1 |
| Delta | −58 | −25 | −78 | — |
| Epsilon | 64 | −27 | 66 | 104 |
| Zeta | — | 75 | — | 103 |
| Eta | — | 90 | 90 | 90 |
| Theta | — | −68 | 13 | 27 |
| Iota | −96 | 79 | −49 | 79 |
| Kappa | — | −25 | −29 | 4 |
| Lambda | −77 | −5 | 77 | 77 |
| Mu | — | −8 | 12 | 122 |
| Omicron | — | — | 3 | 3 |

### REFERENCES CITED

Barnes, C. O., Jette, C. A., Abernathy, M. E., Dam, K.-M. A., Esswein, S. R., Gristick, H. B., Malyutin, A. G., Sharaf, N. G., Huey-Tubman, K. E., Lee, Y. E., et al. (2020). SARS-CoV-2 neutralizing antibody structures inform therapeutic strategies. Nature 588, 682-687.
Berger Rentsch, M., and Zimmer, G. (2011). A vesicular stomatitis virus replicon-based bioassay for the rapid and sensitive determination of multi-species type I interferon. PLoS ONE 6, e25858.
Corey, L., Beyrer, C., Cohen, M. S., Michael, N. L., Bedford, T., and Rolland, M. (2021). SARS-CoV-2 Variants in Patients with Immunosuppression. N. Engl. J. Med. 385, 562-566.
Dejnirattisai, W., Zhou, D., Ginn, H. M., Duyvesteyn, H. M. E., Supasa, P., Case, J. B., Zhao, Y., Walter, T. S., Mentzer, A. J., Liu, C., et al. (2021). The antigenic anatomy of SARS-CoV-2 receptor binding domain. Cell 184, 2183-2200.e22.
Devlin, J., Chang, M.-W., Lee, K., and Toutanova, K. (2018). Bert: Pre-training of deep bidirectional transformers for language understanding. ArXiv Preprint ArXiv:1810.04805.
Elnaggar, A., Heinzinger, M., Dallago, C., Rihawi, G., Wang, Y., Jones, L., Gibbs, T., Feher, T., Angerer, C., Steinegger, M., et al. (2020). ProtTrans: towards cracking the language of Life's code through self-supervised deep learning and high performance computing. ArXiv Preprint ArXiv:2007.06225.
Hatcher, E. L., Zhdanov, S. A., Bao, Y., Blinkova, O., Nawrocki, E. P., Ostapchuck, Y., Schäffer, A. A., and Brister, J. R. (2017). Virus Variation Resource-improved response to emergent viral outbreaks. Nucleic Acids Res. 45, D482-D490.
Hie, B., Zhong, E. D., Berger, B., and Bryson, B. (2021). Learning the language of viral evolution and escape. Science 371, 284-288.
Ju, B., Zhang, Q., Ge, J., Wang, R., Sun, J., Ge, X., Yu, J., Shan, S., Zhou, B., Song, S., et al. (2020). Human neutralizing antibodies elicited by SARS-CoV-2 infection. Nature 584, 115-119.
Kingma, D. P., and Ba, J. (2014). Adam: A method for stochastic optimization. ArXiv Preprint ArXiv:1412.6980.
Liu, J., Liu, Y., Xia, H., Zou, J., Weaver, S. C., Swanson, K. A., Cai, H., Cutler, M., Cooper, D., Muik, A., et al. (2021a). BNT162b2-elicited neutralization of B.1.617 and other SARS-CoV-2 variants. Nature 596, 273-275.
Liu, Y., Liu, J., Xia, H., Zhang, X., Fontes-Garfias, C. R., Swanson, K. A., Cai, H., Sarkar, R., Chen, W., Cutler, M., et al. (2021b). Neutralizing Activity of BNT162b2-Elicited Serum. N. Engl. J. Med. 384, 1466-1468.
Meier, J., Rao, R., Verkuil, R., Liu, J., Sercu, T., and Rives, A. (2021). Language models enable zero-shot prediction of the effects of mutations on protein function. BioRxiv.
Muik, A., Wallisch, A.-K., Sänger, B., Swanson, K. A., Mühl, J., Chen, W., Cai, H., Maurus, D., Sarkar, R., Türeci, Ö., et al. (2021). Neutralization of SARS-CoV-2 lineage B.1.1.7 pseudovirus by BNT162b2 vaccine-elicited human sera. Science 371, 1152-1153.
O'Toole, Á., Scher, E., Underwood, A., Jackson, B., Hill, V., McCrone, J. T., Colquhoun, R., Ruis, C., Abu-Dahab, K., Taylor, B., et al. (2021). Assignment of epidemiological lineages in an emerging pandemic using the pangolin tool. Virus Evol. 7, veab064.
Paszke, A., Gross, S., Massa, F., Lerer, A., Bradbury, J., Chanan, G., Killeen, T., Lin, Z., Gimelshein, N., Antiga, L., et al. (2019). PyTorch: An Imperative Style, High-Performance Deep Learning Library. In Advances in Neural Information Processing Systems 32, H. Wallach, H. Larochelle, A. Beygelzimer, F. dAlché-Buc, E. Fox, and R. Garnett, eds. (Curran Associates, Inc.), pp. 8024-8035.
Rambaut, A., Holmes, E. C., O'Toole, Á., Hill, V., McCrone, J. T., Ruis, C., du Plessis, L., and Pybus, O. G. (2020). A dynamic nomenclature proposal for SARS-CoV-2 lineages to assist genomic epidemiology. Nat. Microbiol. 5, 1403-1407.
Rives, A., Meier, J., Sercu, T., Goyal, S., Lin, Z., Liu, J., Guo, D., Ott, M., Zitnick, C. L., Ma, J., et al. (2021). Biological structure and function emerge from scaling unsupervised learning to 250 million protein sequences. Proc Natl Acad Sci USA 118.
Sahin, U., Muik, A., Vogler, I., Derhovanessian, E., Kranz, L. M., Vormehr, M., Quandt, J., Bidmon, N., Ulges, A., Baum, A., et al. (2021). BNTT62b2 vaccine induces neutralizing antibodies and poly-specific T cells in humans. Nature 595, 572-577.
Shu, Y., and McCauley, J. (2017). GISAID: Global initiative on sharing all influenza data-from vision to reality. Eurosurveillance 22, 30494.
Singh, J., Rahman, S. A., Ehtesham, N. Z., Hira, S., and Hasnain, S. E. (2021). SARS-CoV-2 variants of concern are emerging in India. Nat. Med. 27, 1131-1133.
Steinegger, M., Meier, M., Mirdita, M., Vöhringer, H., Haunsberger, S. J., and Söding, J. (2019). HH-suite3 for fast remote homology detection and deep protein annotation. BMC Bioinformatics 20, 473.
Suzek, B. E., Huang, H., McGarvey, P., Mazumder, R., and Wu, C. H. (2007). UniRef: comprehensive and non-redundant UniProt reference clusters. Bioinformatics 23, 1282-1288.
Tanaka, S., Nelson, G., Olson, C. A., Buzko, O., Higashide, W., Shin, A., Gonzalez, M., Taft, J., Patel, R., Buta, S., et al. (2021). An ACE2 Triple Decoy that neutralizes SARS-CoV-2 shows enhanced affinity for virus variants. Sci. Rep. 11, 12740.
The Technical Advisory Group on SARS-CoV-2 Virus Evolution (TAG-V E) (2021). Classification of Omicron (B.1.1.529): SARS-CoV-2 Variant of Concern.
Twohig, K. A., Nyberg, T., Zaidi, A., Thelwall, S., Sinnathamby, M. A., Aliabadi, S., Seaman, S. R., Harris, R. J., Hope, R., Lopez-Bernal, J., et al. (2021). Hospital admission and emergency care attendance risk for SARS-CoV-2 delta (B.1.617.2) compared with alpha (B.1.1.7) variants of concern: a cohort study. Lancet Infect. Dis.
UniProt Consortium (2019). UniProt: a worldwide hub of protein knowledge. Nucleic Acids Res. 47, D506-D515.
Vaswani, A., Shazeer, N., Parmar, N., Uszkoreit, J., Jones, L., Gomez, A. N., Kaiser, U., and Polosukhin, I. (2017). Attention is all you need. In Advances in Neural Information Processing Systems, pp. 5998-6008.
Weigang, S., Fuchs, J., Zimmer, G., Schnepf, D., Kern, L., Beer, J., Luxenburger, H., Ankerhold, J., Falcone, V., Kemming, J., et al. (2021). Within-host evolution of SARS-CoV-2 in an immunosuppressed COVID-19 patient as a source of immune escape variants. Nat. Commun. 12, 6405.
Weisblum, Y., Schmidt, F., Zhang, F., DaSilva, J., Poston, D., Lorenzi, J. C., Muecksch, F., Rutkowska, M., Hoffmann, H.-H., Michailidis, E., et al. (2020). Escape from neutralizing antibodies by SARS-CoV-2 spike protein variants. ELife 9.
Yan, R., Wang, R., Ju, B., Yu, J., Zhang, Y., Liu, N., Wang, J., Zhang, Q., Chen, P., Zhou, B., et al. (2021). Structural basis for bivalent binding and inhibition of SARS-CoV-2 infection by human potent neutralizing antibodies. Cell Res. 31, 517-525.
Zhang, L., Mann, M., Syed, Z. A., Reynolds, H. M., Tian, E., Samara, N. L., Zeldin, D. C., Tabak, L. A., and Ten Hagen, K. G. (2021). Furin cleavage of the SARS-CoV-2 spike is modulated by O-glycosylation. Proc Natl Acad Sci USA 118.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method for assessing risk for a variant polypeptide, the method comprising:
providing an amino acid sequence of the variant polypeptide, which comprises one or more amino acid modifications relative to one or more reference viral polypeptides;
modeling, utilizing a processing unit of a computing infrastructure, one or more structural features of the variant polypeptide that are involved in viral invasion of a host;
determining, by the processing unit, based on sequence data associated with the viral polypeptide, distance of each of the one or more amino acid modifications relative to corresponding amino acids in the one or more the reference viral polypeptides; and
designating, by the processing unit, the variant polypeptide as a variant with elevated risk when the variant polypeptide is characterized in that:
it has an immune escape score that (i) satisfies a pre-determined immune escape threshold indicating likelihood of the variant polypeptide to be detected and neutralized by antibodies; and/or (ii) is ranked higher than at least one or more other variant polypeptides and/or reference viral polypeptides.

2. A method for assessing risk for a plurality of variant polypeptides, the method comprising:
providing a plurality of amino acid sequences of the variant polypeptides, wherein each of the variant polypeptides comprises one or more amino acid modifications relative to one or more reference viral polypeptides;
ascertaining, utilizing a processing unit of a computing infrastructure, for each of the variant polypeptides, an immune escape score by performing the following processes:
modeling one or more structural features of each variant polypeptide that are involved in viral invasion of a host;
determining, based on sequence data associated with the viral polypeptide, distance of each of the one or more amino acid modifications relative to the corresponding amino acids in the one or more reference viral polypeptides;
ranking, by the processing unit, risk of the variant polypeptides in the plurality by referencing respective immune escape scores; and
designating, by the processing unit, a variant polypeptide as a variant polypeptide with elevated risk when its immune escape score is ranked higher than that of at least one other variant polypeptide in the plurality.

3. The method of claim 2, wherein the variant polypeptide designated as elevated risk is characterized in that:
it has an immune escape score that satisfies a pre-determined immune escape threshold indicating likelihood of the variant polypeptide to be detected and neutralized by antibodies.

4. The method of claim 2, wherein all variant polypeptides of the plurality share an overall amino acid sequence identity of at least 90% with each other.

5. The method of claim 2, wherein all variant polypeptides of the plurality share an overall amino acid sequence identity of at least 90% with the one or more reference viral polypeptides.

6. The method of claim 1, wherein the viral polypeptide is SARS-CoV-2 Spike polypeptide.

7. The method of claim 6, wherein the one or more amino acid modifications are present in Receptor Binding Domain (RBD) or N-terminal domain of the Spike polypeptide.

8. The method of claim 6, wherein calculation of the immune escape score comprises calculation of an epitope alteration score, wherein the epitope alteration score is determined by identifying one or more sequence alterations in the SARS-CoV-2 Spike polypeptide, and comparing the location and/or nature of the one or more sequence alterations to amino acid loci associated with disrupting binding interactions between neutralizing antibodies and a SARS-CoV-2 Spike polypeptide.

9. The method of claim 1, wherein the immune escape score is calculated using a machine learning language model.

10. The method of claim 9, wherein the immune escape score calculation comprises determining a semantic change score for the variant polypeptide relative to the one or more reference viral polypeptides.

11. The method of claim 10, wherein the one or more reference viral polypeptides is or comprises a Wuhan SARS-CoV-2 spike polypeptide or portion thereof.

12. The method of claim 10, wherein the one or more reference viral polypeptides is or comprises a D614G SARS-CoV-2 spike polypeptide or portion thereof.

13. The method of claim 9, wherein the machine learning language model has been trained on a database comprising SARS-CoV-2 polypeptide sequences.

14. (canceled)

15. The method of claim 10, wherein the immune escape score is calculated using a combination of the semantic change score and the epitope alteration score.

16. (canceled)

17. The method of claim 10, wherein at least one of (i) the immune escape score, (ii) the semantic change score, and (iii) the epitope alteration score correlates with a pseudovirus neutralization test result.

18. (canceled)

19. The method of claim 1, wherein the variant polypeptide designated as a variant with elevated risk is characterized in that when assessed with a pseudovirus neutralization assay, the variant polypeptide exhibits a reduction in observed 50% pseudovirus neutralization titer (pVNT₅₀) by at least 30% as compared to the one or more reference viral polypeptides.

20. The method of claim 19, wherein the one or more reference viral polypeptides is or comprises a wild-type SARS-CoV-2 (Wuhan strain) pseudotyped VSV.

21-37. (canceled)

38. A method of producing a vaccine against SARS-CoV-2, the method comprising:
identifying at least one variant polypeptide as a variant polypeptide of interest by:
providing an amino acid sequence of the variant polypeptide, wherein the variant polypeptide comprises one or more amino acid modifications relative to one or more reference viral polypeptides;
determining, utilizing a processing unit of a computing infrastructure, an immune escape score for the variant polypeptide by:
modeling one or more structural features of the variant polypeptide that are involved in viral invasion of a host; and
determining, based on sequence data associated with the viral polypeptide, distance of each of the one or more amino acid modifications relative to corresponding amino acids in the one or more reference viral polypeptides; and
designating, by the processing unit, the variant polypeptide as the at least one variant polypeptide of interest based at least in part on the immune escape score; and
producing, within no more than 1 month from the identification of the at least one variant polypeptide of interest, a vaccine comprising a polypeptide or a nucleic acid encoding the polypeptide, wherein the polypeptide comprises the at least one variant polypeptide of interest or immunogenic fragment thereof.

39-41. (canceled)

42. A SARS-CoV-2 Spike polypeptide or an immunogenic fragment or variant thereof, or a nucleic acid comprising a sequence encoding the same, wherein the Spike polypeptide or the immunogenic fragment or variant is determined as a variant of concern by:
providing an amino acid sequence of the Spike polypeptide or the immunogenic fragment or variant, said Spike polypeptide or immunogenic fragment or variant thereof comprising one or more amino acid modifications relative to one or more reference viral polypeptides;
determining, utilizing a processing unit of a computing infrastructure, an immune escape score for the Spike polypeptide or the immunogenic fragment or variant by:
modeling one or more structural features of the Spike polypeptide or the immunogenic fragment or variant that are involved in viral invasion of a host; and
determining, based on sequence data associated with the Spike polypeptide or the immunogenic fragment or variant, a distance of each of the one or more amino acid modifications relative to corresponding amino acids in the one or more reference viral polypeptides; and
designating, by the processing unit, the Spike polypeptide or the immunogenic fragment or variant as a variant of concern based at least in part on the immune escape score.

43-59. (canceled)

60. The method of claim 1, wherein the immune escape score is based (i) on the determined distance of each of the one or more amino acid modifications and (ii) calculation of an epitope alteration score.

61. The method of claim 60, wherein the epitope alteration score is determined by identifying one or more sequence alterations in the variant polypeptide, and comparing the location and/or nature of the one or more sequence alterations to amino acid loci associated with disrupting binding interactions between neutralizing antibodies and the one or more reference viral polypeptides.

62. The method of claim 1, comprising designating, by the processing unit, the variant polypeptide as a variant with elevated risk when the variant polypeptide is characterized further in that it has an infectivity score that (i) satisfies a pre-determined infectivity threshold indicating level of viral fitness; and/or (ii) is ranked higher than at least one or more other variant polypeptides and/or reference viral polypeptides.

63. The method of claim 62, comprising calculating of the infectivity score based on calculation of an ACE2 binding score, wherein the ACE2 binding score is a measure of binding affinity between an ACE2 receptor and a Spike polypeptide.

64. The method of claim 2, wherein the modeling of the one or more structural features of each variant polypeptide comprises calculating an epitope alteration score, wherein the epitope alteration score is determined by identifying one or more sequence alterations in each variant polypeptide, and comparing the location and/or nature of the one or more sequence alterations to amino acid loci associated with disrupting binding interactions between neutralizing antibodies and the one or more reference viral polypeptides.

65. The method of claim 2, comprising:
ascertaining, by the processing unit, for each of the variant polypeptides, an infectivity score; and
ranking, by the processing unit, risk of the variant polypeptides in the plurality by referencing respective combined scores of the immune escape score and the infectivity score.
